(19) United States
(12) Patent Application Publication
(10) Pub. No.: US 2012/0107264 A1
(43) Pub. Date: May 3, 2012

(54) NUCLEIC ACID DELIVERY COMPOUNDS

(75) Inventors: Padmanabh Chivukula, San Diego, CA (US); Jian Liu, San Diego, CA (US); Akinobu Soma, Carlsbad, CA (US); Keisaku Okada, San Diego, CA (US); Sang Van, San Diego, CA (US); Julia Zhu, Escondido, CA (US); Nianchun Ma, Oceanside, CA (US); Chen-Chang Lee, Oceanside, CA (US); Fu Chen, Carlsbad, CA (US); Lei Yu, Carlsbad, CA (US)

(73) Assignee: Nitto Denko Corporation, Osaka (JP)

(21) Appl. No.: 13/273,106

(22) Filed: Oct. 13, 2011

Related U.S. Application Data
(60) Provisional application No. 61/393,297, filed on Oct. 14, 2010.
(60) Provisional application No. 61/427,981, filed on Dec. 29, 2010.
(60) Provisional application No. 61/454,922, filed on Mar. 21, 2011.

(51) Int. Cl. A61K 47/48; C08F 220/60; A61P 35/00; A61K 47/32; C12N 5/071; C08F 120/60; C08G 65/333

(52) U.S. Cl. 424/78.27; 525/328.4; 525/54.2; 525/421; 514/772.1; 435/375

(57) ABSTRACT

Polymers including two or more different recurring units are disclosed herein. Also disclosed herein are methods of using such polymers to deliver nucleic acids to a cell.

### RELATED APPLICATION INFORMATION

This application claims priority to U.S. Provisional Application Ser. No. 61/393,297, filed on Oct. 14, 2010; U.S. Provisional Application Ser. No. 61/427,981, filed on Dec. 29, 2010; and U.S. Provisional Application Ser. No. 61/454,922, filed on Mar. 21, 2011; which are incorporated herein by reference in their entireties for all purposes.

### SEQUENCE LISTING

The present application is being filed along with a Sequence Listing in electronic format. The Sequence Listing is provided as a file entitled KUZU1_{—}017P2_SequenceListing.TXT, created Oct. 7, 2011, which is 6.51 kb in size. The information in the electronic format of the Sequence Listing is incorporated herein by reference in its entirety.

### BACKGROUND

1. Field

Disclosed herein are compositions and methods related to the fields of organic chemistry, pharmaceutical chemistry, biochemistry, molecular biology, and medicine. More specifically, embodiments described herein relate to compounds, compositions, and methods for delivering a nucleic acid into a cell.

2. Description

A number of techniques are available for delivering a nucleic acid such as siRNA into a cell, including the use of viral transfection systems and non-viral transfection systems. Non-viral transfection systems can include, for example, polymers, lipids, liposomes, micelles, dendrimers, and nanomaterials. Examples of polymers that have previously been studied for cell transfection include some cationic polymers.

Each type of system has its respective advantages and drawbacks. For example, viral systems can yield high transfection efficiency, but may not be as safe as some non-viral systems. (See Verma I M et al. Nature (1997) 389: 239-242; Marshall E. Science (2000) 286: 2244-2245). In addition, viral systems can be complicated and/or expensive to prepare. Non-viral transfection systems, such as cationic polymers, have been reported to transfer plasmid DNA into cells. Some drawbacks to the use of some cationic polymers include their toxicity to cells and/or their lack of stability.

### SUMMARY

Some embodiments disclosed herein relate to a polymer that can include a first recurring unit of Formula (I) and a second recurring unit of Formula (II).

Some embodiments disclosed herein relate to a polymer that can include a first recurring unit of Formula (I), a second recurring unit of Formula (II), and a third recurring unit of Formula (III).

Some embodiments disclosed herein relate to a polymer that can include a recurring unit of Formula (I), a recurring unit of Formula (II), and a recurring unit of Formula (XIII).

Some embodiments disclosed herein relate to a polymer that can include a recurring unit of Formula (I), a recurring unit of Formula (II), a recurring unit of Formula (III), and a recurring unit of Formula (XIII).

Some embodiments disclosed herein relate to a block copolymer that can include a first subunit that can include a recurring unit of Formula (E) and/or a recurring unit of Formula (F) and a second subunit that can include a polymer disclosed herein.

In some embodiments, a polymer described herein can be associated with a nucleic acid. For example, the nucleic acid can be selected from DNA, RNA siRNA and antisense.

Some embodiments described herein related to a pharmaceutical composition that can include a polymer described herein and a pharmaceutically acceptable carrier. In some embodiments, the pharmaceutical composition can further include a nucleic acid.

Some embodiments described herein relate to a method of transfecting a cell that can include delivering to a cell a polymer described herein associated with a nucleic acid. Other embodiments described herein relate to using a polymer described herein associated with a nucleic acid in the preparation of a medicament for transfecting a cell. Still other embodiments described herein relate to a polymer described herein associated with a nucleic acid for transfecting a cell.

Some embodiments described herein relate to a method of treating a tumor that can include administering an effective amount of a polymer described herein associated with a nucleic acid. Other embodiments described herein relate to using a polymer described herein associated with a nucleic acid in the preparation of a medicament for treating a tumor. Still other embodiments described herein relate to a polymer described herein associated with a nucleic acid for treating a tumor. Some embodiments described herein relate to a method of treating a tumor that can include contacting a tumor cell with an effective amount of a polymer described herein associated with a nucleic acid.

### BRIEF DESCRIPTION OF THE DRAWINGS

FIG. 1 is a schematic illustrating an example of a polymer with a group having a pH transition point.

FIGS. 2-17, 19, and 21 each depict a reaction scheme for the synthesis of a polymer that includes a first recurring unit, a second recurring unit, a third recurring unit, and a fourth recurring unit that includes a group that has a pH transition point.

FIG. 18 depicts a polymer that includes a first recurring unit, a second recurring unit, and a third recurring unit that includes a group that has a pH transition point.

FIG. 20 depicts a polymer that includes a first recurring unit, a second recurring unit, a third recurring unit, and a fourth recurring unit that includes a group that has a pH transition point.

FIG. 22 illustrates two bar graphs each depicting the results of a siRNA transfection assay for several polymers described herein.

FIGS. 23-28 each depict a reaction scheme for the synthesis of a polymer that includes a first recurring unit, a second recurring unit, a third recurring unit, and a fourth recurring unit that includes a group that has a pH transition point.

FIG. 29 illustrates a bar graph depicting the results of a hemolysis assay for several polymers described herein.

FIGS. 30 and 31 each depict a polymer that includes a first recurring unit, a second recurring unit, a third recurring unit, and a fourth recurring unit that includes a group that has a pH transition point.

FIGS. 32-35 each illustrate a bar graph depicting the results of a siRNA transfection assay for several polymers described herein.

FIG. 36 depicts DNA (gene) transfection efficiency for several polymers.

FIG. 37 illustrates a bar graph depicting the results of a hemolysis assay for several polymers described herein.

FIG. 38 illustrates a bar graph depicting the results of a hemolysis assay for several polymers described herein.

FIGS. 39A-B each depict a reaction scheme for the synthesis of a polymer that includes a first recurring unit, a second recurring unit, a third recurring unit, and a fourth recurring unit that includes a group that has a pH transition point, and that is covalently bonded to a nucleic acid.

FIGS. 40-41 each depict a reaction scheme for the synthesis of a polymer that includes a first recurring unit, a second recurring unit, a third recurring unit, and a fourth recurring unit that includes a group that has a pH transition point.

FIGS. 42-44 each depict ¹H NMR spectra for a polymer described herein that includes a first recurring unit, a second recurring unit, a third recurring unit, and a fourth recurring unit that includes a group that has a pH transition point.

FIG. 45 illustrates the results of a gel electrophoresis assay conducted on a polymer described herein that includes a first recurring unit, a second recurring unit, a third recurring unit, and a fourth recurring unit that includes a group that has a pH transition point, and that is covalently bonded to a nucleic acid.

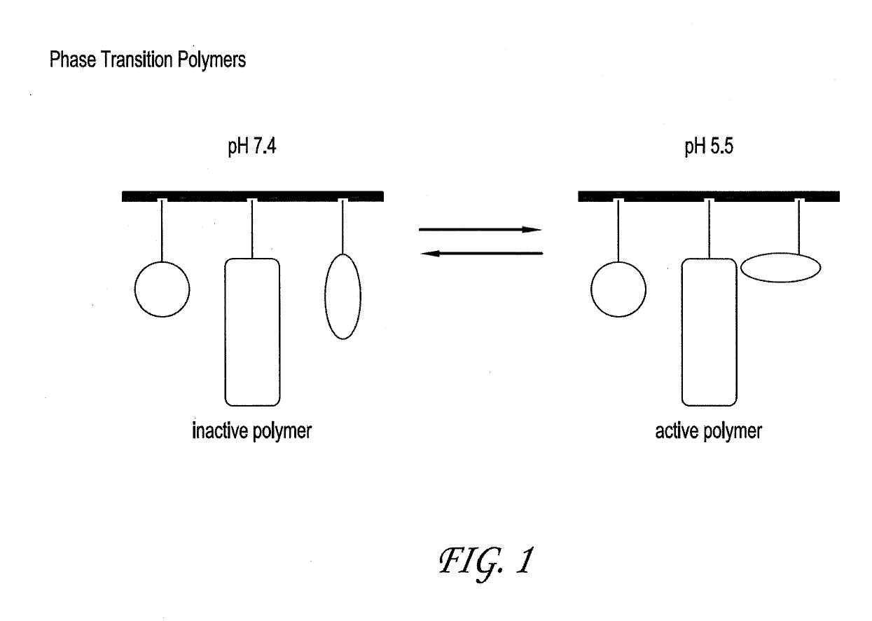

### DETAILED DESCRIPTION

Unless defined otherwise, all technical and scientific terms used herein have the same meaning as is commonly understood by one of ordinary skill in the art. All patents, applications, published applications and other publications referenced herein are incorporated by reference in their entirety. In the event that there are a plurality of definitions for a term herein, those in this section prevail unless stated otherwise.

As used herein, the term “pH transition point” refers to the pH above or below which a group having a pH transition point experiences a change in one or more chemical properties. Examples of such chemical properties include, but are not limited to, hydrophilicity, lipophilicity, solubility, polarity, and electric charge.

Where at least two molecules are “associated” it means that the molecules are in electronic interaction with each other. Such interaction may take the form of a chemical bond, including, but not limited to, a covalent bond, a polar covalent bond, an ionic bond, an electrostatic bond, a coordinate covalent bond, an aromatic bond, a hydrogen bond, a dipole, or a van der Waals interaction. Those of ordinary skill in the art understand that the relative strengths of such interactions may vary widely.

As used herein, “Cₘ to Cₙ” in which “m” and “n” are integers refers to the number of carbon atoms in an alkyl or alkenyl group. That is, the alkyl or alkenyl can contain from “m” to “n”, inclusive, carbon atoms. Thus, for example, a “C₁ to C₄ alkyl” group refers to all alkyl groups having from 1 to 4 carbons, that is, CH₃—, CH₃CH₂—, CH₃CH₂CH₂—, (CH₃)₂CH—, CH₃CH₂CH₂CH₂—, CH₃CH₂CH(CH₃)— and (CH₃)₃C—. If no “m” and “n” are designated with regard to an alkyl or alkenyl group, the broadest range described in these definitions is to be assumed.

As used herein, “alkyl” refers to a straight or branched hydrocarbon chain fully saturated (no double or triple bonds) hydrocarbon group.

As used herein, “alkenyl” refers to an alkyl group that contains in the straight or branched hydrocarbon chain one or more double bonds.

Whenever a group is described as being “optionally substituted” that group may be unsubstituted or substituted with one or more of the indicated substituents. Likewise, when a group is described as being “unsubstituted or substituted” if substituted, the substituent may be selected from one or more the indicated substituents.

Unless otherwise indicated, when a substituent is deemed to be “optionally substituted,” or “substituted” it is meant that the substituent is a group that may be substituted with one or more group(s) individually and independently selected from C₁-C₁₂ alkyl, C₂-C₁₂ alkenyl, C₂-C₁₂ alkynyl, C₃-C₁₂ cycloalkyl, C₃-C₁₂ cycloalkenyl, C₃-C₁₂ cycloalkynyl, C₅-C₁₂ aryl, heteroaryl (containing 5-12 atoms, wherein 1-3 atoms are heteroatoms selected from O, N, and S, and the remaining atoms are carbon), heteroalicyclyl (containing 5-12 atoms, wherein 1-3 atoms are heteroatoms selected from O, N, and S, and the remaining atoms are carbon), C₄-C₂₄ aralkyl, heteroaralkyl (containing 6-24 atoms, wherein 1-3 atoms are heteroatoms selected from O, N, and S, and the remaining atoms are carbon), (heteroalicyclyl)alkyl (containing 6-24 atoms, wherein 1-3 atoms are heteroatoms selected from O, N, and S, and the remaining atoms are carbon), hydroxy, alkoxy, aryloxy, acyl (—C═OR, wherein R is alkyl, cycloalkyl, or aryl), ester, mercapto, cyano, halogen, carbonyl, thiocarbonyl, O-carbamyl, N-carbamyl, O-thiocarbamyl, N-thiocarbamyl, C-amido, N-amido, S-sulfonamido, N-sulfonamido, C-carboxy, O-carboxy, isocyanato, thiocyanato, isothiocyanato, nitro, silyl, sulfenyl, sulfinyl, sulfonyl, haloalkyl (mono-, di- and tri-substituted haloalkyl), haloalkoxy (mono-, di- and tri-substituted haloalkoxy), and amino, including mono- and di-substituted amino groups.

It is understood that, in any compound described herein having one or more chiral centers, if an absolute stereochemistry is not expressly indicated, then each center may independently be of R-configuration or S-configuration or a mixture thereof. Thus, the compounds provided herein may be enantiomerically pure or be stereoisomeric mixtures. In addition it is understood that, in any compound having one or more double bond(s) generating geometrical isomers that can be defined as E or Z each double bond may independently be E or Z or a mixture thereof. Likewise, all tautomeric forms are also intended to be included.

As used herein, the abbreviations for any protective groups, amino acids and other compounds are, unless indicated otherwise, in accord with their common usage, recognized abbreviations, or the IUPAC-IUP Commission on Biochemical Nomenclature (See, Biochem. 11:942-944 (1972)).

Some embodiments herein are directed to a polymer that can include a first recurring unit of Formula (I) and a second recurring unit of Formula (II). The recurring units of Formulae (I) and (II) can each have the following structures:

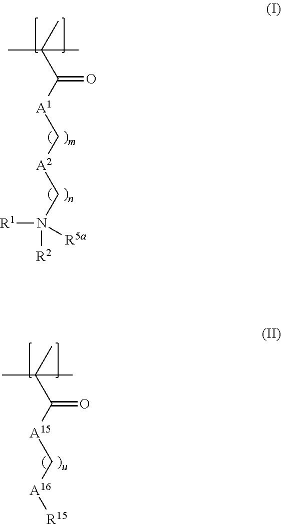

In Formulae (I) and (II), A¹ and A¹⁵ can be independently selected from an oxygen atom and —N(R^{N}), wherein R^{N} is H or C₁-C₆ alkyl; A² can be a bond or —S—S—; A¹⁶ can be a bond, NH, or NHC(═O); m can be 1, 2, 3, 4, 5, or 6; n and u can each independently be 0, 1, 2, 3, 4, 5, or 6; R¹ and R² can each independently selected from H and C₁-C₆ alkyl; Rya can be absent or H, and if R^{5a} is H, the nitrogen atom to which R^{5a} is attached can have an associated positive charge; and R¹⁵ can be selected from C₄-C₂₄ alkyl, C₄-C₂₄ alkenyl, and an optionally substituted steryl.

In some embodiments, R^{5a} can be absent. In some embodiments, R^{5a} can be H, and the nitrogen atom to which R^{5a} is attached can have an associated positive charge. In some embodiments, R¹ and/or R² can each be H. In other embodiments, R¹ and/or R² can each be C₁-C₂ alkyl. In some embodiments, A¹ can be NH or an oxygen atom. In some embodiments, A² can be a bond. In other embodiments, A² can be —S—S—. In some embodiments, m can be 2 or 3. In some embodiments, n can be 0, 1, or 2. In some embodiments, u can be 0, 1, 2, 3, or 4. In some embodiments, A¹ can be NH, A² can be a bond, R¹ and R² can each be H, m can be 3, and n can be 0. In other embodiments, A¹ can be an oxygen atom, A² can be a bond, R¹ and R² can each be H, m can be 2, and n can be 0. In yet other embodiments, A¹ can be NH, A² can be —S—S—, R¹ and R² can each be H, m can be 2, and n can be 2. In still other embodiments, A¹ can be NH, A² can be a bond, R¹ and R² can each be H, m can be 2, and n can be 0. In still yet other embodiments, A¹ can be an oxygen atom, A² can be a bond, R¹ and R² can each be —CH₃, m can be 2, and n can be 0.

In some embodiments, the first recurring unit can have a structure represented by Formula (Ia):

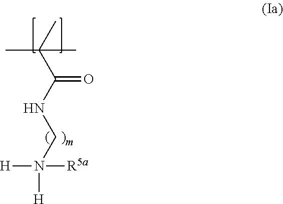

In some embodiments, A¹⁵ can be NH or an oxygen atom. In some embodiments, R¹⁵ can be a substituent selected from oleyl, lauryl, myristyl, palmityl, margaryl, stearyl, arachidyl, behenyl, lignoceryl and optionally substituted steryl. In other embodiments, R¹⁵ can be an optionally substituted C₆-C₁₈ alkyl, an optionally substituted C₄-C₈ alkyl, an optionally substituted C₄-C₁₀ alkyl, an optionally substituted C₄-C₁₅ alkyl, an optionally substituted C₁₅-C₂₀ alkyl, an optionally substituted C₄-C₂₀ alkyl, or an optionally substituted C₁₅-C₂₄ alkyl. In yet other embodiments, R¹⁵ can be an optionally substituted C₆-C₁₈ alkenyl, an optionally substituted C₄-C₈ alkenyl, an optionally substituted C₄-C₁₀ alkenyl, an optionally substituted C₄-C₁₅ alkenyl, an optionally substituted C₁₅-C₂₀ alkenyl, an optionally substituted C₄-C₂₀ alkenyl, or an optionally substituted C₁₅-C₂₄ alkenyl. In some embodiments, R¹⁵ can be a C₁₋₇ alkenyl. For example, R¹⁵ can be —(CH₂)₇CH═CH(CH₂)₇CH₃. In another embodiment, R¹⁵ can be a C₆-C₈ alkyl. In yet other embodiments, R¹⁵ can be an optionally substituted cholesterol. Advantageously, in some embodiments, R¹⁵ can be a lipophilic group. In some embodiments, A¹⁵ can be an oxygen atom, A¹⁶ can be a bond, R¹⁵ can be C₄-C₈ alkyl, and u can be 0. In other embodiments, A¹⁵ can be NH, A¹⁶ can be NHC(═O), R¹⁵ can be C₄-C₂₄ alkyl, and u can be 3.

In some embodiments, the second recurring unit can have a structure selected from Formula (IIa) and (IIb):

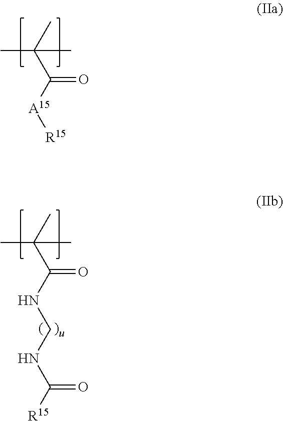

In some embodiments, the polymer can include a third recurring unit having a structure represented by Formula (III):

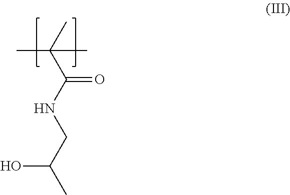

In some embodiments, the polymer can also include a fourth recurring unit that having a structure represented by Formula (A), wherein a is an integer in the range of from about 1 to about 500 and R²¹ can be C₁ to C₆ alkyl:

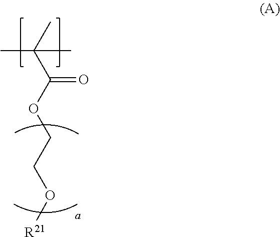

In some embodiments, R²¹ can be methyl or ethyl. In some embodiments, a can be an integer in the range of from about 1 to about 100. In some embodiments the recurring unit of Formula (A) can have a weight average molecular weight in the range of from about 125 Da to about 6000 Da. In other embodiments, the recurring unit of Formula (A) can have a weight average molecular weight in the range of from about 125 Da to about 600 Da. In other embodiments, the recurring unit of Formula (A) can have a weight average molecular weight in the range of from about 125 Da to about 300 Da.

The recurring unit of Formula (A) can be coupled to a recurring unit of Formula (I), (II), and/or (III). In some embodiments, the recurring unit of Formula (A) can be directly coupled to the polymer. In other embodiments, the recurring unit of Formula (A) can be coupled to the polymer through a linking group. Examples of linking groups include, but are not limited to, low molecular weight linking groups comprising 1-12 atoms, such as acrylate, alkylene, amide, ester, carbonate, carbonyl, ether, thioamide and combinations thereof, and high molecular weight linking groups such as polyethylene glycol (PEG).

In some embodiments, the polymer can also include a fifth recurring unit of Formula (XIII):

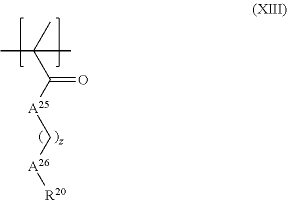

The recurring unit of Formula (XIII) can be coupled to a recurring unit of Formulae (I), (II), (III), and/or (A). In Formula (XIII), A²⁵ can be selected from an oxygen atom and —N(R^{Q}), wherein R^{Q} is H or C₁-C₆ alkyl; A²⁶ can each independently be a bond, NH, or NHC(═O); z can be 0, 1, 2, 3, 4, 5, or 6; and R²⁰ can include a group that has a pH transition point.

In some embodiments, the recurring unit of Formula (XIII) can have a structure selected from Formula (XIIIa) and (XIIIb):

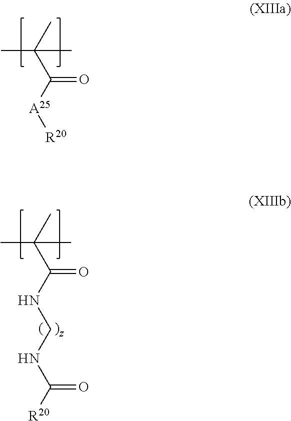

As described herein, R²⁰ can include a group that has a pH transition point. Above or below the pH transition point, the R²⁰ group can experience a change in one or more chemical properties. In some embodiments, R²⁰ can be hydrophilic at a pH that is greater than or equal to the pH transition point. In other embodiments, R²⁰ can be hydrophobic at a pH that is less than the pH transition point. In some embodiments, R²⁰ can be hydrophilic at a pH that is greater than or equal to the pH transition point and hydrophobic at a pH that is less than the pH transition point.

The position of the pH transition point can be controlled by selecting the appropriate chemical structure for R²⁰. In some embodiments, the transition point can be at a pH<7.4 (e.g., less than physiological pH and/or less than the pH of blood). In other embodiments, the transition point can be at a pH<6. In yet other embodiments, the transition point can be at a pH<5. In one embodiment, the transition point can be at a pH that is generally equal to the pH of the micro-environment of tumor tissue (e.g., in or substantially adjacent to tumor tissue). Those skilled in the art may appreciate that the micro-environment of tumor tissue can be acidic relative to physiological pH. Advantageously, in some embodiments R²⁰ can be hydrophilic in a generally physiological environment and can be generally hydrophobic at or substantially adjacent to tumor tissue.

A wide variety of groups having a pH transition point can be used. In some embodiments, a group having a pH transition point can be directly bonded to the A²⁵ group of the recurring unit of Formula (XIII). In other embodiments, a group having a pH transition point can be bonded to the A²⁵ group of the recurring unit of Formula (XIII) through a linking group. Examples of linking groups include, but are not limited to, low molecular weight linking groups comprising 1-12 atoms, such as acrylate, alkylene, amide, amine, ester, carbonate, carbonyl, ether, thioamide and combinations thereof, and high molecular weight linking groups such as polyethylene glycol (PEG). Examples of groups having a pH transition point include, but are not limited to, amines (cyclic and aliphatic; amino, mono-substituted amines, and di-substituted amines), morpholine and carboxylates. In some embodiments, a group having a pH transition point, for example, R²⁰, can be a group that comprises an imidazolyl group, a

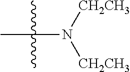

group, a

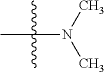

group or a morpholinyl group. In some embodiments, R²⁰ can have the following structure: —C(═O)—(CH₂)₁₋₄—C(═O)OR^{B} at a pH≧the transition point, wherein R^{B} can be an alkali metal, such as sodium. In some embodiments, R²⁰ can have the following structure: —C(═O)—(CH₂)₂—C(═O)OR^{B} at a pH≧the transition point, wherein R^{B} can be an alkali metal, such as sodium. In other embodiments, R²⁰ can have the following structure:

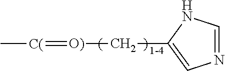

at a pH≧the transition point. In still other embodiments, R²⁰ can have the following structure:

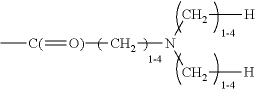

at a pH≧the transition point. In yet still other embodiments, R²⁰ can have the following structure:

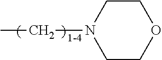

at a pH≧the transition point. In some embodiments, R²⁰ can have the following structure:

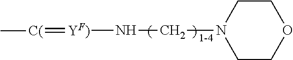

at a pH≧the transition point, wherein Y^{F} is S or O.

The polymer can also include a nucleic acid. The nucleic acid can be associated with the polymer in a variety of ways. In some embodiments, the nucleic acid can be associated with the polymer via an electrostatic bond. In some embodiments, the nucleic acid can be selected from DNA, RNA, siRNA, and antisense. In some embodiments, the nucleic acid can be siRNA. In some embodiments, the nucleic acid, such as siRNA, can be associated with at least one recurring unit of Formula (I). For example, the nucleic acid can be associated with the NR¹R²R^{5a} group of a recurring unit of Formula (I). In some embodiments, the nucleic acid can have a biological effect upon a cell to which it is delivered, in vitro, ex vivo and/or in vivo. For example, those skilled in the art will appreciate that reference herein to the use of a polymer for therapeutic and/or treatment purposes may refer to the use of the polymer in combination with a nucleic acid with which it is associated.

In some embodiments, the nucleic acid can be associated with the polymer via a covalent bond. In some embodiments where the nucleic acid and polymer are associated via a covalent bond, the nucleic acid can be directly covalently bonded to the polymer. In other embodiments, the nucleic acid can be indirectly bonded to the polymer through a linking group. Examples of linking groups include, but are not limited to, low molecular weight linking groups comprising 1-12 atoms, such as acrylate, alkylene, amide, amine, ester, carbonate, carbonyl, ether, thioamide and combinations thereof, and high molecular weight linking groups such as polyethylene glycol (PEG). In some embodiments, the polymer can be covalently bonded to a thiol-reactive agent, including but not limited to iodoacetamide, maleimide, benzylic halide, bromomethylketone, and orthopyridyldisulfide reagent. In some embodiment, the thiol-reactive agent can be covalently bonded to the terminal NR¹R²R^{5a} group of at least one recurring unit of Formula (I) through a PEG linking group. In one embodiment, the linking group can be —(CH₂CH₂O)ₙ—, wherein n is an integer in the range of from about 1 to about 25. In some embodiments, n can be an integer in the range of from about 1 to 5. In other embodiments, n can be an integer in the range of from about 12 to about 24. In one embodiment, n can be 4. In some embodiments, a nucleic acid can be modified to include a thiol group according to methods known to those skilled in the art. In some embodiments, the thiol group on the nucleic acid can be covalently bonded directly to the thiol-reactive group on the polymer. In some embodiments, the nucleic acid can be selected from DNA, RNA, siRNA, and antisense. In some embodiments, the nucleic acid can be siRNA. In some embodiments, the nucleic acid has a biological effect upon a cell to which it is delivered, in vitro, ex vivo and/or in vivo. For example, those skilled in the art will appreciate that reference herein to the use of a polymer for therapeutic or treatment purposes may refer to the use of the polymer in combination with the nucleic acid with which it is associated.

Some embodiments herein are directed to a polymer that can include a first recurring unit of Formula (I) and a second recurring unit of Formula (II). Other embodiments are directed to a polymer that can include a first recurring unit of Formula (I), a second recurring unit of Formula (II), and one or more additional recurring units of Formulae (III), (A), and/or (XIII). In some embodiments, the polymer can include a recurring unit of Formula (I) and a recurring unit of Formula (II). In other embodiments, the polymer can include a recurring unit of Formula (I), a recurring unit of Formula (II), and a recurring unit of Formula (III). In other embodiments, the polymer can include a recurring unit of Formula (I), a recurring unit of Formula (II), and a recurring unit of Formula (A). In other embodiments, the polymer can include a recurring unit of Formula (I), a recurring unit of Formula (II), a recurring unit of Formula (III), and a recurring unit of Formula (A). In other embodiments, the polymer can include a recurring unit of Formula (I), a recurring unit of Formula (II), and a recurring unit of Formula (XIII). In other embodiments, the polymer can include a recurring unit of Formula (I), a recurring unit of Formula (II), a recurring unit of Formula (III), and a recurring unit of Formula (XIII). In other embodiments, the polymer can include a recurring unit of Formula (I), a recurring unit of Formula (II), a recurring unit of Formula (A), and a recurring unit of Formula (XIII). In other embodiments, the polymer can include a recurring unit of Formula (I), a recurring unit of Formula (II), a recurring unit of Formula (III), a recurring unit of Formula (A), and a recurring unit of Formula (XIII). In other embodiments, the polymer can include a recurring unit of Formula (II), a recurring unit of Formula (III), and a recurring unit of Formula (XIII) (e.g., the polymer may not include a recurring unit of Formula (I)). In other embodiments, the polymer can include a recurring unit of Formula (II), a recurring unit of Formula (III), and a recurring unit of Formula (A) (e.g., the polymer may not include a recurring unit of Formula (I)). In other embodiments, the polymer can include a recurring unit of Formula (II), a recurring unit of Formula (III), a recurring unit of Formula (XIII), and a recurring unit of Formula (A) (e.g., the polymer may not include a recurring unit of Formula (I)).

The relative amounts of the first recurring unit of Formula (I), second recurring unit of Formula (II), and any other additional recurring units (e.g., third recurring unit of Formula (III), fourth recurring unit of Formula (A), and/or fifth recurring unit of Formula (XIII)) present in the polymer can vary widely. In some embodiments, the polymer can include ≧90% mole percent of the recurring unit of Formula (I) based on the ratio of total moles of recurring units of Formula (I) to the total moles of recurring units in the polymer. In some embodiments, the polymer can include ≧80% mole percent of the recurring unit of Formula (I) based on the ratio of total moles of recurring units of Formula (I) to the total moles of recurring units in the polymer. In some embodiments, the polymer can include ≧50% mole percent of the recurring unit of Formula (I) based on the ratio of total moles of recurring units of Formula (I) to the total moles of recurring units in the polymer. In some embodiments, the polymer can include ≧30% mole percent of the recurring unit of Formula (I) based on the ratio of total moles of recurring units of Formula (I) to the total moles of recurring units in the polymer. In some embodiments, the polymer can include ≧15% mole percent of the recurring unit of Formula (I) based on the ratio of total moles of recurring units of Formula (I) to the total moles of recurring units in the polymer. In some embodiments, the polymer can include ≧5% mole percent of the recurring unit of Formula (I) based on the ratio of total moles of recurring units of Formula (I) to the total moles of recurring units in the polymer.

In some embodiments, the polymer can include a total amount of the recurring unit of Formula (I) in the range of about 10 mole % to about 70 mole % based on the ratio of total moles of recurring units of Formula (I) to the total moles of recurring units in the polymer. In some embodiments, the polymer can include a total amount of the recurring unit of Formula (I) in the range of about 20 mole % to about 60 mole % based on the ratio of total moles of recurring units of Formula (I) to the total moles of recurring units in the polymer. In some embodiments, the polymer can include a total amount of the recurring unit of Formula (I) in the range of about 30 mole % to about 50 mole % based on the ratio of total moles of recurring units of Formula (I) to the total moles of recurring units in the polymer. In some embodiments, the polymer can include a total amount of the recurring unit of Formula (I) in the range of from about 5 mole % to about 20 mole % based on the ratio of total moles of recurring units of Formula (I) to the total moles of recurring units in the polymer. In some embodiments, the polymer can include a total amount of the recurring unit of Formula (I) of about 10 mole %, about 20 mole %, about 30 mole %, about 40 mole %, about 50 mole %, about 60 mole % or about 70 mole % based on the ratio of total moles of recurring units of Formula (I) to the total moles of recurring units in the polymer.

In some embodiments, the polymer can include at least about 50 recurring units of Formula (I). In other embodiments, the polymer can include at least about 100 recurring units of Formula (I). In other embodiments, the polymer can include at least about 200 recurring units of Formula (I). In other embodiments, the polymer can include at least about 500 recurring units of Formula (I). In other embodiments, the polymer can include at least about 1000 recurring units of Formula (I). In other embodiments, the polymer can include at least about 1500 recurring units of Formula (I). In other embodiments, the polymer can include about 2000 recurring units of Formula (I).

In some embodiments, the polymer can include from about 50 to about 2000 recurring units of Formula (I). In other embodiments, the polymer can include 200 to about 1500 recurring units of Formula (I). In yet other embodiments, the polymer can include about 300 to about 700 recurring units of Formula (I). In other embodiments, the polymer can include from about 50 to about 100 recurring units, from about 1500 to about 2000 recurring units, from about 1000 to about 1500 recurring units, or from about 700 to about 1000 recurring units of Formula (I). In yet other embodiments, the polymer can include from about 100 to about 200, from about 100 to about 500, or from about 300 to about 600 recurring units of Formula (I).

In some embodiments, the polymer can include ≧90% mole percent of the recurring unit of Formula (II) based on the ratio of total moles of recurring units of Formula (II) to the total moles of recurring units in the polymer. In some embodiments, the polymer can include ≧80% mole percent of the recurring unit of Formula (II) based on the ratio of total moles of recurring units of Formula (II) to the total moles of recurring units in the polymer. In some embodiments, the polymer can include ≧50% mole percent of the recurring unit of Formula (II) based on the ratio of total moles of recurring units of Formula (II) to the total moles of recurring units in the polymer. In some embodiments, the polymer can include ≧30% mole percent of the recurring unit of Formula (II) based on the ratio of total moles of recurring units of Formula (II) to the total moles of recurring units in the polymer. In some embodiments, the polymer can include ≧15% mole percent of the recurring unit of Formula (II) based on the ratio of total moles of recurring units of Formula (II) to the total moles of recurring units in the polymer.

In some embodiments, the polymer can include a total amount of the recurring unit of Formula (II) in the range of about 10 mole % to about 70 mole % based on the ratio of total moles of recurring units of Formula (II) to the total moles of recurring units in the polymer. In some embodiments, the polymer can include a total amount of the recurring unit of Formula (II) in the range of about 20 mole % to about 60 mole % based on the ratio of total moles of recurring units of Formula (II) to the total moles of recurring units in the polymer. In some embodiments, the polymer can include a total amount of the recurring unit of Formula (II) in the range of about 30 mole % to about 50 mole % based on the ratio of total moles of recurring units of Formula (II) to the total moles of recurring units in the polymer. In some embodiments, the polymer can include a total amount of the recurring unit of Formula (II) in the range of about 10 mole % to about 30 mole % based on the ratio of total moles of recurring units of Formula (II) to the total moles of recurring units in the polymer. In some embodiments, the polymer can include a total amount of the recurring unit of Formula (II) of about 10 mole %, about 20 mole %, about 30 mole %, about 40 mole %, about 50 mole %, about 60 mole % or about 70 mole % based on the ratio of total moles of recurring units of Formula (II) to the total moles of recurring units in the polymer.

In some embodiments, the polymer can include at least about 50 recurring units of Formula (II). In other embodiments, the polymer can include at least about 100 recurring units of Formula (II). In other embodiments, the polymer can include at least about 200 recurring units of Formula (II). In other embodiments, the polymer can include at least about 500 recurring units of Formula (II). In other embodiments, the polymer can include at least about 1000 recurring units of Formula (II). In other embodiments, the polymer can include at least about 1500 recurring units of Formula (II). In other embodiments, the polymer can include about 2000 recurring units of Formula (II).

In some embodiments, the polymer can include from about 50 to about 2000 recurring units of Formula (II). In other embodiments, the polymer can include 200 to about 1500 recurring units of Formula (II). In yet other embodiments, the polymer can include about 300 to about 700 recurring units of Formula (II). In other embodiments, the polymer can include from about 50 to about 100 recurring units, from about 1500 to about 2000 recurring units, from about 1000 to about 1500 recurring units, or from about 700 to about 1000 recurring units of Formula (II). In yet other embodiments, the polymer can include from about 100 to about 200, from about 100 to about 500, or from about 300 to about 600 recurring units of Formula (II).

In some embodiments, the polymer can include ≧90% mole percent of the recurring unit of Formula (III) based on the ratio of total moles of recurring units of Formula (III) to the total moles of recurring units in the polymer. In some embodiments, the polymer can include ≧80% mole percent Of the recurring unit of Formula (III) based on the ratio of total moles of recurring units of Formula (III) to the total moles of recurring units in the polymer. In some embodiments, the polymer can include ≧50% mole percent of the recurring unit of Formula (III) based on the ratio of total moles of recurring units of Formula (III) to the total moles of recurring units in the polymer. In some embodiments, the polymer can include ≧30% mole percent of the recurring unit of Formula (III) based on the ratio of total moles of recurring units of Formula (III) to the total moles of recurring units in the polymer. In some embodiments, the polymer can include ≧15% mole percent of the recurring unit of Formula (III) based on the ratio of total moles of recurring units of Formula (III) to the total moles of recurring units in the polymer. In some embodiments, the polymer can include ≧5% mole percent of the recurring unit of Formula (III) based on the ratio of total moles of recurring units of Formula (III) to the total moles of recurring units in the polymer. In some embodiments, the polymer can include ≧1% mole percent of the recurring unit of Formula (III) based on the ratio of total moles of recurring units of Formula (III) to the total moles of recurring units in the polymer.

In some embodiments, the polymer can include a total amount of the recurring unit of Formula (III) in the range of about 10 mole % to about 90 mole % based on the ratio of total moles of recurring units of Formula (III) to the total moles of recurring units in the polymer. In some embodiments, the polymer can include a total amount of the recurring unit of Formula (III) in the range of about 20 mole % to about 60 mole % based on the ratio of total moles of recurring units of Formula (III) to the total moles of recurring units in the polymer. In some embodiments, the polymer can include a total amount of the recurring unit of Formula (III) in the range of about 30 mole % to about 50 mole % based on the ratio of total moles of recurring units of Formula (III) to the total moles of recurring units in the polymer. In some embodiments, the polymer can include a total amount of the recurring unit of Formula (III) in the range of from about 20 mole % to about 85 mole % based on the ratio of total moles of recurring units of Formula (III) to the total moles of recurring units in the polymer. In some embodiments, the polymer can include a total amount of the recurring unit of Formula (III) in the range of from about 1 mole % to about 20 mole % based on the ratio of total moles of recurring units of Foimula (III) to the total moles of recurring units in the polymer. In some embodiments, the polymer can include a total amount of the recurring unit of Formula (III) of about 10 mole %, about 20 mole %, about 30 mole %, about 40 mole %, about 50 mole %, about 60 mole %, about 70 mole %, about 80 mole %, or about 90 mole % based on the ratio of total moles of recurring units of Formula (III) to the total moles of recurring units in the polymer. In some embodiments, the polymer may not include any recurring units of Formula (III).

In some embodiments, the polymer can include at least about 50 recurring units of Formula (III). In other embodiments, the polymer can include at least about 100 recurring units of Formula (III). In other embodiments, the polymer can include at least about 200 recurring units of Formula (III). In other embodiments, the polymer can include at least about 500 recurring units of Formula (III). In other embodiments, the polymer can include at least about 1000 recurring units of Formula (III). In other embodiments, the polymer can include at least about 1500 recurring units of Formula (III). In other embodiments, the polymer can include about 2000 recurring units of Formula (III).

In some embodiments, the polymer can include from about 50 to about 2000 recurring units of Formula (III). In other embodiments, the polymer can include 200 to about 1500 recurring units of Formula (III). In yet other embodiments, the polymer can include about 300 to about 700 recurring units of Formula (III). In other embodiments, the polymer can include from about 50 to about 100 recurring units, from about 1500 to about 2000 recurring units, from about 1000 to about 1500 recurring units, or from about 700 to about 1000 recurring units of Formula (III). In yet other embodiments, the polymer can include from about 100 to about 200, from about 100 to about 500, or from about 300 to about 600 recurring units of Formula (III).

The relative amounts of the recurring unit of Formula (A) present in the polymer can also vary widely. In some embodiments, the polymer can include ≧90% mole percent of the recurring unit of Formula (A) based on the ratio of total moles of recurring units of Formula (A) to the total moles of recurring units in the polymer. In some embodiments, the polymer can include ≧80% mole percent of the recurring unit of Formula (A) based on the ratio of total moles of recurring units of Formula (A) to the total moles of recurring units in the polymer. In some embodiments, the polymer can include ≧50% mole percent of the recurring unit of Formula (A) based on the ratio of total moles of recurring units of Formula (A) to the total moles of recurring units in the polymer. In some embodiments, the polymer can include ≧30% mole percent of the recurring unit of Formula (A) based on the ratio of total moles of recurring units of Formula (A) to the total moles of recurring units in the polymer. In some embodiments, the polymer can include ≧10% mole percent of the recurring unit of Formula (A) based on the ratio of total moles of recurring units of Formula (A) to the total moles of recurring units in the polymer.

In some embodiments, the polymer can include a total amount of the recurring unit of Formula (A) in the range of about 10 mole % to about 70 mole % based on the ratio of total moles of recurring units of Formula (A) to the total moles of recurring units in the polymer. In some embodiments, the polymer can include a total amount of the recurring unit of Formula (A) in the range of about 20 mole % to about 60 mole % based on the ratio of total moles of recurring units of Formula (A) to the total moles of recurring units in the polymer. In some embodiments, the polymer can include a total amount of the recurring unit of Formula (A) in the range of about 30 mole % to about 50 mole % based on the ratio of total moles of recurring units of Formula (A) to the total moles of recurring units in the polymer. In some embodiments, the polymer can include a total amount of the recurring unit of Formula (A) of about 10 mole %, about 20 mole %, about 30 mole %, about 40 mole %, about 50 mole %, about 60 mole % or about 70 mole % based on the ratio of total moles of recurring units of Formula (A) to the total moles of recurring units in the polymer.

In some embodiments, the polymer can include at least about 50 recurring units of Formula (A). In other embodiments, the polymer can include at least about 100 recurring units of Formula (A). In other embodiments, the polymer can include at least about 200 recurring units of Formula (A). In other embodiments, the polymer can include at least about 500 recurring units of Formula (A). In other embodiments, the polymer can include at least about 1000 recurring units of Formula (A). In other embodiments, the polymer can include at least about 1500 recurring units of Formula (A). In other embodiments, the polymer can include about 2000 recurring units of Formula (A).

In some embodiments, the polymer can include from about 50 to about 2000 recurring units of Formula (A). In other embodiments, the polymer can include 200 to about 1500 recurring units of Formula (A). In yet other embodiments, the polymer can include about 300 to about 700 recurring units of Formula (A). In other embodiments, the polymer can include from about 50 to about 100 recurring units, from about 1500 to about 2000 recurring units, from about 1000 to about 1500 recurring units, or from about 700 to about 1000 recurring units of Formula (A). In yet other embodiments, the polymer can include from about 100 to about 200, from about 100 to about 500, or from about 300 to about 600 recurring units of Formula (A).

In some embodiments, the polymer can include ≧90% mole percent of the recurring unit of Formula (XIII) based on the ratio of total moles of recurring units of Formula (XIII) to the total moles of recurring units in the polymer. In some embodiments, the polymer can include ≧80% mole percent of the recurring unit of Formula (XIII) based on the ratio of total moles of recurring units of Formula (XIII) to the total moles of recurring units in the polymer. In some embodiments, the polymer can include ≧50% mole percent of the recurring unit of Formula (XIII) based on the ratio of total moles of recurring units of Formula (XIII) to the total moles of recurring units in the polymer. In some embodiments, the polymer can include ≧30% mole percent of the recurring unit of Formula (XIII) based on the ratio of total moles of recurring units of Formula (XIII) to the total moles of recurring units in the polymer. In some embodiments, the polymer can include ≧20% mole percent of the recurring unit of Formula (XIII) based on the ratio of total moles of recurring units of Formula (XIII) to the total moles of recurring units in the polymer. In some embodiments, the polymer can include ≧15% mole percent of the recurring unit of Formula (XIII) based on the ratio of total moles of recurring units of Formula (XIII) to the total moles of recurring units in the polymer.

In some embodiments, the polymer can include a total amount of the recurring unit of Formula (XIII) in the range of about 10 mole % to about 90 mole % based on the ratio of total moles of recurring units of Formula (XIII) to the total moles of recurring units in the polymer. In some embodiments, the polymer can include a total amount of the recurring unit of Formula (XIII) in the range of about 20 mole % to about 60 mole % based on the ratio of total moles of recurring units of Formula (XIII) to the total moles of recurring units in the polymer. In some embodiments, the polymer can include a total amount of the recurring unit of Formula (XIII) in the range of about 30 mole % to about 50 mole % based on the ratio of total moles of recurring units of Formula (XIII) to the total moles of recurring units in the polymer. In some embodiments, the polymer can include a total amount of the recurring unit of Formula (XIII) in the range of from about 10 mole % to about 40 mole % based on the ratio of total moles of recurring units of Formula (XIII) to the total moles of recurring units in the polymer. In some embodiments, the polymer can include a total amount of the recurring unit of Formula (XIII) in the range of from about 10 mole % to about 30 mole % based on the ratio of total moles of recurring units of Formula (XIII) to the total moles of recurring units in the polymer. In some embodiments, the polymer can include a total amount of the recurring unit of Formula (XIII) of about 10 mole %, about 20 mole %, about 30 mole %, about 40 mole %, about 50 mole %, about 60 mole %, about 70 mole %, about 80 mole %, or about 90 mole % based on the ratio of total moles of recurring units of Formula (XIII) to the total moles of recurring units in the polymer.

In some embodiments, the polymer can include at least about 50 recurring units of Formula (XIII). In other embodiments, the polymer can include at least about 100 recurring units of Formula (XIII). In other embodiments, the polymer can include at least about 200 recurring units of Formula (XIII). In other embodiments, the polymer can include at least about 500 recurring units of Formula (XIII). In other embodiments, the polymer can include at least about 1000 recurring units of Formula (XIII). In other embodiments, the polymer can include at least about 1500 recurring units of Formula (XIII). In other embodiments, the polymer can include about 2000 recurring units of Formula (XIII).

In some embodiments, the polymer can include from about 50 to about 2000 recurring units of Formula (XIII). In other embodiments, the polymer can include 200 to about 1500 recurring units of Formula (XIII). In yet other embodiments, the polymer can include about 300 to about 700 recurring units of Formula (XIII). In other embodiments, the polymer can include from about 50 to about 100 recurring units, from about 1500 to about 2000 recurring units, from about 1000 to about 1500 recurring units, or from about 700 to about 1000 recurring units of Formula (XIII). In yet other embodiments, the polymer can include from about 100 to about 200, from about 100 to about 500, or from about 300 to about 600 recurring units of Formula (XIII).

In some embodiments directed to a polymer that includes a recurring unit of Formula (I) and a recurring unit of Formula (II), a majority of the recurring units in the polymer are recurring units of Formulae (I) or (II). In some embodiments directed to a polymer that includes a recurring unit of Formula (I) and a recurring unit of Formula (II), at least 50 mole % of the total moles of recurring units in the polymer can be recurring units of Formulae (I) and (II) (e.g., the sum of the moles of recurring units of Formula (I) and moles of recurring units of Formula (II) in the polymer can be equal to at least 50 mole % of the total moles of recurring units in the polymer). In some embodiments directed to a polymer that includes a recurring unit of Formula (I) and a recurring unit of Formula (II), at least 75 mole % of the total moles of recurring units in the polymer can be recurring units of Formulae (I) and (II). In some embodiments directed to a polymer that includes a recurring unit of Formula (I) and a recurring unit of Formula (II), at least 85 mole % of the total moles of recurring units in the polymer can be recurring units of Formulae (I) and (II). In some embodiments directed to a polymer that includes a recurring unit of Formula (I) and a recurring unit of Formula (II), at least 95 mole % of the total moles of recurring units in the polymer can be recurring units of Formulae (I) and (II). In some embodiments directed to a polymer that includes a recurring unit of Formula (I) and a recurring unit of Formula (II), at least 98 mole % of the total moles of recurring units in the polymer can be recurring units of Formulae (I) and (II). In some embodiments directed to a polymer that includes a recurring unit of Formula (I) and a recurring unit of Formula (II), at least 99 mole % of the total moles of recurring units in the polymer can be recurring units of Formulae (I) and (II). In some embodiments directed to a polymer that includes a recurring unit of Formula (I) and a recurring unit of Formula (II), about 100 mole % of the total moles of recurring units in the polymer can be recurring units of Formulae (I) and (II) (e.g., the polymer does not include any recurring units other than the recurring units of Formulae (I) and (II)).

In some embodiments directed to a polymer that includes a recurring unit of Formula (I) and a recurring unit of Formula (II), the polymer can include a total amount of the recurring units of Formulae (I) and (II) in the range of about 50 mole % to about 99 mole % based on the ratio of total moles of recurring units of Formulae (I) and (II) to the total moles of recurring units in the polymer (e.g., the sum of the moles of recurring units of Formula (I) and moles of recurring units of Formula (II) in the polymer can be in the range of about 50 mole % to about 99 mole % based on the total moles of recurring units in the polymer). In some embodiments directed to a polymer that includes a recurring unit of Formula (I) and a recurring unit of Formula (II), the polymer can include a total amount of the recurring units of Formulae (I) and (II) in the range of about 70 mole % to about 98 mole % based on the ratio of total moles of recurring units of Formulae (I) and (II) to the total moles of recurring units in the polymer. In some embodiments directed to a polymer that includes a recurring unit of Formula (I) and a recurring unit of Formula (II), the polymer can include a total amount of the recurring units of Formulae (I) and (II) in the range of about 80 mole % to about 95 mole % based on the ratio of total moles of recurring units of Formulae (I) and (II) to the total moles of recurring units in the polymer. In some embodiments directed to a polymer that includes a recurring unit of Formula (I) and a recurring unit of Formula (II), the polymer can include a total amount of the recurring units of Formulae (I) and (II) in the range of about 90 mole % to about 99 mole % based on the ratio of total moles of recurring units of Formulae (I) and (II) to the total moles of recurring units in the polymer. In some embodiments directed to a polymer that includes a recurring unit of Formula (I) and a recurring unit of Formula (II), the polymer can include a total amount of the recurring units of Formulae (I) and (II) of about 50 mole %, about 60 mole %, about 70 mole %, about 80 mole %, about 90 mole %, about 95 mole %, about 98 mole %, about 99 mole % or about 100 mole % based on the ratio of total moles of recurring units of Formulae (I) and (II) to the total moles of recurring units in the polymer.

In some embodiments directed to a polymer that includes a recurring unit of Formula (I) and a recurring unit of Formula (II), the polymer can include from about 50 mole % to about 99 mole % of recurring units of Formula (I), based on the ratio of total moles of recurring units of Formula (I) to the total moles of recurring units in the polymer; from about 1 mole % to about 50 mole % of recurring units of Formula (II), based on the ratio of total moles of recurring units of Formula (II) to the total moles of recurring units in the polymer; and wherein about 95 mole % to about 99 mole % of the total moles of recurring units in the polymer are recurring units of Formulae (I) or (II). In other embodiments directed to a polymer that includes a recurring unit of Formula (I) and a recurring unit of Formula (II), the polymer can include from about 70 mole % to about 95 mole % of recurring units of Formula (I), based on the ratio of total moles of recurring units of Formula (I) to the total moles of recurring units in the polymer; from about 5 mole % to about 30 mole % of recurring units of Formula (II), based on the ratio of total moles of recurring units of Formula (II) to the total moles of recurring units in the polymer; and wherein about 95 mole % to about 99 mole % of the total moles of recurring units in the polymer are recurring units of Formulae (I) or (II).

In some embodiments directed to a polymer that includes a recurring unit of Formula (I), a recurring unit of Formula (II), and a recurring unit of Formula (III), at least 50 mole % of the total moles of recurring units in the polymer can be recurring units of Formulae (I), (II), and (III) (e.g., the sum of the moles of recurring units of Formula (I), moles of recurring units of Formula (II), and moles of recurring units of Formula (III) in the polymer can be equal to at least 50 mole % of the total moles of recurring units in the polymer). In some embodiments directed to a polymer that includes a recurring unit of Formula (I), a recurring unit of Formula (II), and a recurring unit of Formula (III), at least 75 mole % of the total moles of recurring units in the polymer can be recurring units of Formulae (I), (II), and (III). In some embodiments directed to a polymer that includes a recurring unit of Formula (I), a recurring unit of Formula (II), and a recurring unit of Formula (III), at least 85 mole % of the total moles of recurring units in the polymer can be recurring units of Formulae (I), (II), and (III). In some embodiments directed to a polymer that includes a recurring unit of Formula (I), a recurring unit of Formula (II), and a recurring unit of Formula (III), at least 95 mole % of the total moles of recurring units in the polymer can be recurring units of Formulae (I), (II), and (III). In some embodiments directed to a polymer that includes a recurring unit of Formula (I), a recurring unit of Formula (II), and a recurring unit of Formula (III), at least 98 mole % of the total moles of recurring units in the polymer can be recurring units of Formulae (I), (II), and (III). In some embodiments directed to a polymer that includes a recurring unit of Formula (I), a recurring unit of Formula (II), and a recurring unit of Formula (III), at least 99 mole % of the total moles of recurring units in the polymer can be recurring units of Formulae (I), (II), and (III). In some embodiments directed to a polymer that includes a recurring unit of Formula (I), a recurring unit of Formula (II), and a recurring unit of Formula (III), about 100 mole % of the total moles of recurring units in the polymer can be recurring units of Formulae (I), (II), and (III) (e.g., the polymer may not include any recurring units other than the recurring units of Formulae (I), (II), and (III)).

In some embodiments directed to a polymer that includes a recurring unit of Formula (I), a recurring unit of Formula (II), and a recurring unit of Formula (III), the polymer can include a total amount of the recurring units of Formulae (I), (II), and (III) in the range of from about 50 mole % to about 99 mole % based on the ratio of total moles of recurring units of Formulae (I), (II), and (III) to the total moles of recurring units in the polymer (e.g., the sum of the moles of recurring units of Formula (I), moles of recurring units of Formula (II), and moles of recurring units of Formula (III) in the polymer can be in the range of from about 50 mole % to about 99 mole % based on the total moles of recurring units in the polymer). In some embodiments directed to a polymer that includes a recurring unit of Formula (I), a recurring unit of Formula (II), and a recurring unit of Formula (III), the polymer can include a total amount of the recurring units of Formulae (I), (II), and (III) in the range of about 70 mole % to about 98 mole % based on the ratio of total moles of recurring units of Formulae (I), (II), and (III) to the total moles of recurring units in the polymer. In some embodiments directed to a polymer that includes a recurring unit of Formula (I), a recurring unit of Formula (II), and a recurring unit of Formula (III), the polymer can include a total amount of the recurring units of Formulae (I), (II), and (III) in the range of about 80 mole % to about 95 mole % based on the ratio of total moles of recurring units of Formulae (I), (II), and (III) to the total moles of recurring units in the polymer. In some embodiments directed to a polymer that includes a recurring unit of Formula (I), a recurring unit of Formula (II), and a recurring unit of Formula (III), the polymer can include a total amount of the recurring units of Formulae (I), (II), and (III) in the range of about 90 mole % to about 99 mole % based on the ratio of total moles of recurring units of Formulae (I), (II), and (III) to the total moles of recurring units in the polymer. In some embodiments directed to a polymer that includes a recurring unit of Formula (I), a recurring unit of Formula (II), and a recurring unit of Formula (III), the polymer can include a total amount of the recurring units of Formulae (I), (II), and (III) of about 50 mole %, about 60 mole %, about 70 mole %, about 80 mole %, about 90 mole %, about 95 mole %, about 98 mole %, about 99 mole % or about 100 mole % based on the ratio of total moles of recurring units of Formulae (I), (II), and (III) to the total moles of recurring units in the polymer.

In some embodiments directed to a polymer that includes a recurring unit of Formula (I) a recurring unit of Formula (II), and a recurring unit of Formula (III), the polymer can include from about 10 mole % to about 85 mole % of recurring units of Formula (I), based on the ratio of total moles of recurring units of Formula (I) to the total moles of recurring units in the polymer; from about 1 mole % to about 10 mole % of recurring units of Formula (II), based on the ratio of total moles of recurring units of Formula (II) to the total moles of recurring units in the polymer; and from about 10 mole % to about 85 mole % of recurring units of Formula (III), based on the ratio of total moles of recurring units of Formula (III) to the total moles of recurring units in the polymer; and wherein about 95 mole % to about 99 mole % of the total moles of recurring units in the polymer are recurring units of Formulae (I), (II), or (III). In other embodiments directed to a polymer that includes a recurring unit of Formula (I) a recurring unit of Formula (II), and a recurring unit of Formula (III), the polymer can include from about 45 mole % to about 70 mole % of recurring units of Formula (I), based on the ratio of total moles of recurring units of Formula (I) to the total moles of recurring units in the polymer; from about 1 mole % to about 10 mole % of recurring units of Formula (II), based on the ratio of total moles of recurring units of Formula (II) to the total moles of recurring units in the polymer; and from about 25 mole % to about 50 mole % of recurring units of Formula (III), based on the ratio of total moles of recurring units of Formula (III) to the total moles of recurring units in the polymer; and wherein about 95 mole % to about 99 mole % of the total moles of recurring units in the polymer are recurring units of Formulae (I), (II), or (III).

In some embodiments directed to a polymer that includes a recurring unit of Formula (I), a recurring unit of Formula (II), and a recurring unit of Formula (A), at least 50 mole % of the total moles of recurring units in the polymer can be recurring units of Formulae (I), (II), and (A) (e.g., the sum of the moles of recurring units of Formula (I), moles of recurring units of Formula (II), and moles of recurring units of Formula (A) in the polymer can be equal to at least 50 mole % of the total moles of recurring units in the polymer). In some embodiments directed to a polymer that includes a recurring unit of Formula (I), a recurring unit of Formula (II), and a recurring unit of Formula (A), at least 75 mole % of the total moles of recurring units in the polymer can be recurring units of Formulae (I), (II), and (A). In some embodiments directed to a polymer that includes a recurring unit of Formula (I), a recurring unit of Formula (II), and a recurring unit of Formula (A), at least 85 mole % of the total moles of recurring units in the polymer can be recurring units of Formulae (I), (II), and (A). In some embodiments directed to a polymer that includes a recurring unit of Formula (I), a recurring unit of Formula (II), and a recurring unit of Formula (A), at least 95 mole % of the total moles of recurring units in the polymer can be recurring units of Formulae (I), (II), and (A). In some embodiments directed to a polymer that includes a recurring unit of Formula (I), a recurring unit of Formula (II), and a recurring unit of Formula (A), at least 98 mole % of the total moles of recurring units in the polymer can be recurring units of Formulae (I), (II), and (A). In some embodiments directed to a polymer that includes a recurring unit of Formula (I), a recurring unit of Formula (II), and a recurring unit of Formula (A), at least 99 mole % of the total moles of recurring units in the polymer can be recurring units of Formulae (I), (II), and (A). In some embodiments directed to a polymer that includes a recurring unit of Formula (I), a recurring unit of Formula (II), and a recurring unit of Formula (A), about 100 mole % of the total moles of recurring units in the polymer can be recurring units of Formulae (I), (II), and (A) (e.g., the polymer may not include any recurring units other than the recurring units of Formulae (I), (II), and (A)).

In some embodiments directed to a polymer that includes a recurring unit of Formula (I), a recurring unit of Formula (II), and a recurring unit of Formula (A), the polymer can include a total amount of the recurring units of Formulae (I), (II), and (A) in the range of from about 50 mole % to about 99 mole % based on the ratio of total moles of recurring units of Formulae (I), (II), and (A) to the total moles of recurring units in the polymer (e.g., the sum of the moles of recurring units of Formula (I), moles of recurring units of Formula (II), and moles of recurring units of Formula (A) in the polymer can be in the range of from about 50 mole % to about 99 mole % based on the total moles of recurring units in the polymer). In some embodiments directed to a polymer that includes a recurring unit of Formula (I), a recurring unit of Formula (II), and a recurring unit of Formula (A), the polymer can include a total amount of the recurring units of Formulae (I), (II), and (A) in the range of about 70 mole % to about 98 mole % based on the ratio of total moles of recurring units of Formulae (I), (II), and (A) to the total moles of recurring units in the polymer. In some embodiments directed to a polymer that includes a recurring unit of Formula (I), a recurring unit of Formula (II), and a recurring unit of Formula (A), the polymer can include a total amount of the recurring units of Formulae (I), (II), and (A) in the range of about 80 mole % to about 95 mole % based on the ratio of total moles of recurring units of Formulae (I), (II), and (A) to the total moles of recurring units in the polymer. In some embodiments directed to a polymer that includes a recurring unit of Formula (I), a recurring unit of Formula (II), and a recurring unit of Formula (A), the polymer can include a total amount of the recurring units of Formulae (I), (II), and (A) in the range of about 90 mole % to about 99 mole % based on the ratio of total moles of recurring units of Formulae (I), (II), and (A) to the total moles of recurring units in the polymer. In some embodiments directed to a polymer that includes a recurring unit of Formula (I), a recurring unit of Formula (II), and a recurring unit of Formula (A), the polymer can include a total amount of the recurring units of Formulae (I), (II), and (A) of about 50 mole %, about 60 mole %, about 70 mole %, about 80 mole %, about 90 mole %, about 95 mole %, about 98 mole %, about 99 mole % or about 100 mole % based on the ratio of total moles of recurring units of Formulae (I), (II), and (A) to the total moles of recurring units in the polymer.

In some embodiments directed to a polymer that includes a recurring unit of Formula (I) a recurring unit of Formula (II), and a recurring unit of Formula (A), the polymer can include from about 10 mole % to about 85 mole % of recurring units of Formula (I), based on the ratio of total moles of recurring units of Formula (I) to the total moles of recurring units in the polymer; from about 1 mole % to about 10 mole % of recurring units of Formula (II), based on the ratio of total moles of recurring units of Formula (II) to the total moles of recurring units in the polymer; and from about 10 mole % to about 85 mole % of recurring units of Formula (A), based on the ratio of total moles of recurring units of Formula (A) to the total moles of recurring units in the polymer; and wherein about 95 mole % to about 99 mole % of the total moles of recurring units in the polymer are recurring units of Formulae (I), (II), or (A). In other embodiments directed to a polymer that includes a recurring unit of Formula (I) a recurring unit of Formula (II), and a recurring unit of Formula (A), the polymer can include from about 45 mole % to about 70 mole % of recurring units of Formula (I), based on the ratio of total moles of recurring units of Formula (I) to the total moles of recurring units in the polymer; from about 1 mole % to about 10 mole % of recurring units of Formula (II), based on the ratio of total moles of recurring units of Formula (II) to the total moles of recurring units in the polymer; and from about 25 mole % to about 50 mole % of recurring units of Formula (A), based on the ratio of total moles of recurring units of Formula (A) to the total moles of recurring units in the polymer; and wherein about 95 mole % to about 99 mole % of the total moles of recurring units in the polymer are recurring units of Formulae (I), (II), or (A).

In some embodiments directed to a polymer that includes a recurring unit of Formula (I), a recurring unit of Formula (II), and a recurring unit of Formula (XIII), at least 50 mole % of the total moles of recurring units in the polymer can be recurring units of Formulae (I), (II), and (XIII) (e.g., the sum of the moles of recurring units of Formula (I), moles of recurring units of Formula (II), and moles of recurring units of Formula (XIII) in the polymer can be equal to at least 50 mole % of the total moles of recurring units in the polymer). In some embodiments directed to a polymer that includes a recurring unit of Formula (I), a recurring unit of Formula (II), and a recurring unit of Formula (XIII), at least 75 mole % of the total moles of recurring units in the polymer can be recurring units of Formulae (I), (II), and (XIII). In some embodiments directed to a polymer that includes a recurring unit of Formula (I), a recurring unit of Formula (II), and a recurring unit of Formula (XIII), at least 85 mole % of the total moles of recurring units in the polymer can be recurring units of Formulae (I), (II), and (XIII). In some embodiments directed to a polymer that includes a recurring unit of Formula (I), a recurring unit of Formula (II), and a recurring unit of Formula (XIII), at least 95 mole % of the total moles of recurring units in the polymer can be recurring units of Formulae (I), (II), and (XIII). In some embodiments directed to a polymer that includes a recurring unit of Formula (I), a recurring unit of Formula (II), and a recurring unit of Formula (XIII), at least 98 mole % of the total moles of recurring units in the polymer can be recurring units of Formulae (I), (II), and (XIII). In some embodiments directed to a polymer that includes a recurring unit of Formula (I), a recurring unit of Formula (II), and a recurring unit of Formula (XIII), at least 99 mole % of the total moles of recurring units in the polymer can be recurring units of Formulae (I), (II), and (XIII). In some embodiments directed to a polymer that includes a recurring unit of Formula (I), a recurring unit of Formula (II), and a recurring unit of Formula (XIII), about 100 mole % of the total moles of recurring units in the polymer can be recurring units of Formulae (I), (II), and (XIII) (e.g., the polymer may not include any recurring units other than the recurring units of Formulae (I), (II), and (XIII)).

In some embodiments directed to a polymer that includes a recurring unit of Formula (I), a recurring unit of Formula (II), and a recurring unit of Formula (XIII), the polymer can include a total amount of the recurring units of Formulae (I), (II), and (XIII) in the range of from about 50 mole % to about 99 mole % based on the ratio of total moles of recurring units of Formulae (I), (II), and (XIII) to the total moles of recurring units in the polymer (e.g., the sum of the moles of recurring units of Formula (I), moles of recurring units of Formula (II), and moles of recurring units of Formula (XIII) in the polymer can be in the range of from about 50 mole % to about 99 mole % based on the total moles of recurring units in the polymer). In some embodiments directed to a polymer that includes a recurring unit of Formula (I), a recurring unit of Formula (II), and a recurring unit of Formula (XIII), the polymer can include a total amount of the recurring units of Formulae (I), (II), and (XIII) in the range of about 70 mole % to about 98 mole % based on the ratio of total moles of recurring units of Formulae (I), (II), and (XIII) to the total moles of recurring units in the polymer. In some embodiments directed to a polymer that includes a recurring unit of Formula (I), a recurring unit of Formula (II), and a recurring unit of Formula (XIII), the polymer can include a total amount of the recurring units of Formulae (I), (II), and (XIII) in the range of about 80 mole % to about 95 mole % based on the ratio of total moles of recurring units of Formulae (I), (II), and (XIII) to the total moles of recurring units in the polymer. In some embodiments directed to a polymer that includes a recurring unit of Formula (I), a recurring unit of Formula (II), and a recurring unit of Formula (XIII), the polymer can include a total amount of the recurring units of Formulae (I), (II), and (XIII) in the range of about 90 mole % to about 99 mole % based on the ratio of total moles of recurring units of Formulae (I), (II), and (XIII) to the total moles of recurring units in the polymer. In some embodiments directed to a polymer that includes a recurring unit of Formula (I), a recurring unit of Formula (II), and a recurring unit of Formula (XIII), the polymer can include a total amount of the recurring units of Formulae (I), (II), and (XIII) of about 50 mole %, about 60 mole %, about 70 mole %, about 80 mole %, about 90 mole %, about 95 mole %, about 98 mole %, about 99 mole % or about 100 mole % based on the ratio of total moles of recurring units of Formulae (I), (II), and (XIII) to the total moles of recurring units in the polymer.

In some embodiments directed to a polymer that includes a recurring unit of Formula (I) a recurring unit of Formula (II), and a recurring unit of Formula (XIII), the polymer can include from about 10 mole % to about 85 mole % of recurring units of Formula (I), based on the ratio of total moles of recurring units of Formula (I) to the total moles of recurring units in the polymer; from about 1 mole % to about 10 mole % of recurring units of Formula (II), based on the ratio of total moles of recurring units of Formula (II) to the total moles of recurring units in the polymer; and from about 1 mole % to about 50 mole % of recurring units of Formula (XIII), based on the ratio of total moles of recurring units of Formula (XIII) to the total moles of recurring units in the polymer; and wherein about 95 mole % to about 99 mole % of the total moles of recurring units in the polymer are recurring units of Formulae (I), (II), or (XIII). In other embodiments directed to a polymer that includes a recurring unit of Formula (I) a recurring unit of Formula (II), and a recurring unit of Formula (XIII), the polymer can include from about 45 mole % to about 70 mole % of recurring units of Formula (I), based on the ratio of total moles of recurring units of Formula (I) to the total moles of recurring units in the polymer; from about 1 mole % to about 10 mole % of recurring units of Formula (II), based on the ratio of total moles of recurring units of Formula (II) to the total moles of recurring units in the polymer; and from about 10 mole % to about 30 mole % of recurring units of Formula (XIII), based on the ratio of total moles of recurring units of Formula (XIII) to the total moles of recurring units in the polymer; and wherein about 95 mole % to about 99 mole % of the total moles of recurring units in the polymer are recurring units of Formulae (I), (II), or (XIII).

In some embodiments directed to a polymer that includes a recurring unit of Formula (I), a recurring unit of Formula (II), a recurring unit of Formula (III), and a recurring unit of Formula (A), at least 50 mole % of the total moles of recurring units in the polymer can be recurring units of Formulae (I), (II), (III), and (A) (e.g., the sum of the moles of recurring units of Formula (I), moles of recurring units of Formula (II), moles of recurring units of Formula (III), and moles of recurring units of Formula (A) in the polymer can be equal to at least 50 mole % of the total moles of recurring units in the polymer). In some embodiments directed to a polymer that includes a recurring unit of Formula (I), a recurring unit of Formula (II), a recurring unit of Formula (III), and a recurring unit of Formula (A), at least 75 mole % of the total moles of recurring units in the polymer can be recurring units of Formulae (I), (II), (III), and (A). In some embodiments directed to a polymer that includes a recurring unit of Formula (I), a recurring unit of Formula (II), a recurring unit of Formula (III), and a recurring unit of Formula (A), at least 85 mole % of the total moles of recurring units in the polymer can be recurring units of Formulae (I), (II), (III), and (A). In some embodiments directed to a polymer that includes a recurring unit of Formula (I), a recurring unit of Formula (II), a recurring unit of Formula (III), and a recurring unit of Formula (A), at least 95 mole % of the total moles of recurring units in the polymer can be recurring units of Formulae (I), (II), (III), and (A). In some embodiments directed to a polymer that includes a recurring unit of Formula (I), a recurring unit of Formula (II), a recurring unit of Formula (III), and a recurring unit of Formula (A), at least 98 mole % of the total moles of recurring units in the polymer can be recurring units of Formulae (I), (II), (III), and (A). In some embodiments directed to a polymer that includes a recurring unit of Formula (I), a recurring unit of Formula (II), a recurring unit of Formula (III), and a recurring unit of Formula (A), at least 99 mole % of the total moles of recurring units in the polymer can be recurring units of Formulae (I), (II), (III), and (A). In some embodiments directed to a polymer that includes a recurring unit of Formula (I), a recurring unit of Formula (II), a recurring unit of Formula (III), and a recurring unit of Formula (A), about 100 mole % of the total moles of recurring units in the polymer can be recurring units of Formulae (I), (II), (III), and (A) (e.g., the polymer may not include any recurring units other than the recurring units of Formulae (I), (II), (III), and (A)).

In some embodiments directed to a polymer that includes a recurring unit of Formula (I), a recurring unit of Formula (II), a recurring unit of Formula (III), and a recurring unit of Formula (A), the polymer can include a total amount of the recurring units of Formulae (I), (II), (III), and (A) in the range of from about 50 mole % to about 99 mole % based on the ratio of total moles of recurring units of Formulae (I), (II), (III), and (A) to the total moles of recurring units in the polymer (e.g., the sum of the moles of recurring units of Formula (I), moles of recurring units of Formula (II), moles of recurring units of Formula (III), and moles of recurring units of Formula (A) in the polymer can be in the range of from about 50 mole % to about 99 mole % based on the total moles of recurring units in the polymer). In some embodiments directed to a polymer that includes a recurring unit of Formula (I), a recurring unit of Formula (II), a recurring unit of Formula (III), and a recurring unit of Formula (A), the polymer can include a total amount of the recurring units of Formulae (I), (II), (III), and (A) in the range of about 70 mole % to about 98 mole % based on the ratio of total moles of recurring units of Formulae (I), (II), (III), and (A) to the total moles of recurring units in the polymer. In some embodiments directed to a polymer that includes a recurring unit of Formula (I), a recurring unit of Formula (II), a recurring unit of Formula (III), and a recurring unit of Formula (A), the polymer can include a total amount of the recurring units of Formulae (I), (II), (III), and (A) in the range of about 80 mole % to about 95 mole % based on the ratio of total moles of recurring units of Formulae (I), (II), (III), and (A) to the total moles of recurring units in the polymer. In some embodiments directed to a polymer that includes a recurring unit of Formula (I), a recurring unit of Formula (II), a recurring unit of Formula (III), and a recurring unit of Formula (A), the polymer can include a total amount of the recurring units of Formulae (I), (II), (III), and (A) in the range of about 90 mole % to about 99 mole % based on the ratio of total moles of recurring units of Formulae (I), (II), (III), and (A) to the total moles of recurring units in the polymer. In some embodiments directed to a polymer that includes a recurring unit of Formula (I), a recurring unit of Formula (II), a recurring unit of Formula (III), and a recurring unit of Formula (A), the polymer can include a total amount of the recurring units of Formulae (I), (II), (III), and (A) of about 50 mole %, about 60 mole %, about 70 mole %, about 80 mole %, about 90 mole %, about 95 mole %, about 98 mole %, about 99 mole % or about 100 mole % based on the ratio of total moles of recurring units of Formulae (I), (II), (III), and (A) to the total moles of recurring units in the polymer.

In some embodiments directed to a polymer that includes a recurring unit of Formula (I), a recurring unit of Formula (II), a recurring unit of Formula (III), and a recurring unit of Formula (A), the polymer can include from about 10 mole % to about 85 mole % of recurring units of Formula (I), based on the ratio of total moles of recurring units of Formula (I) to the total moles of recurring units in the polymer; from about 1 mole % to about 10 mole % of recurring units of Formula (II), based on the ratio of total moles of recurring units of Formula (II) to the total moles of recurring units in the polymer; from about 10 mole % to about 85 mole % of recurring units of Formula (III), based on the ratio of total moles of recurring units of Formula (III) to the total moles of recurring units in the polymer; from about 1 mole % to about 50 mole % of recurring units of Formula (A), based on the ratio of total moles of recurring units of Formula (A) to the total moles of recurring units in the polymer; and wherein about 95 mole % to about 99 mole % of the total moles of recurring units in the polymer are recurring units of Formulae (I), (II), (III), or (A). In other embodiments directed to a polymer that includes a recurring unit of Formula (I) a recurring unit of Formula (II), a recurring unit of Formula (III), and a recurring unit of Formula (A), the polymer can include from about 45 mole % to about 70 mole % of recurring units of Formula (I), based on the ratio of total moles of recurring units of Formula (I) to the total moles of recurring units in the polymer; from about 1 mole % to about 10 mole % of recurring units of Formula (II), based on the ratio of total moles of recurring units of Formula (II) to the total moles of recurring units in the polymer; from about 25 mole % to about 50 mole % of recurring units of Formula (III), based on the ratio of total moles of recurring units of Formula (III) to the total moles of recurring units in the polymer; and from about 1 mole % to about 25 mole % of recurring units of Formula (A), based on the ratio of total moles of recurring units of Formula (A) to the total moles of recurring units in the polymer; and wherein about 95 mole % to about 99 mole % of the total moles of recurring units in the polymer are recurring units of Formulae (I), (II), (III), or (A).

In some embodiments directed to a polymer that includes a recurring unit of Formula (I), a recurring unit of Formula (II), a recurring unit of Formula (III), and a recurring unit of Formula (XIII), at least 50 mole % of the total moles of recurring units in the polymer can be recurring units of Formulae (I), (II), (III), and (XIII) (e.g., the sum of the moles of recurring units of Formula (I), moles of recurring units of Formula (II), moles of recurring units of Formula (III), and moles of recurring units of Formula (XIII) in the polymer can be equal to at least 50 mole % of the total moles of recurring units in the polymer). In some embodiments directed to a polymer that includes a recurring unit of Formula (I), a recurring unit of Formula (II), a recurring unit of Formula (III), and a recurring unit of Formula (XIII), at least 75 mole % of the total moles of recurring units in the polymer can be recurring units of Formulae (I), (II), (III), and (XIII). In some embodiments directed to a polymer that includes a recurring unit of Formula (I), a recurring unit of Formula (II), a recurring unit of Formula (III), and a recurring unit of Formula (XIII), at least 85 mole % of the total moles of recurring units in the polymer can be recurring units of Formulae (I), (II), (III), and (XIII). In some embodiments directed to a polymer that includes a recurring unit of Formula (I), a recurring unit of Formula (II), a recurring unit of Formula (III), and a recurring unit of Formula (XIII), at least 95 mole % of the total moles of recurring units in the polymer can be recurring units of Formulae (I), (II), (III), and (XIII). In some embodiments directed to a polymer that includes a recurring unit of Formula (I), a recurring unit of Formula (II), a recurring unit of Formula (III), and a recurring unit of Formula (XIII), at least 98 mole % of the total moles of recurring units in the polymer can be recurring units of Formulae (I), (II), (III), and (XIII). In some embodiments directed to a polymer that includes a recurring unit of Formula (I), a recurring unit of Formula (II), a recurring unit of Formula (III), and a recurring unit of Formula (XIII), at least 99 mole % of the total moles of recurring units in the polymer can be recurring units of Formulae (I), (II), (III), and (XIII). In some embodiments directed to a polymer that includes a recurring unit of Formula (I), a recurring unit of Formula (II), a recurring unit of Formula (III), and a recurring unit of Formula (XIII), about 100 mole % of the total moles of recurring units in the polymer can be recurring units of Formulae (I), (II), (III), and (XIII) (e.g., the polymer may not include any recurring units other than the recurring units of Formulae (I), (II), (III), and (XIII)).

In some embodiments directed to a polymer that includes a recurring unit of Formula (I), a recurring unit of Formula (II), a recurring unit of Formula (III), and a recurring unit of Formula (XIII), the polymer can include a total amount of the recurring units of Formulae (I), (II), (III), and (XIII) in the range of from about 50 mole % to about 99 mole % based on the ratio of total moles of recurring units of Formulae (I), (II), (III), and (XIII) to the total moles of recurring units in the polymer (e.g., the sum of the moles of recurring units of Formula (I), moles of recurring units of Formula (II), moles of recurring units of Formula (III), and moles of recurring units of Formula (XIII) in the polymer can be in the range of from about 50 mole % to about 99 mole % based on the total moles of recurring units in the polymer). In some embodiments directed to a polymer that includes a recurring unit of Formula (I), a recurring unit of Formula (II), a recurring unit of Formula (III), and a recurring unit of Formula (XIII), the polymer can include a total amount of the recurring units of Formulae (I), (II), (III), and (XIII) in the range of about 70 mole % to about 98 mole % based on the ratio of total moles of recurring units of Formulae (I), (II), (III), and (XIII) to the total moles of recurring units in the polymer. In some embodiments directed to a polymer that includes a recurring unit of Formula (I), a recurring unit of Formula (II), a recurring unit of Formula (III), and a recurring unit of Formula (XIII), the polymer can include a total amount of the recurring units of Formulae (I), (II), (III), and (XIII) in the range of about 80 mole % to about 95 mole % based on the ratio of total moles of recurring units of Formulae (I), (II), (III), and (XIII) to the total moles of recurring units in the polymer. In some embodiments directed to a polymer that includes a recurring unit of Formula (I), a recurring unit of Formula (II), a recurring unit of Formula (III), and a recurring unit of Formula (XIII), the polymer can include a total amount of the recurring units of Formulae (I), (II), (III), and (XIII) in the range of about 90 mole % to about 99 mole % based on the ratio of total moles of recurring units of Formulae (I), (II), (III), and (XIII) to the total moles of recurring units in the polymer. In some embodiments directed to a polymer that includes a recurring unit of Formula (I), a recurring unit of Formula (II), a recurring unit of Formula (III), and a recurring unit of Formula (XIII), the polymer can include a total amount of the recurring units of Formulae (I), (II), (III), and (XIII) of about 50 mole %, about 60 mole %, about 70 mole %, about 80 mole %, about 90 mole %, about 95 mole %, about 98 mole %, about 99 mole % or about 100 mole % based on the ratio of total moles of recurring units of Formulae (I), (II), (III), and (XIII) to the total moles of recurring units in the polymer.

In some embodiments directed to a polymer that includes a recurring unit of Formula (I), a recurring unit of Formula (II), a recurring unit of Formula (III), and a recurring unit of Formula (XIII), the polymer can include from about 10 mole % to about 85 mole % of recurring units of Formula (I), based on the ratio of total moles of recurring units of Formula (I) to the total moles of recurring units in the polymer; from about 1 mole % to about 10 mole % of recurring units of Formula (II), based on the ratio of total moles of recurring units of Formula (II) to the total moles of recurring units in the polymer; from about 10 mole % to about 85 mole % of recurring units of Formula (III), based on the ratio of total moles of recurring units of Formula (III) to the total moles of recurring units in the polymer; from about 1 mole % to about 50 mole % of recurring units of Formula (XIII), based on the ratio of total moles of recurring units of Formula (XIII) to the total moles of recurring units in the polymer; and wherein about 95 mole % to about 99 mole % of the total moles of recurring units in the polymer are recurring units of Formulae (I), (II), (III), or (XIII). In other embodiments directed to a polymer that includes a recurring unit of Formula (I) a recurring unit of Formula (II), a recurring unit of Formula (III), and a recurring unit of Formula (XIII), the polymer can include from about 45 mole % to about 70 mole % of recurring units of Formula (I), based on the ratio of total moles of recurring units of Formula (I) to the total moles of recurring units in the polymer; from about 1 mole % to about 10 mole % of recurring units of Formula (II), based on the ratio of total moles of recurring units of Formula (II) to the total moles of recurring units in the polymer; from about 25 mole % to about 50 mole % of recurring units of Formula (III), based on the ratio of total moles of recurring units of Formula (III) to the total moles of recurring units in the polymer; and from about 1 mole % to about 25 mole % of recurring units of Formula (XIII), based on the ratio of total moles of recurring units of Formula (XIII) to the total moles of recurring units in the polymer; and wherein about 95 mole % to about 99 mole % of the total moles of recurring units in the polymer are recurring units of Formulae (I), (II), (III), or (XIII).

In some embodiments directed to a polymer that includes a recurring unit of Formula (I), a recurring unit of Formula (II), a recurring unit of Formula (A), and a recurring unit of Formula (XIII), at least 50 mole % of the total moles of recurring units in the polymer can be recurring units of Formulae (I), (II), (A), and (XIII) (e.g., the sum of the moles of recurring units of Formula (I), moles of recurring units of Formula (II), moles of recurring units of Formula (A), and moles of recurring units of Formula (XIII) in the polymer can be equal to at least 50 mole % of the total moles of recurring units in the polymer). In some embodiments directed to a polymer that includes a recurring unit of Formula (I), a recurring unit of Formula (II), a recurring unit of Formula (A), and a recurring unit of Formula (XIII), at least 75 mole % of the total moles of recurring units in the polymer can be recurring units of Formulae (I), (II), (A), and (XIII). In some embodiments directed to a polymer that includes a recurring unit of Formula (I), a recurring unit of Formula (II), a recurring unit of Formula (A), and a recurring unit of Formula (XIII), at least 85 mole % of the total moles of recurring units in the polymer can be recurring units of Formulae (I), (II), (A), and (XIII). In some embodiments directed to a polymer that includes a recurring unit of Formula (I), a recurring unit of Formula (II), a recurring unit of Formula (A), and a recurring unit of Formula (XIII), at least 95 mole % of the total moles of recurring units in the polymer can be recurring units of Formulae (I), (II), (A), and (XIII). In some embodiments directed to a polymer that includes a recurring unit of Formula (I), a recurring unit of Formula (II), a recurring unit of Formula (A), and a recurring unit of Formula (XIII), at least 98 mole % of the total moles of recurring units in the polymer can be recurring units of Formulae (I), (II), (A), and (XIII). In some embodiments directed to a polymer that includes a recurring unit of Formula (I), a recurring unit of Formula (II), a recurring unit of Formula (A), and a recurring unit of Formula (XIII), at least 99 mole % of the total moles of recurring units in the polymer can be recurring units of Formulae (I), (II), (A), and (XIII). In some embodiments directed to a polymer that includes a recurring unit of Formula (I), a recurring unit of Formula (II), a recurring unit of Formula (A), and a recurring unit of Formula (XIII), about 100 mole % of the total moles of recurring units in the polymer can be recurring units of Formulae (I), (II), (A), and (XIII) (e.g., the polymer may not include any recurring units other than the recurring units of Formulae (I), (II), (A), and (XIII)).

In some embodiments directed to a polymer that includes a recurring unit of Formula (I), a recurring unit of Formula (II), a recurring unit of Formula (A), and a recurring unit of Formula (XIII), the polymer can include a total amount of the recurring units of Formulae (I), (II), (A), and (XIII) in the range of from about 50 mole % to about 99 mole % based on the ratio of total moles of recurring units of Formulae (I), (II), (A), and (XIII) to the total moles of recurring units in the polymer (e.g., the sum of the moles of recurring units of Formula (I), moles of recurring units of Formula (II), moles of recurring units of Formula (A), and moles of recurring units of Formula (XIII) in the polymer can be in the range of from about 50 mole % to about 99 mole % based on the total moles of recurring units in the polymer). In some embodiments directed to a polymer that includes a recurring unit of Formula (I), a recurring unit of Formula (II), a recurring unit of Formula (A), and a recurring unit of Formula (XIII), the polymer can include a total amount of the recurring units of Formulae (I), (II), (A), and (XIII) in the range of about 70 mole % to about 98 mole % based on the ratio of total moles of recurring units of Formulae (I), (II), (A), and (XIII) to the total moles of recurring units in the polymer. In some embodiments directed to a polymer that includes a recurring unit of Formula (I), a recurring unit of Formula (II), a recurring unit of Formula (A), and a recurring unit of Formula (XIII), the polymer can include a total amount of the recurring units of Formulae (I), (II), (A), and (XIII) in the range of about 80 mole % to about 95 mole % based on the ratio of total moles of recurring units of Formulae (I), (II), (A), and (XIII) to the total moles of recurring units in the polymer. In some embodiments directed to a polymer that includes a recurring unit of Formula (I), a recurring unit of Formula (II), a recurring unit of Formula (A), and a recurring unit of Formula (XIII), the polymer can include a total amount of the recurring units of Formulae (I), (II), (A), and (XIII) in the range of about 90 mole % to about 99 mole % based on the ratio of total moles of recurring units of Formulae (I), (II), (A), and (XIII) to the total moles of recurring units in the polymer. In some embodiments directed to a polymer that includes a recurring unit of Formula (I), a recurring unit of Formula (II), a recurring unit of Formula (A), and a recurring unit of Formula (XIII), the polymer can include a total amount of the recurring units of Formulae (I), (II), (A), and (XIII) of about 50 mole %, about 60 mole %, about 70 mole %, about 80 mole %, about 90 mole %, about 95 mole %, about 98 mole %, about 99 mole % or about 100 mole % based on the ratio of total moles of recurring units of Formulae (I), (II), (A), and (XIII) to the total moles of recurring units in the polymer.

In some embodiments directed to a polymer that includes a recurring unit of Formula (I), a recurring unit of Formula (II), a recurring unit of Formula (A), and a recurring unit of Formula (XIII), the polymer can include from about 10 mole % to about 85 mole % of recurring units of Formula (I), based on the ratio of total moles of recurring units of Formula (I) to the total moles of recurring units in the polymer; from about 1 mole % to about 10 mole % of recurring units of Formula (II), based on the ratio of total moles of recurring units of Formula (II) to the total moles of recurring units in the polymer; from about 1 mole % to about 50 mole % of recurring units of Formula (A), based on the ratio of total moles of recurring units of Formula (A) to the total moles of recurring units in the polymer; from about 1 mole % to about 50 mole % of recurring units of Formula (XIII), based on the ratio of total moles of recurring units of Formula (XIII) to the total moles of recurring units in the polymer; and wherein about 95 mole % to about 99 mole % of the total moles of recurring units in the polymer are recurring units of Formulae (I), (II), (A), or (XIII). In other embodiments directed to a polymer that includes a recurring unit of Formula (I) a recurring unit of Formula (II), a recurring unit of Formula (A), and a recurring unit of Formula (XIII), the polymer can include from about 45 mole % to about 70 mole % of recurring units of Formula (I), based on the ratio of total moles of recurring units of Formula (I) to the total moles of recurring units in the polymer; from about 1 mole % to about 10 mole % of recurring units of Formula (II), based on the ratio of total moles of recurring units of Formula (II) to the total moles of recurring units in the polymer; from about 1 mole % to about 30 mole % of recurring units of Formula (A), based on the ratio of total moles of recurring units of Formula (A) to the total moles of recurring units in the polymer; and from about 10 mole % to about 30 mole % of recurring units of Formula (XIII), based on the ratio of total moles of recurring units of Formula (XIII) to the total moles of recurring units in the polymer; and wherein about 95 mole % to about 99 mole % of the total moles of recurring units in the polymer are recurring units of Formulae (I), (II), (A), or (XIII).

In some embodiments directed to a polymer that can include a recurring unit of Formula (I), a recurring unit of Formula (II), a recurring unit of Formula (III), a recurring unit of Formula (A), and a recurring unit of Formula (XIII), at least 50 mole % of the total moles of recurring units in the polymer can be recurring units of Formulae (I), (II), (III), (A), and (XIII) (e.g., the sum of the moles of recurring units of Formula (I), moles of recurring units of Formula (II), moles of recurring units of Formula (III), moles of recurring units of Formula (A), and moles of recurring units of Formula (XIII) in the polymer can be equal to at least 50 mole % of the total moles of recurring units in the polymer). In some embodiments directed to a polymer that can include a recurring unit of Formula (I), a recurring unit of Formula (II), a recurring unit of Formula (III), a recurring unit of Formula (A), and a recurring unit of Formula (XIII), at least 75 mole % of the total moles of recurring units in the polymer can be recurring units of Formulae (I), (II), (III), (A), and (XIII). In some embodiments directed to a polymer that can include a recurring unit of Formula (I), a recurring unit of Formula (II), a recurring unit of Formula (III), a recurring unit of Formula (A), and a recurring unit of Formula (XIII), at least 85 mole % of the total moles of recurring units in the polymer can be recurring units of Formulae (I), (II), (III), (A), and (XIII). In some embodiments directed to a polymer that can include a recurring unit of Formula (I), a recurring unit of Formula (II), a recurring unit of Formula (III), a recurring unit of Formula (A), and a recurring unit of Formula (XIII), at least 95 mole % of the total moles of recurring units in the polymer can be recurring units of Formulae (I), (II), (III), (A), and (XIII). In some embodiments directed to a polymer that can include a recurring unit of Formula (I), a recurring unit of Formula (II), a recurring unit of Formula (III), a recurring unit of Formula (A), and a recurring unit of Formula (XIII), at least 98 mole % of the total moles of recurring units in the polymer can be recurring units of Formulae (I), (II), (III), (A), and (XIII). In some embodiments directed to a polymer that can include a recurring unit of Formula (I), a recurring unit of Formula (II), a recurring unit of Formula (III), a recurring unit of Formula (A), and a recurring unit of Formula (XIII), at least 99 mole % of the total moles of recurring units in the polymer can be recurring units of Formulae (I), (II), (III), (A), and (XIII). In some embodiments directed to a polymer that can include a recurring unit of Formula (I), a recurring unit of Formula (II), a recurring unit of Formula (III), a recurring unit of Formula (A), and a recurring unit of Formula (XIII), about 100 mole % of the total moles of recurring units in the polymer can be recurring units of Formulae (I), (II), (III), (A), and (XIII) (e.g., the polymer may not include any recurring units other than the recurring units of Formulae (I), (II), (III), (A), and (XIII)).

In some embodiments directed to a polymer that includes a recurring unit of Formula (I), a recurring unit of Formula (II), a recurring unit of Formula (III), a recurring unit of Formula (A), and a recurring unit of Formula (XIII), the polymer can include a total amount of the recurring units of Formulae (I), (II), (III), (A), and (XIII) in the range of from about 50 mole % to about 99 mole % based on the ratio of total moles of recurring units of Formulae (I), (II), (III), (A), and (XIII) to the total moles of recurring units in the polymer (e.g., the sum of the moles of recurring units of Formula (I), moles of recurring units of Formula (II), moles of recurring units of Formula (III), moles of recurring units of Formula (A), and moles of recurring units of Formula (XIII) in the polymer can be in the range of from about 50 mole % to about 99 mole % based on the total moles of recurring units in the polymer). In some embodiments directed to a polymer that includes a recurring unit of Formula (I), a recurring unit of Formula (II), a recurring unit of Formula (III), a recurring unit of Formula (A), and a recurring unit of Formula (XIII), the polymer can include a total amount of the recurring units of Formulae (I), (II), (III), (A), and (XIII) in the range of about 70 mole % to about 98 mole % based on the ratio of total moles of recurring units of Formulae (I), (II), (III), (A), and (XIII) to the total moles of recurring units in the polymer. In some embodiments directed to a polymer that includes a recurring unit of Formula (I), a recurring unit of Formula (II), a recurring unit of Formula (III), a recurring unit of Formula (A), and a recurring unit of Formula (XIII), the polymer can include a total amount of the recurring units of Formulae (I), (II), (III), (A), and (XIII) in the range of about 80 mole % to about 95 mole % based on the ratio of total moles of recurring units of Formulae (I), (II), (III), (A), and (XIII) to the total moles of recurring units in the polymer. In some embodiments directed to a polymer that includes a recurring unit of Formula (I), a recurring unit of Formula (II), a recurring unit of Formula (III), a recurring unit of Formula (A), and a recurring unit of Formula (XIII), the polymer can include a total amount of the recurring units of Formulae (I), (II), (III), (A), and (XIII) in the range of about 90 mole % to about 99 mole % based on the ratio of total moles of recurring units of Formulae (I), (II), (III), (A), and (XIII) to the total moles of recurring units in the polymer. In some embodiments directed to a polymer that includes a recurring unit of Formula (I), a recurring unit of Formula (II), a recurring unit of Formula (III), a recurring unit of Formula (A), and a recurring unit of Formula (XIII), the polymer can include a total amount of the recurring units of Formulae (I), (II), (III), (A), and (XIII) of about 50 mole %, about 60 mole %, about 70 mole %, about 80 mole %, about 90 mole %, about 95 mole %, about 98 mole %, about 99 mole % or about 100 mole % based on the ratio of total moles of recurring units of Formulae (I), (II), (III), (A), and (XIII) to the total moles of recurring units in the polymer.

In some embodiments directed to a polymer that includes a recurring unit of Formula (I), a recurring unit of Formula (II), a recurring unit of Formula (III), a recurring unit of Formula (A), and a recurring unit of Formula (XIII), the polymer can include from about 10 mole % to about 85 mole % of recurring units of Formula (I), based on the ratio of total moles of recurring units of Formula (I) to the total moles of recurring units in the polymer; from about 1 mole % to about 10 mole % of recurring units of Formula (II), based on the ratio of total moles of recurring units of Formula (II) to the total moles of recurring units in the polymer; from about 10 mole % to about 85 mole % of recurring units of Formula (III), based on the ratio of total moles of recurring units of Formula (III) to the total moles of recurring units in the polymer; from about 1 mole % to about 50 mole % of recurring units of Formula (A), based on the ratio of total moles of recurring units of Formula (A) to the total moles of recurring units in the polymer; from about 1 mole % to about 50 mole % of recurring units of Formula (XIII), based on the ratio of total moles of recurring units of Formula (XIII) to the total moles of recurring units in the polymer; and wherein about 95 mole % to about 99 mole % of the total moles of recurring units in the polymer are recurring units of Formulae (I), (II), (III), (A), or (XIII). In other embodiments directed to a polymer that includes a recurring unit of Formula (I) a recurring unit of Formula (II), a recurring unit of Formula (III), a recurring unit of Formula (A), and a recurring unit of Formula (XIII), the polymer can include from about 45 mole % to about 70 mole % of recurring units of Formula (I), based on the ratio of total moles of recurring units of Formula (I) to the total moles of recurring units in the polymer; from about 1 mole % to about 10 mole % of recurring units of Formula (II), based on the ratio of total moles of recurring units of Formula (II) to the total moles of recurring units in the polymer; from about 25 mole % to about 50 mole % of recurring units of Formula (III), based on the ratio of total moles of recurring units of Formula (III) to the total moles of recurring units in the polymer; from about 1 mole % to about 25 mole % of recurring units of Formula (A), based on the ratio of total moles of recurring units of Formula (A) to the total moles of recurring units in the polymer; and from about 10 mole % to about 30 mole % of recurring units of Formula (XIII), based on the ratio of total moles of recurring units of Formula (XIII) to the total moles of recurring units in the polymer; and wherein about 95 mole % to about 99 mole % of the total moles of recurring units in the polymer are recurring units of Formulae (I), (II), (III), (A), or (XIII).

In some embodiments, the polymer can include at least about 55 total recurring units. In other embodiments, the polymer can include at least about 100 total recurring units. In other embodiments, the polymer can include at least about 200 total recurring units. In other embodiments, the polymer can include at least about 500 total recurring units. In other embodiments, the polymer can include at least about 1000 total recurring units. In other embodiments, the polymer can include at least about 1500 total recurring units. In other embodiments, the polymer can include at least about 2000 total recurring units. In other embodiments, the polymer can include at least about 3000 total recurring units. In other embodiments, the polymer can include at least about 4000 total recurring units. In other embodiments, the polymer can include at least about 5000 recurring units.

In some embodiments, the polymer can include from about 55 to about 5000 total recurring units. In other embodiments, the polymer can include from about 100 to about 4000 total recurring units. In yet other embodiments, the polymer can include about 300 to about 700 recurring units. In other embodiments, the polymer can include from about 50 to about 100 recurring units, from about 1500 to about 2000 recurring units, from about 1000 to about 1500 recurring units, or from about 700 to about 1000 recurring units. In yet other embodiments, the polymer can include from about 100 to about 200, from about 100 to about 500, or from about 300 to about 600 recurring units. In still other embodiments, the polymer can include from about 1000 to about 5000, from about 2000 to about 5000, from about 3000 to about 5000, from about 4000 to about 5000, from about 2000 to about 4000, or from about 3000 to about 4000 total recurring units.

In some embodiments, the polymer can have a weight average molecular weight (M_{w}) in the range of from about 8 kDa to about 200 kDa. In other embodiments, the polymer can have a weight average molecular weight in the range of from about 8 kDa to about 150 kDa. In other embodiments, the polymer can have a weight average molecular weight in the range of from about 8 kDa to about 100 kDa. In other embodiments, the polymer can have a weight average molecular weight in the range of from about 10 kDa to about 50 kDa. In other embodiments, the polymer can have a weight average molecular weight in the range of from about 20 kDa to about 45 kDa.

Those skilled in the art also appreciate that the relative mole percentages of the recurring units of Formulae (I), (II), (III), (A), and/or (XIII) can be varied depending on the particular recurring units and/or nucleic acid being incorporated into the polymer. Examples of these properties include, but are not limited to, solubility, degradability, transfection efficiency, and toxicity. In addition, a combination of different recurring units of Formulae (I), (II), (III), and/or (XIII) and/or additional recurring units (e.g., a recurring unit of Formula (A)) having other structures and/or properties may be included in the polymers described herein depending on the desired characteristics of the polymer. The additional recurring units may be selected based on information available to those skilled in the art.

For example, in some embodiments, the polymer can include the recurring unit of Formula (I) in an amount that allows a nucleic acid to be associated with the polymer (e.g., via an electrostatic or covalent bond) at a weight ratio selected from at least 1:50 (e.g., at least 1 g of nucleic acid to 50 g of polymer), 1:40, 1:30, 1:20, 1:10, 1:5, 1:2.5 and 1:1. The amount of polymer relative to the amount of nucleic acid may be expressed as an amino nitrogen to phosphate ratio (N/P), where N represents the number of terminal amino groups from the polymer and P represents the number of phosphate groups from the nucleic acid. In some embodiments, the polymer can include a recurring unit of Formula (I) in an amount that allows the nucleic acid to be associated with the polymer (e.g., via an electrostatic or covalent bond) at a N/P ratio selected from at least 1:1 (e.g., at least 1 amino group from the polymer to 1 phosphate group from the nucleic acid), 4:1, 8:1, 12:1, 16:1, and 20:1.

In other embodiments, the polymer can include the recurring unit of Formula (II) in an amount that is effective to give the polymer sufficient lipophilicity to provide a desired degree of cell wall penetration (e.g., disruption of the endosomal membrane) and/or phagocytosis. In yet another example, the polymer can include the recurring unit of Formula (II) in an amount that provides an increased degree of cell wall penetration (as measured, e.g., via a cell transfection assay) as compared to an otherwise similar polymer lacking a recurring unit of Formula (II) (e.g., a homopolymer having recurring units of Formula (I)). In another example, the polymer can include a recurring unit of Formula (III) in an amount that reduces toxicity and/or increases biocompatibility of the polymer as a whole, as compared to an otherwise similar polymer that lacks a recurring unit of Formula (III). In another example, the polymer can include a recurring unit of Formula (A) in an amount that reduces toxicity and/or increases biocompatibility of the polymer as a whole, as compared to an otherwise similar polymer that lacks a recurring unit of Formula (A).

As described herein, the recurring unit of Formula (XIII) can include a group that has a pH transition point which can affect one or more properties of the overall polymer. Those skilled in the art may appreciate that hydrophilicity and/or hydrophobicity of R²⁰ can affect the solubility of the polymer in a particular solvent. Thus, in some embodiments, the polymer can include an amount of the recurring unit of Formula (XIII) that is sufficient to affect and/or alter the solubility of the overall polymer.

For example, the polymer can include an amount of the recurring unit of Formula (XIII) that makes the polymer relatively less soluble at a first pH and relatively more soluble at a second pH. In some embodiments, an amount of the polymer can be more insoluble in a solvent at a pH less than the transition point compared to the same amount of the same polymer in the same solvent at a pH greater than or equal to the transition point. The solubility can be measured according to any indicia known to those skilled in the art, such as turbidity. The solvent can be any solvent, such as blood or other aqueous solvents.

As described herein, in some embodiments the transition point can be a pH that is generally equal to the pH of the micro-environment of tumor tissue. In these embodiments, the group having a pH transition point (e.g., a recurring unit of Formula (XIII)) can be hydrophilic at a pH generally greater than or equal to the pH transition point and hydrophobic at a pH generally less than the pH transition point. Advantageously, the polymer can include an amount of the recurring unit of Formula (XIII) that makes the polymer more soluble in blood that is outside the micro-environment of tumor tissue, and more insoluble in blood that is within the micro-environment of tumor tissue. In these embodiments, when the polymer is more hydrophobic and/or insoluble in blood, it can be more likely to disrupt an endosomal membrane and penetrate a cell. Accordingly, some polymers described herein can selectively penetrate cells and/or deliver nucleic acids at a pH above or below the transition point. Advantageously, some polymers described herein can include an amount of the recurring unit of Formula (XIII) that makes the polymer capable of selectively targeting tumor tissue.

In some embodiments, the polymer can include the following structure, wherein aa and bb are each independently a positive integer in the range of from about 1 to about 2000, and cc is zero or a positive integer in the range of from about 1 to about 2000. In the following structure, a first recurring unit of Formula (I) can be connected to a second recurring unit of Formula (II), a third recurring unit of Formula (III) and/or another first recurring unit of Formula (I). Also, a second recurring unit of Formula (II) can be connected to a first recurring unit of Formula (I), a third recurring unit of Formula (III) and/or another second recurring unit of Formula (II); and a third recurring unit of Formula (III) can be connected to a first recurring unit of Formula (I), a second recurring unit of Formula (II) and/or another third recurring unit of Formula (III).

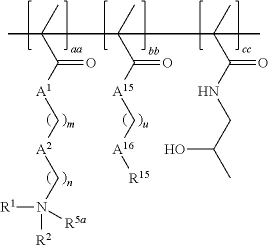

In some embodiments, the polymer can include the following structure, wherein ll, mm, and nn are each independently a positive integer in the range of from about 1 to about 2000, and oo is zero or a positive integer in the range of from about 1 to about 2000. In the following structure, a first recurring unit of Formula (I) can be connected to a second recurring unit of Formula (II), a third recurring unit of Formula (III), a fourth recurring unit of Formula (XIII) and/or another first recurring unit of Formula (I). Similarly, a second recurring unit of Formula (II) can be connected to a first recurring unit of Formula (I), a third recurring unit of Formula (III), a fourth recurring unit of Formula (XIII) and/or another second recurring unit of Formula (II); a third recurring unit of Formula (III) can be connected to a first recurring unit of Formula (I), a second recurring unit of Formula (II), a fourth recurring unit of Formula (XIII) and/or another third recurring unit of Formula (III); and a fourth recurring unit of Formula (XIII) can be connected to a first recurring unit of Formula (I), a second recurring unit of Formula (II), a third recurring unit of Formula (III) and/or another fourth recurring unit of Formula (XIII).

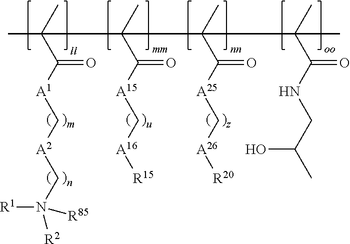

In some embodiments, the polymers described herein may further include a nucleic acid that is associated with the polymer. Nucleic acids can be commercially available and/or designed according to methods known to those skilled in the art. Association of the nucleic acid with the polymer may be carried out, for example, in an aqueous solution or on a solid support, according to methods known to those of ordinary skill in the art as guided by the teachings provided herein.

As described above, a nucleic acid can be covalently bonded to the polymer, e.g., through a linking group. In some embodiments, a polymer that includes a recurring unit of Formula (I) can be intermixed with a moiety that forms the linking group. In some embodiments, the linking group can be covalently bonded with the NR¹R²R^{5a} group of Formula (I). In some embodiments, the moiety that forms the linking group can include PEG. In one embodiment, the moiety that forms the linking group can be PEG covalently bonded to a thiol-reactive agent, including but not limited to iodoacetamide, maleimide, benzylic halide, bromomethylketone, and orthopyridyldisulfide (e.g., PDS) agents. In some embodiments, the thiol-reactive reagent can be covalently bonded to the NR¹R²R^{5a} group of Formula (I) through a PEG linking group.

In some embodiments, a nucleic acid can further be intermixed with the polymer. In some embodiments, the nucleic acid can be modified to include a thiol moiety according to methods known to those skilled in the art. In some embodiments, the nucleic acid can be covalently bonded to the recurring unit of Formula (I) and the linking group through the thiol-reactive agent (e.g., a maleimide agent). In other embodiments, the nucleic acid can replace the thiol-reactive agent (e.g., an iodoacetamide, benzylic halide, bromomethylketone, or orthopyridyldisulfide agent) to be covalently bonded to the linking group.

The nucleic acid may be releasable from the polymer. Advantageously, in some embodiments that include an orthopyridyldisulfide agent, the siRNA may advantageously be released from the polymer under reducing conditions. In some embodiments that include another thiol-reactive agent (e.g., an iodoacetamide, maleimide, benzylic halide, or bromomethylketone), the siRNA may not be released from the polymer under reducing conditions, but may be released into its surroundings as the polymer degrades.

One or more of the recurring units (e.g., a first recurring unit of Formula (I), a second recurring unit of Formula (II), a third recurring unit of Formula (III), a fourth recurring unit of Formula (A), and/or a fifth recurring unit of Formula (XIII)) and/or nucleic acid can be oriented at various positions relative to the polymer. Such positions may be fixed (e.g., at the middle, ends, or side chains of the polymer) or relative, e.g., the polymer may exhibit a configuration in a particular medium (such as an aqueous medium) such that it has interior and exterior portions.

In some embodiments, one or more recurring units of Formula (I) may be oriented at or near the interior of the polymer. In other embodiments, one or more recurring units of Formula (I) may be oriented at or near the exterior of the polymer. In yet other embodiments, one or more recurring units of Formula (II) may be oriented at or near the exterior of the polymer. In still other embodiments, one or more recurring units of Formula (II) may be oriented at or near the interior of the polymer. In some embodiments, one or more recurring units of Formula (III) may be oriented at or near the interior of the polymer. In other embodiments, one or more recurring units of Formula (III) may be oriented at or near the exterior of the polymer. In some embodiments, one or more recurring units of Formula (A) may be oriented at or near the interior of the polymer. In other embodiments, one or more recurring units of Formula (A) may be oriented at or near the exterior of the polymer. In some embodiments, one or more recurring units of Formula (XIII) may be oriented at or near the interior of the polymer. In other embodiments, one or more recurring units of Formula (XIII) may be oriented at or near the exterior of the polymer.

In some embodiments, one or more recurring units of Formula (I) can be oriented at or near the interior of the polymer and one or more recurring units of Formulae (II), (III), (A), and/or (XIII) can be oriented at or near the exterior of the polymer. In some embodiments, substantially all of the recurring units of Formula (I) can be oriented at or near the interior of the polymer and substantially all of the recurring units of Formulae (II), (III), (A), and/or (XIII) can be oriented at or near the exterior of the polymer. In some embodiments, about 50% to about 98%, about 60% to about 95%, about 70% to about 95%, about 80% to about 90%, >about 80% or >about 90% of the recurring units of Formula (I) can be oriented at or near the interior of the polymer. In some embodiments, about 50% to about 98%, about 60% to about 95%, about 70% to about 95%, about 80% to about 90%, >about 80% or >about 90% of the recurring units of Formula (II) can be oriented at or near the exterior of the polymer. In some embodiments, about 50% to about 98%, about 60% to about 95%, about 70% to about 95%, about 80% to about 90%, >about 80% or >about 90% of the recurring units of Formula (III) can be oriented at or near the exterior of the polymer. In some embodiments, about 50% to about 98%, about 60% to about 95%, about 70% to about 95%, about 80% to about 90%, >about 80% or >about 90% of the recurring units of Formula (A) can be oriented at or near the exterior of the polymer. In some embodiments, about 50% to about 98%, about 60% to about 95%, about 70% to about 95%, about 80% to about 90%, >about 80% or >about 90% of the recurring units of Formula (XIII) can be oriented at or near the exterior of the polymer.

In some embodiments, the nucleic acid may be associated with a side chain moiety of the polymer. In other embodiments, the nucleic acid may be associated with an end or terminal recurring unit of the polymer. In yet other embodiments, the nucleic acid may be associated with the middle of the polymer. In still yet other embodiments, the nucleic acid may be associated with the backbone of the polymer. In some embodiments, the nucleic acid may be associated with an exterior moiety or moieties or an exterior surface of the polymer. In other embodiments, the nucleic acid may be associated with an interior moiety or moieties or an interior surface of the polymer. In some embodiments, the nucleic acid can be at least partially contained within the polymer. In other embodiments, the nucleic acid may be substantially completely contained within the polymer. In some embodiments, one or more recurring units of Formula (I) can be oriented at or near the interior of the polymer, one or more recurring units of Formulae (II), (III), (A), and/or (XIII) can be oriented at or near the exterior of the polymer, and the nucleic acid can be at least partially contained within the polymer. In other embodiments, substantially all of the recurring units of Formula (I) can be oriented at or near the interior of the polymer, substantially all of the recurring units of Formulae (II), (III), (A), and/or (XIII) can be oriented at or near the exterior of the polymer, and the nucleic acid can be at least partially contained within the polymer. In some embodiments, about 50% to about 98%, about 60% to about 95%, about 70% to about 95%, about 80% to about 90%, >about 80% or >about 90% of the recurring units of Formula (I) can be oriented at or near the interior of the polymer, and the nucleic acid can be at least partially contained within the polymer. In some embodiments, about 50% to about 98%, about 60% to about 95%, about 70% to about 95%, about 80% to about 90%, >about 80% or >about 90% of the recurring units of Formula (II) can be oriented at or near the exterior of the polymer, and the nucleic acid can be at least partially contained within the polymer. In some embodiments, about 50% to about 98%, about 60% to about 95%, about 70% to about 95%, about 80% to about 90%, >about 80% or >about 90% of the recurring units of Formula (III) can be oriented at or near the exterior of the polymer, and the nucleic acid can be at least partially contained within the polymer. In some embodiments, about 50% to about 98%, about 60% to about 95%, about 70% to about 95%, about 80% to about 90%, >about 80% or >about 90% of the recurring units of Formula (A) can be oriented at or near the exterior of the polymer, and the nucleic acid can be at least partially contained within the polymer. In some embodiments, about 50% to about 98%, about 60% to about 95%, about 70% to about 95%, about 80% to about 90%, >about 80% or >about 90% of the recurring units of Formula (XIII) can be oriented at or near the exterior of the polymer, and the nucleic acid can be at least partially contained within the polymer.

Advantageously, in some embodiments, the recurring unit(s) of Formula (II) can help the polymer disrupt the endosomal membrane to penetrate the cell wall, while the recurring unit(s) of Formula (I) can associate with the nucleic acid and/or shield it from the endosomal membrane. Advantageously, in some embodiments, the recurring unit(s) of Formula (XIII) can cause the polymer to become relatively insoluble and/or hydrophobic at a location having a pH above and/or below the transition point, such as at or near a tumor. Those skilled in the art will recognize that the location and orientation of association may otherwise vary depending on the properties of the specific recurring units and nucleic acid.

Those skilled in the art will recognize that the location and orientation of association may otherwise vary depending on the properties of the specific recurring units and nucleic acid.

Polymers as disclosed herein can be prepared in various ways. In some embodiments, a first reactant can be polymerized to form a polymer that includes at least 95 mole % of recurring units of Formula (I), based on the ratio of moles of recurring units of Formula (I) to the total moles of recurring units in the polymer. In some embodiments, the polymer can be a poly(N-(3-aminopropyl)methacrylamide) homopolymer or a salt thereof. The polymer can be intermixed with a second reactant that includes R¹⁵. In some embodiments, the first reactant can be a salt, such as N-(3-aminopropyl)methacrylamide hydrochloride. In some embodiments, the second reactant can be an optionally substituted C₄-C₂₄ fatty acid (e.g., oleic acid, lauric acid, myristoleic acid, palmitoleic acid, margaric acid, stearic acid, arachidic acid, behenic acid, or lignoceric acid) or an optionally substituted sterol. In some embodiments, the second reactant can be oleic acid. In another embodiment, the second reactant can be butoxymethyl acrylamide. In other embodiments, the second reactant can be hexyl methacrylate. In some embodiments, the polymer can be intermixed with about 0.01 to about 0.7 equivalents of the second reactant, so that about 1% to about 70% of the recurring units of the resulting polymer can be the second recurring unit of Formula (II).

The polymer can be intermixed with one or more additional reactants. In some embodiments, the polymer can be intermixed with an additional reactant (e.g., a third or fourth reactant) that can form a recurring unit of Formula (A). In some embodiments the additional reactant that can form a recurring unit of Formula (A) can be a poly(ethylene glycol) methacrylate. In other embodiments the additional reactant that can form a recurring unit of Formula (A) can be a methoxy polyethylene glycol methacrylate. In some embodiments the additional reactant that can form a recurring unit of Formula (A) can be ethoxyethyl methacrylate. In some embodiments, the polymer can be intermixed with about 1 to about 6 equivalents of the additional reactant that can form a recurring unit of Formula (A). In some embodiments, the polymer can be intermixed with about 0.01 to about 0.7 equivalents of the additional reactant, so that about 1% to about 70% of the recurring units of the resulting polymer can be a recurring unit of Formula (A).

In some embodiments, the polymer that includes a recurring unit of Formula (I) and a recurring unit of Formula (II), or that includes a recurring unit of Formula (I), a recurring unit of Formula (II), and a recurring unit of Formula (A), can be intermixed with a reactant (e.g., a third or fourth reactant) that includes R²⁰. In some embodiments, the reactant can include a pH transition group and a group that is reactive with the first reactant. For example, the reactant can include a succinate moiety. In another example, the reactant can be succinic acid or succinic anhydride. In yet another example, the reactant can include an amine moiety, such as those described herein. In some embodiments, the reactant can be morpholine moiety, for example, 4-(2-isothiocyanatoethyl)morpholine. In some embodiments, the polymer can be intermixed with about 1 to about 6 equivalents of the reactant that includes R²⁰. In some embodiments, the polymer can be intermixed with about 0.01 to about 0.7 equivalents of the additional reactant, so that about 1% to about 70% of the recurring units of the resulting polymer can be a recurring unit of Formula (XIII).

Those skilled in the art may appreciate that in some embodiments, the polymer that includes at least 95 mole % of recurring units of Formula (I) can be intermixed with a second reactant that includes R¹⁵ and a reactant that can form a recurring unit of Formula (A). In other embodiments, the polymer that includes at least 95 mole % of recurring units of Formula (I) can be intermixed with a second reactant that includes R¹⁵ and a reactant that includes R²⁰. In other embodiments, the polymer that includes at least 95 mole % of recurring units of Formula (I) can be intermixed with a second reactant that includes R¹⁵, a reactant that can form a recurring unit of Formula (A), and a reactant that includes R²⁰. Those skilled in the art may also appreciate that the reactants can be intermixed with the polymer in any order.

In other embodiments, a monomeric first reactant that forms a recurring unit of Formula (I) and a monomeric second reactant that forms a recurring unit of Formula (II) can be intermixed to form a polymer. In some embodiments, the first reactant can be N-(3-aminopropyl)methacrylamide hydrochloride, which can be intermixed with the second reactant, which can be a monomer that includes R¹⁵. In some embodiments, about 1 to about 6 equivalents of the second monomeric reactant can be intermixed with the first monomeric reactant.

One or more additional monomeric reactants can be intermixed with the reaction mixture. In some embodiments, an additional (e.g., third or fourth) monomeric reactant that forms a recurring unit of Formula (A) can be intermixed with the first and/or second monomeric reactants. The additional monomeric reactant can be methyl polyethylene glycol methacrylate (mPEGMA). In some embodiments, about 1 to about 20 equivalents of the additional monomeric reactant that forms a recurring unit of Formula (A) can be intermixed with the first and/or second monomeric reactants.

In other embodiments, the reaction mixture can be intermixed with an additional monomeric reactant that includes R²⁰. In some embodiments, the additional monomeric reactant can be a methacrylate monomer that includes a group having a pH transition point. For example, the additional monomeric reactant can include a morpholine moiety. In some embodiments, the additional monomeric reactant can be morpholinoethyl methacrylate. The monomers can be polymerized using methods known to those skilled in the art, such as by free radical polymerization. In some embodiments, the monomers can be polymerized via reversible addition-fragmentation chain transfer (RAFT) polymerization. In some embodiments, about 1 to about 6 equivalents of the additional monomeric reactant that includes R²⁰ can be intermixed with the first and/or second monomeric reactants.

Those skilled in the art may appreciate that in some embodiments, a first monomeric reactant that forms a recurring unit of Formula (I) can be intermixed with a second monomeric reactant that includes R¹⁵ and a third monomeric reactant that can form a recurring unit of Formula (A). In other embodiments, a first monomeric reactant that forms a recurring unit of Formula (I) can be intermixed with a second monomeric reactant that includes R¹⁵ and a third monomeric reactant that includes R²⁰. In yet other embodiments, a first monomeric reactant that forms a recurring unit of Formula (I) can be intermixed with a second monomeric reactant that includes R¹⁵, a third monomeric reactant that can form a recurring unit of Formula (A), and a fourth recurring unit that includes R²⁰. In some embodiments, the first, second, third, and/or fourth reactants are reacted together via a coupling reaction. Those skilled in the art may also appreciate that the first, second, third, and/or fourth monomeric reactants can be intermixed in any order.

Polymers that can include a first recurring unit of Formula (I), a second recurring unit of Formula (II), and a third recurring unit of Formula (III) as disclosed herein can be prepared in various ways. For example, a first reactant, a second reactant, and a third reactant can be intermixed to form a polymer. In some embodiments, a first monomeric reactant can be intermixed with a second monomeric reactant to form a copolymer. In some embodiments, the first monomeric reactant can form a recurring unit of Formula (I) and the second monomeric reactant can faun a recurring unit of Formula (III). In some embodiments, at least 95 mole % of the recurring units in the copolymer are recurring units of Formula (I) and (III), based on the ratio of the moles of recurring units of Formulae (I) and (III) to the total moles of recurring units in the copolymer. In some embodiments, the first monomeric reactant can be a salt, such as N-(3-aminopropyl)methacrylamide hydrochloride. In some embodiments, the second reactant can be N-(2-hydroxypropyl)methacrylamide (HPMA). The resulting copolymer can be intermixed with a third reactant that that includes R¹⁵. In some embodiments, the third reactant can be an optionally substituted C₄-C₂₄ fatty acid (e.g., oleic acid, lauric acid, myristoleic acid, palmitoleic acid, margaric acid, stearic acid, arachidic acid, behenic acid, or lignoceric acid) or an optionally substituted sterol. In some embodiments, the third reactant can be oleic acid. In other embodiments, the third reactant can be butoxymethyl acrylamide. In another embodiment, the third reactant can be hexyl methacrylate. In some embodiments, the polymer can be intermixed with about 0.01 to about 0.7 equivalents of the third reactant so that about 1% to about 70% of the recurring units of the resulting polymer can be recurring units of Formula (II).

The copolymer that includes a recurring unit of Formula (I), a recurring unit of Formula (II) and a recurring unit of Formula (III) can be intermixed with one or more additional reactants. In some embodiments, the copolymer can be intermixed with an additional reactant (e.g., a fourth or fifth reactant) that can form a recurring unit of Formula (A). In some embodiments the additional reactant that can form a recurring unit of Formula (A) can be a poly(ethylene glycol) methacrylate. In other embodiments the additional reactant that can form a recurring unit of Formula (A) can be a methoxy polyethylene glycol methacrylate. In some embodiments the additional reactant that can form a recurring unit of Formula (A) can be ethoxyethyl methacrylate. In some embodiments, the copolymer can be intermixed with about 1 to about 6 equivalents of the additional reactant. In some embodiments, the copolymer can be intermixed with about 0.01 to about 0.7 equivalents of the additional reactant(s), so that about 1% to about 70% of the recurring units of the resulting copolymer can be a recurring unit of Formula (A).

In some embodiments, the copolymer that includes a recurring unit of Formula (I), a recurring unit of Formula (II) and a recurring unit of Formula (III) or that includes a recurring unit of Formula (I), a recurring unit of Formula (II), a recurring unit of Formula (III) and a recurring unit of Formula (A) can be intermixed with a reactant (e.g., a fourth or fifth reactant) that includes R²⁰. In some embodiments, the reactant can include a pH transition group and a group that is reactive with the first reactant. For example, the reactant can include a succinate moiety. In another example, the reactant can be succinic acid or succinic anhydride. In yet another example, the reactant can include an amine moiety, such as those described herein. In some embodiments, the reactant can be morpholine moiety, for example, 4-(2-isothiocyanatoethyl)morpholine. In some embodiments, the copolymer can be intermixed with about 1 to about 6 equivalents of the additional reactant(s). In some embodiments, the copolymer can be intermixed with about 0.01 to about 0.7 equivalents of the additional reactant, so that about 1% to about 70% of the recurring units of the resulting copolymer can be a recurring unit of Formula (XIII).

Those skilled in the art may appreciate that in some embodiments, the copolymer that includes at least 95 mole % of recurring units of Formulae (I) and (III) can be intermixed with a third reactant that includes R¹⁵ and a fourth reactant that can form a recurring unit of Formula (A). In other embodiments, the copolymer that includes at least 95 mole % of recurring units of Formulae (I) and (III) can be intermixed with a third reactant that includes R¹⁵ and a fourth reactant that includes R²⁰. In other embodiments, the copolymer that includes at least 95 mole % of recurring units of Formulae (I) and (III) can be intermixed with a third reactant that includes R¹⁵, a fourth reactant that can form a recurring unit of Formula (A), and a fifth reactant that includes R²⁰. Those skilled in the art may also appreciate that the third, fourth, and/or fifth reactants can be intermixed with the copolymer in any order.

In other embodiments, the first reactant can be a monomer that forms a recurring unit of Formula (I), the second reactant can be a monomer that forms a recurring unit of Formula (II), and the third reactant can be a monomer that forms a recurring unit of Formula (III). In some embodiments, the first reactant can be an optionally substituted aminoalkyl methacrylamide or an optionally substituted aminoalkyl methacrylate, such as N-(3-aminopropyl)methacrylamide, 2-aminoethyl methacrylate, or dimethylamino ethyl methacrylate (DMAEMA). In some embodiments, the first reactant can be cystamine methacrylamide. In other embodiments, the second reactant can be an optionally substituted C₄-C₂₄ alkyl methacrylate, an optionally substituted C₄-C₂₄ alkenyl methacrylate (e.g., oleic methacrylate, lauric methacrylate, myristoleic methacrylate, palmitoleic methacrylate, margaric methacrylate, stearic methacrylate, arachidic methacrylate, behenic methacrylate, or lignoceric methacrylate), or an optionally substituted sterol. In yet other embodiments, the third reactant can be N-(2-hydroxypropyl)methacrylamide (HPMA). The monomers can be polymerized using methods known to those skilled in the art, such as by free radical polymerization. In some embodiments, the monomers can be polymerized via reversible addition-fragmentation chain transfer (RAFT) polymerization. In some embodiments, a monomeric first reactant can be intermixed with about 1 to about 6 equivalents of a second monomeric reactant and/or about 5 to about 20 equivalents of a third monomeric reactant.

One or more additional (e.g., fourth or fifth) monomeric reactants can be intermixed with the reaction mixture. In some embodiments, an additional monomeric reactant that forms a recurring unit of Formula (A) can be intermixed with the first, second, and/or third monomeric reactants. The additional monomeric reactant can be methyl polyethylene glycol methacrylate (mPEGMA). In some embodiments, about 1 to about 20 equivalents of the additional monomeric reactant can be intermixed with the first, second, and/or third monomeric reactants.

In other embodiments, the reaction mixture can be intermixed with an additional monomeric reactant that includes R²⁰. In some embodiments, the additional monomeric reactant can be a methacrylate monomer that includes a group having a pH transition point. For example, the additional monomeric reactant can include a morpholine moiety. In some embodiments, the additional monomeric reactant can be morpholinoethyl methacrylate. The monomers can be polymerized using methods known to those skilled in the art, such as by free radical polymerization. In some embodiments, the monomers can be polymerized via reversible addition-fragmentation chain transfer (RAFT) polymerization. In some embodiments, about 1 to about 6 equivalents of the additional monomeric reactant that includes R²⁰ can be intermixed with the first, second, and/or third monomeric reactants.

Those skilled in the art may appreciate that in some embodiments, a first monomeric reactant that forms a recurring unit of Formula (I) can be intermixed with a second monomeric reactant that includes R¹⁵, a third monomeric reactant that can form a recurring unit of Formula (III), and a fourth recurring unit that can form a recurring unit of Formula (A). In other embodiments, a first monomeric reactant that forms a recurring unit of Formula (I) can be intermixed with a second monomeric reactant that includes R¹⁵, a third monomeric reactant that can form a recurring unit of Formula (III), and a fourth recurring unit that includes R²⁰. in some embodiments, a first monomeric reactant that forms a recurring unit of Formula (I) can be intermixed with a second monomeric reactant that includes R¹⁵, a third monomeric reactant that can form a recurring unit of Formula (III), a fourth recurring unit that can form a recurring unit of Formula (A), and a fifth recurring unit that includes R²⁰. In some embodiments, the first, second, third, fourth, and/or fifth reactants are reacted together via a coupling reaction. Those skilled in the art may also appreciate that the first, second, third, fourth, and/or fifth monomeric reactants can be intermixed in any order.

The various reactants can be intermixed in various manners. Two or more reactants can be intermixed at substantially the same time, and/or one or more reactants can be intermixed sequentially. In some embodiments, the first reactant can be intermixed with the second reactant, the third reactant, the fourth reactant and the fifth reactant at about the same time. In other embodiments, the first reactant, the second reactant, third reactant, fourth reactant and/or fifth reactant can be intermixed sequentially.

In some embodiments, the reactants can be intermixed with one or more solvents, such as an organic solvent. Examples of organic solvents include, but are not limited to, dimethylformamide (DMF) and dimethyl sulfoxide (DMSO). The reactants and/or solvents may be commercially available and/or may be synthesized according to methods known to those of ordinary skill in the art as guided by the teachings provided herein.

In some embodiments, the reactants can be intermixed in the presence of a suitable base. Suitable bases are known to those skilled in the art. Examples of bases include, but are not limited to, an amine base, such as an alkylamine (including mono-, di- and tri-alkylamines (e.g., triethylamine)).

In some embodiments, the reactants can be intermixed in the presence of a coupling agent. Any suitable coupling agent may be used. In some embodiments, the coupling agent can be selected from 1-ethyl-3-(3-dimethylaminopropyl)-carbodiimide (EDC), 1,3-dicyclohexyl carbodiimide (DCC), 1,1′-carbonyl-diimidazole (CDI), N,N′-disuccinimidyl carbonate (DSC), N-[(dimethylamino)-1H-1,2,3-triazolo-[4,5-b]pyridine-1-yl-methylene]-N-methylmethanaminium hexafluorophosphate N-oxide (HATU), 2-[(1H-benzotriazol-1-yl)-1,1,3,3-tetramethylaminium hexafluorophosphate (HBTU), 2-[(6-chloro-1H-benzotriazol-1-yl)-1,1,3,3-tetramethylaminium hexafluorophosphate (HCTU), benzotriazole-1-yl-oxy-tris-pyrrolidino-phosphonium hexafluorophosphate (PyBOP®), bromo-tris-pyrrolidino-phosphonium hexafluorophosphate (PyBroP®), 2-[(1H-benzotriazol-1-yl)-1,1,3,3-tetramethylaminium tetrafluoroborate (TBTU), and benzotriazol-1-yl-oxy-tris-(dimethylamino)phosphonium hexafluorophosphate (BOP).

In some embodiments, the reaction may be carried out at room temperature. In some embodiments, the reaction mixture may be stirred for several hours. The reaction products may be isolated by any means known in the art including chromatographic techniques. In some embodiments, the solvent may be evaporated to recover the reaction product (e.g., via rotary evaporation). In other embodiments, the reaction product may be removed by precipitation followed by centrifugation.

A wide variety of polymers comprising the recurring units described herein may be made by varying the molecular weights and structures of the reactants, the size and type of the R groups on the reactants, and/or the mole ratios of the first reactant to second, third, fourth, and/or fifth reactant. In addition, mixtures of different first reactants and/or mixtures of different second reactants and/or mixtures of different third reactants and/or mixtures of different fourth reactants and/or mixtures of different fifth reactants may be used. Furthermore, additional reactants of varying sizes, molecular weights, and/or structures may advantageously be used to incorporate additional recurring units into the polymer.

Some embodiments herein are directed to a block copolymer that can include a first subunit that is hydrophilic; and a second subunit that can include various other recurring units. In some embodiments, the first subunit can include a recurring unit selected from Formula (E) and Formula (F) having the following structures:

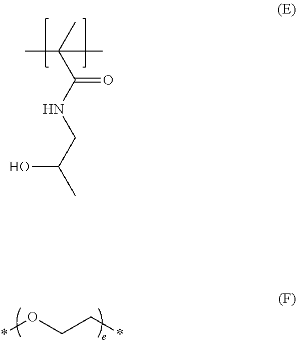

In some embodiments, the first subunit can include pHPMA or a salt thereof. In some embodiments, the first subunit can include at least 75 mole % of recurring units of Formula (E) based on the ratio of total moles of recurring units of Formula (E) to the total moles of recurring units in the first subunit. In some embodiments, the first subunit can include at least 85 mole % of recurring units of Formula (E) based on the ratio of total moles of recurring units of Formula (E) to the total moles of recurring units in the first subunit. In some embodiments, the first subunit can include at least 95 mole % of recurring units of Formula (E) based on the ratio of total moles of recurring units of Formula (E) to the total moles of recurring units in the first subunit. In some embodiments, the first subunit can include at least 98 mole % of recurring units of Formula (E) based on the ratio of total moles of recurring units of Formula (E) to the total moles of recurring units in the first subunit. In some embodiments, the first subunit can include at least 99 mole % of recurring units of Formula (E) based on the ratio of total moles of recurring units of Formula (E) to the total moles of recurring units in the first subunit. In some embodiments, the first subunit can include about 100 mole % of recurring units of Formula (E) based on the ratio of total moles of recurring units of Formula (E) to the total moles of recurring units in the first subunit. In some embodiments, the first subunit can be a homopolymer of recurring units of Formula (E). For example, the first subunit can be a homopolymer of pHPMA or a salt thereof.

In some embodiments, the first subunit can include a total amount of the recurring units of Formula (E) in the range of about 50 mole % to about 99 mole % based on the ratio of total moles of recurring units of Formula (E) to the total moles of recurring units in the first subunit. In some embodiments, the first subunit can include a total amount of the recurring units of Formula (E) in the range of about 70 mole % to about 98 mole % based on the ratio of total moles of recurring units of Formula (E) to the total moles of recurring units in the first subunit. In some embodiments, the first subunit can include a total amount of the recurring units of Formula (E) in the range of about 80 mole % to about 95 mole % based on the ratio of total moles of recurring units of Formula (E) to the total moles of recurring units in the subunit. In some embodiments, the first subunit can include a total amount of the recurring units of Formula (E) in the range of about 90 mole % to about 99 mole % based on the ratio of total moles of recurring units of Formula (E) to the total moles of recurring units in the first subunit. In some embodiments, the first subunit can include a total amount of the recurring units of Formula (E) of about 50 mole %, about 60 mole %, about 70 mole %, about 80 mole %, about 90 mole %, about 95 mole %, about 98 mole %, about 99 mole % or about 100 mole % based on the ratio of total moles of recurring units of Formula (E) to the total moles of recurring units in the first subunit.

In some embodiments, the first subunit can include at least about 50 recurring units of Formula (E). In other embodiments, the first subunit can include at least about 100 recurring units of Formula (E). In other embodiments, the first subunit can include at least about 200 recurring units of Formula (E). In other embodiments, the first subunit can include at least about 500 recurring units of Formula (E). In other embodiments, the first subunit can include at least about 1000 recurring units of Formula (E). In other embodiments, the first subunit can include at least about 1500 recurring units of Formula (E). In other embodiments, the first subunit can include about 2000 recurring units of Formula (E).

In some embodiments, the first subunit can include from about 50 to about 2000 recurring units of Formula (E). In other embodiments, the first subunit can include 200 to about 1500 recurring units of Formula (E). In yet other embodiments, the first subunit can include about 300 to about 700 recurring units of Formula (E). In other embodiments, the first subunit can include from about 50 to about 100 recurring units, from about 1500 to about 2000 recurring units, from about 1000 to about 1500 recurring units, or from about 700 to about 1000 recurring units of Formula (E). In yet other embodiments, the first subunit can include from about 100 to about 200, from about 100 to about 500, or from about 300 to about 600 recurring units of Formula (E).

In Formula (F), e can be an integer in the range of 1 to 300. In some embodiments, e can be an integer in the range of 1 to 250. In other embodiments, e can be an integer in the range of 1 to 100. In yet other embodiments, e can be an integer in the range of 1 to 50. In still yet other embodiments, e can be an integer in the range of 1 to 25. In some embodiments, e can be an integer in the range of 1 to 15. In other embodiments, e can be an integer in the range of 25 to 250. In yet other embodiments, e can be an integer in the range of 25 to 75. In still other embodiments, e can be an integer in the range of 75 to 125. In other embodiments, e can be an integer in the range of 125 to 175. In even other embodiments, e can be an integer in the range of 175 to 225.

The molecular weight of the recurring unit of Formula (F) can vary. In some embodiments, the recurring unit of Formula (F) can have a molecular weight in the range of from about 1 kDa to about 15 kDa. In other embodiments, the molecular weight of the recurring unit of Formula (F) can be in the range of from about 2 kDa to about 10 kDa. In other embodiments, the molecular weight of the recurring unit of Formula (F) can be in the range of from about 1 kDa to about 3 kDa. In other embodiments, the molecular weight of the recurring unit of Formula (F) can be in the range of from about 4 kDa to about 6 kDa. In other embodiments, the molecular weight of the recurring unit of Formula (F) can be in the range of from about 9 kDa to about 11 kDa. In some embodiments, the molecular weight of the recurring unit of Formula (F) can be about 2 kDa. In other embodiments, the molecular weight of the recurring unit of Formula (F) can be about 5 kDa. In still other embodiments, the molecular weight of the recurring unit of Formula (F) can be about 10 kDa.

In some embodiments, the recurring unit of Formula (F) can be included in a recurring unit of Formula (Fa):

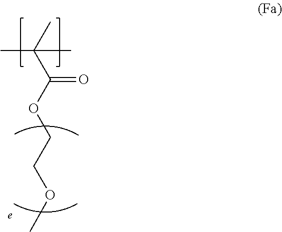

In some embodiments, the first subunit can include poly(polyethylene glycol methacrylate (pPEGMA). In other embodiments, the recurring unit of Formula (F) can be incorporated into a terminal unit. In some embodiments, the terminal unit can include methyl polyethylene glycol (mPEG). In some embodiments, the recurring unit of Formula (F) can be included in a terminal unit of Formula (Fb):

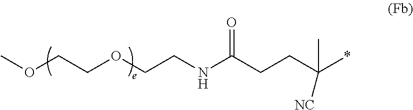

In some embodiments, the first subunit can include at least 75 mole % of recurring units of Formula (F) based on the ratio of total moles of recurring units of Formula (F) to the total moles of recurring units in the first subunit. In some embodiments, the first subunit can include at least 85 mole % of recurring units of Formula (F) based on the ratio of total moles of recurring units of Formula (F) to the total moles of recurring units in the first subunit. In some embodiments, the first subunit can include at least 95 mole % of recurring units of Formula (F) based on the ratio of total moles of recurring units of Formula (F) to the total moles of recurring units in the first subunit. In some embodiments, the first subunit can include at least 98 mole % of recurring units of Formula (F) based on the ratio of total moles of recurring units of Formula (F) to the total moles of recurring units in the first subunit. In some embodiments, the first subunit can include at least 99 mole % of recurring units of Formula (F) based on the ratio of total moles of recurring units of Formula (F) to the total moles of recurring units in the first subunit. In some embodiments, the first subunit can include about 100 mole % of recurring units of Formula (F) based on the ratio of total moles of recurring units of Formula (F) to the total moles of recurring units in the first subunit. In some embodiments, the first subunit can be a homopolymer of recurring units of Formula (F). For example, the first subunit can be a homopolymer of pPEGMA.

In some embodiments, the first subunit can include a total amount of the recurring units of Formula (F) in the range of about 50 mole % to about 99 mole % based on the ratio of total moles of recurring units of Formula (F) to the total moles of recurring units in the first subunit. In some embodiments, the first subunit can include a total amount of the recurring units of Formula (F) in the range of about 70 mole % to about 98 mole % based on the ratio of total moles of recurring units of Formula (F) to the total moles of recurring units in the first subunit. In some embodiments, the first subunit can include a total amount of the recurring units of Formula (F) in the range of about 80 mole % to about 95 mole % based on the ratio of total moles of recurring units of Formula (F) to the total moles of recurring units in the subunit. In some embodiments, the first subunit can include a total amount of the recurring units of Formula (F) in the range of about 90 mole % to about 99 mole % based on the ratio of total moles of recurring units of Formula (F) to the total moles of recurring units in the first subunit. In some embodiments, the first subunit can include a total amount of the recurring units of Formula (F) of about 50 mole %, about 60 mole %, about 70 mole %, about 80 mole %, about 90 mole %, about 95 mole %, about 98 mole %, about 99 mole % or about 100 mole % based on the ratio of total moles of recurring units of Formula (F) to the total moles of recurring units in the first subunit.

In some embodiments, the first subunit can include at least about 50 recurring units of Formula (F). In other embodiments, the first subunit can include at least about 100 recurring units of Formula (F). In other embodiments, the first subunit can include at least about 200 recurring units of Formula (F). In other embodiments, the first subunit can include at least about 500 recurring units of Formula (F). In other embodiments, the first subunit can include at least about 1000 recurring units of Formula (F). In other embodiments, the first subunit can include at least about 1500 recurring units of Formula (F). In other embodiments, the first subunit can include about 2000 recurring units of Formula (F).

In some embodiments, the first subunit can include from about 50 to about 2000 recurring units of Formula (F). In other embodiments, the first subunit can include 200 to about 1500 recurring units of Formula (F). In yet other embodiments, the first subunit can include about 300 to about 700 recurring units of Formula (F). In other embodiments, the first subunit can include from about 50 to about 100 recurring units, from about 1500 to about 2000 recurring units, from about 1000 to about 1500 recurring units, or from about 700 to about 1000 recurring units of Formula (F). In yet other embodiments, the first subunit can include from about 100 to about 200, from about 100 to about 500, or from about 300 to about 600 recurring units of Formula (F).

As stated previously, the second subunit can include various other recurring units. In some embodiments, the second subunit can include a recurring unit of Formula (I) and a recurring unit of Formula (II) as described herein. In some embodiments, the second subunit can include a recurring unit of Formula (I), a recurring unit of Formula (II), and a recurring unit of Formula (III) as described herein. In some embodiments, the second subunit can include a recurring unit of Formula (I), a recurring unit of Formula (II), and a recurring unit of Formula (A) as described herein. In some embodiments, the second subunit can include a recurring unit of Formula (I), a recurring unit of Formula (II), and a recurring unit of Formula (XIII) as described herein. In some embodiments, the second subunit can include a recurring unit of Formula (I), a recurring unit of Formula (II), a recurring unit of Formula (III), and a recurring unit of Formula (A) as described herein. In some embodiments, the second subunit can include a recurring unit of Formula (I), a recurring unit of Formula (II), a recurring unit of Formula (III), and a recurring unit of Formula (XIII) as described herein. In some embodiments, the second subunit can include a recurring unit of Formula (I), a recurring unit of Formula (II), a recurring unit of Formula (A), and a recurring unit of Formula (XIII) as described herein. In some embodiments, the second subunit can include a recurring unit of Formula (I), a recurring unit of Formula (II), a recurring unit of Formula (III), a recurring unit of Formula (A), and a recurring unit of Formula (XIII) as described herein. The amounts and ratios of the recurring units present in the second subunit can be the same as those for the polymers described herein.

The relative amounts of the first subunit and the second subunit present in the block copolymer can vary widely. In some embodiments, the block copolymer can include ≧90% mole percent of recurring units of the first subunit based on the ratio of total moles of recurring units in the first subunit to the total moles of recurring units in the block polymer. In some embodiments, the block copolymer can include ≧80% mole percent of recurring units of the first subunit based on the ratio of total moles of recurring units in the first subunit to the total moles of recurring units in the block polymer. In some embodiments, the block copolymer can include ≧50% mole percent of recurring units of the first subunit based on the ratio of total moles of recurring units in the first subunit to the total moles of recurring units in the block polymer. In some embodiments, the block copolymer can include ≧30% mole percent of recurring units of the first subunit based on the ratio of total moles of recurring units of the first subunit to the total moles of recurring units in the block polymer. In some embodiments, the block copolymer can include ≧15% mole percent of recurring units of the first subunit based on the ratio of total moles of recurring units in the first subunit to the total moles of recurring units in the block polymer.

In some embodiments, the block copolymer can include a total amount of the recurring units of the first subunit in the range of about 10 mole % to about 90 mole % based on the ratio of total moles of recurring units in the first subunit to the total moles of recurring units in the block copolymer. In some embodiments, the block copolymer can include a total amount of the recurring units of the first subunit in the range of about 20 mole % to about 60 mole % based on the ratio of total moles of recurring units in the first subunit to the total moles of recurring units in the block copolymer. In some embodiments, the block copolymer can include a total amount of the recurring units of the first subunit in the range of about 60 mole % to about 50 mole % based on the ratio of total moles of recurring units in the first subunit to the total moles of recurring units in the block copolymer. In some embodiments, the block copolymer can include a total amount of recurring units of the first subunit of about 10 mole %, about 20 mole %, about 30 mole %, about 40 mole %, about 50 mole %, about 60 mole %, about 70 mole %, about 80 mole %, or about 90 mole % based on the ratio of total moles of recurring units of the first subunit to the total moles of recurring units in the block copolymer.

In some embodiments, the block copolymer can include at least about 50 recurring units in the first subunit (e.g., at least 50 recurring units of Formulae (E) and/or (F)). In other embodiments, the block copolymer can include at least about 100 recurring units in the first subunit. In other embodiments, the block copolymer can include at least about 200 recurring units in the first subunit. In other embodiments, the block copolymer can include at least about 500 recurring units in the first subunit. In other embodiments, the block copolymer can include at least about 1000 recurring units in the first subunit. In other embodiments, the block copolymer can include at least about 1500 recurring units in the first subunit. In other embodiments, the block copolymer can include about 2000 recurring units in the first subunit.

In some embodiments, the block copolymer can include from about 50 to about 2000 recurring units in the first subunit. In other embodiments, the block copolymer can include 200 to about 1500 recurring units in the first subunit. In yet other embodiments, the block copolymer can include about 300 to about 700 recurring units in the first subunit. In other embodiments, the block copolymer can include from about 50 to about 100 recurring units, from about 1500 to about 2000 recurring units, from about 1000 to about 1500 recurring units, or from about 700 to about 1000 recurring units in the first subunit. In yet other embodiments, the block copolymer can include from about 100 to about 200, from about 100 to about 500, or from about 300 to about 600 recurring units in the first subunit.

In some embodiments, the block copolymer can include ≧90% mole percent of recurring units of the second subunit based on the ratio of total moles of recurring units in the second subunit to the total moles of recurring units in the block polymer. In some embodiments, the block copolymer can include ≧80% mole percent of recurring units of the second subunit based on the ratio of total moles of recurring units in the second subunit to the total moles of recurring units in the block polymer. In some embodiments, the block copolymer can include ≧50% mole percent of recurring units of the second subunit based on the ratio of total moles of recurring units in the second subunit to the total moles of recurring units in the block polymer. In some embodiments, the block copolymer can include ≧30% mole percent of recurring units of the second subunit based on the ratio of total moles of recurring units of the second subunit to the total moles of recurring units in the block polymer. In some embodiments, the block copolymer can include ≧15% mole percent of recurring units of the second subunit based on the ratio of total moles of recurring units in the second subunit to the total moles of recurring units in the block polymer.

In some embodiments, the block copolymer can include a total amount of the recurring units of the second subunit in the range of about 10 mole % to about 90 mole % based on the ratio of total moles of recurring units in the second subunit to the total moles of recurring units in the block copolymer. In some embodiments, the block copolymer can include a total amount of the recurring units of the second subunit in the range of about 20 mole % to about 60 mole % based on the ratio of total moles of recurring units in the second subunit to the total moles of recurring units in the block copolymer. In some embodiments, the block copolymer can include a total amount of the recurring units of the second subunit in the range of about 60 mole % to about 50 mole % based on the ratio of total moles of recurring units in the second subunit to the total moles of recurring units in the block copolymer. In some embodiments, the block copolymer can include a total amount of recurring units of the second subunit of about 10 mole %, about 20 mole %, about 30 mole %, about 40 mole %, about 50 mole %, about 60 mole %, about 70 mole %, about 80 mole %, or about 90 mole % based on the ratio of total moles of recurring units of the second subunit to the total moles of recurring units in the block copolymer.

In some embodiments, the block copolymer can include at least about 50 recurring units in the second subunit (e.g., at least 50 recurring units of Formulae (I), (II), (III), (XIII), and/or (A)). In other embodiments, the block copolymer can include at least about 100 recurring units in the second subunit. In other embodiments, the block copolymer can include at least about 200 recurring units in the second subunit. In other embodiments, the block copolymer can include at least about 500 recurring units in the second subunit. In other embodiments, the block copolymer can include at least about 1000 recurring units in the second subunit. In other embodiments, the block copolymer can include at least about 1500 recurring units in the second subunit. In other embodiments, the block copolymer can include about 2000 recurring units in the second subunit.

In some embodiments, the block copolymer can include from about 50 to about 2000 recurring units in the second subunit. In other embodiments, the block copolymer can include 200 to about 1500 recurring units in the second subunit. In yet other embodiments, the block copolymer can include about 300 to about 700 recurring units in the second subunit. In other embodiments, the block copolymer can include from about 50 to about 100 recurring units, from about 1500 to about 2000 recurring units, from about 1000 to about 1500 recurring units, or from about 700 to about 1000 recurring units in the second subunit. In yet other embodiments, the block copolymer can include from about 100 to about 200, from about 100 to about 500, or from about 300 to about 600 recurring units in the second subunit.

The number of blocks in the block copolymer can vary. In some embodiments, the block copolymer can be a diblock copolymer. In other embodiments, it can be a triblock copolymer. In other embodiments, the block copolymer can include four, five, six, or more blocks. In some embodiments, the block copolymer can be a diblock copolymer that includes a first subunit and a second subunit as described herein.

In some embodiments, a majority of the recurring units in the block copolymer are recurring units that comprise the first or second subunits. In some embodiments, at least 50 mole % of the total moles of recurring units in the block copolymer can be recurring units that comprise the first and second subunits (e.g., the sum of the moles of recurring units of Formula (E), moles of recurring units of Formula (F), moles of recurring units of Formula (I), moles of recurring units of Formula (II), moles of recurring units of Formula (A), and moles of recurring units of Formula (XIII) in the block copolymer can be equal to at least 50 mole % of the total moles of recurring units in the block copolymer). In some embodiments, at least 75 mole % of the total moles of recurring units in the block copolymer can be recurring units that comprise the first and second subunits. In some embodiments, at least 85 mole % of the total moles of recurring units in the block copolymer can be recurring units that comprise the first and second subunits. In some embodiments, at least 95 mole % of the total moles of recurring units in the block copolymer can be recurring units that comprise the first and second subunits. In some embodiments, at least 98 mole % of the total moles of recurring units in the block copolymer can be recurring units that comprise the first and second subunits. In some embodiments, at least 99 mole % of the total moles of recurring units in the block copolymer can be recurring units that comprise the first and second subunits. In some embodiments, about 100 mole % of the total moles of recurring units in the block copolymer can be recurring units that comprise the first and second subunits (e.g., the block copolymer does not include any recurring units other than the recurring units of Formulae (E), (F), (I), (II), (A), and (XIII)).

In some embodiments, the block copolymer can include a total amount of recurring units that comprise the first and second subunits in the range of from about 50 mole % to about 99 mole % based on the ratio of total moles of recurring units that comprise the first and second subunits to the total moles of recurring units in the block copolymer (e.g., the sum of the moles of recurring units of Formula (E), moles of recurring units of Formula (F), moles of recurring units of Formula (I), moles of recurring units of Formula (II), moles of recurring units of Formula (A), and moles of recurring units of Formula (XIII) in the block copolymer can be in the range of from about 50 mole % to about 99 mole % based on the total moles of recurring units in the block copolymer). In some embodiments, the block copolymer can include a total amount of recurring units that comprise the first and second subunits in the range of from about 70 mole % to about 98 mole % based on the ratio of total moles of recurring units that comprise the first and second subunits to the total moles of recurring units in the block copolymer. In some embodiments, the block copolymer can include a total amount of recurring units that comprise the first and second subunits in the range of from about 80 mole % to about 95 mole % based on the ratio of total moles of recurring units that comprise the first and second subunits to the total moles of recurring units in the block copolymer. In some embodiments, the block copolymer can include a total amount of recurring units that comprise the first and second subunits in the range of from about 90 mole % to about 99 mole % based on the ratio of total moles of recurring units that comprise the first and second subunits to the total moles of recurring units in the block copolymer. In some embodiments, the block copolymer can include a total amount of recurring units that comprise the first and second subunits of about 50 mole %, about 60 mole %, about 70 mole %, about 80 mole %, about 90 mole %, about 95 mole %, about 98 mole %, about 99 mole % or about 100 mole % based on the ratio of total moles of recurring units that comprise the first and second subunits to the total moles of recurring units in the block copolymer.

In some embodiments directed to a block copolymer that includes a first subunit and a second subunit, the block copolymer can include from about 10 mole % to about 90 mole % of recurring units that comprise the first subunit, based on the ratio of total moles of recurring units that comprise the first subunit to the total moles of recurring units in the block copolymer; and from about 10 mole % to about 90 mole % of recurring units that comprise the second subunit, based on the ratio of total moles of recurring units that comprise the second subunit to the total moles of recurring units in the block copolymer; and wherein about 95 mole % to about 99 mole % of the total moles of recurring units in the block copolymer are recurring units that comprise the first and second subunits. In other embodiments directed to a block copolymer that includes a first subunit and a second subunit, the block copolymer can include from about 25 mole % to about 75 mole % of recurring units that comprise the first subunit, based on the ratio of total moles of recurring units that comprise the first subunit to the total moles of recurring units in the block copolymer; and from about 25 mole % to about 75 mole % of recurring units that comprise the second subunit, based on the ratio of total moles of recurring units that comprise the second subunit to the total moles of recurring units in the block copolymer; and wherein about 98 mole % to about 99 mole % of the total moles of recurring units in the block copolymer are recurring units that comprise the first and second subunits.

In some embodiments, the block copolymer can include at least about 50 total recurring units. In some embodiments, the block copolymer can include at least about 100 total recurring units. In some embodiments, the block copolymer can include at least about 200 total recurring units. In some embodiments, the block copolymer can include at least about 500 total recurring units. In some embodiments, the block copolymer can include at least about 1000 total recurring units. In some embodiments, the block copolymer can include at least about 2000 total recurring units. In some embodiments, the block copolymer can include at least about 3000 total recurring units. In some embodiments, the block copolymer can include at least about 4000 total recurring units. In some embodiments, the block copolymer can include at least about 5000 total recurring units.

In some embodiments, the block copolymer can include from about 50 to about 5000 total recurring units. In other embodiments, the block copolymer can include from about 200 to about 1500 total recurring units. In other embodiments, the block copolymer can include from about 300 to about 700 total recurring units. In other embodiments, the block copolymer can include from about 2000 to about 4000 total recurring units. In yet other embodiments, the block copolymer can include from about 1000 to about 3000 total recurring units. In other embodiments, the block copolymer can include from about 50 to about 100 total recurring units, from about 1500 to about 2000 total recurring units, from about 1000 to about 1500 total recurring units, or from about 700 to about 1000 total recurring units. In yet other embodiments, the block copolymer can include from about 100 to about 200, from about 100 to about 500, or from about 300 to about 600 total recurring units.

The weight average molecular weight of the block copolymer can vary widely. In some embodiments, the weight average molecular weight of the block copolymer can be in the range of from about 8 kDa to about 200 kDa. In other embodiments, the weight average molecular weight of the block copolymer can be in the range of from about 50 kDa to about 150 kDa. In other embodiments, the weight average molecular weight of the block copolymer can be in the range of from about 75 kDa to about 125 kDa.

Those skilled in the art appreciate that varying the mole percentages of the first and second subunits relative to the total number of moles of subunits in the block copolymer can affect one or more properties of the block polymer as a whole. Examples of these properties include, but are not limited to, solubility, degradability, transfection efficiency, and toxicity. Those skilled in the art also appreciate that the relative mole percentages of the first and second subunits can be varied depending on the particular recurring units being incorporated into the first and second subunits. In addition, a combination of different recurring units as disclosed herein and/or additional recurring units having other structures and/or properties may be included in additional subunits of the block copolymers described herein depending on the desired characteristics of the block copolymer. The additional subunits and/or recurring units may be selected based on information available to those skilled in the art.

The block copolymers described herein can be made in a variety of ways. In some embodiments, a first reactant that includes a first polymer that forms the first subunit can be intermixed with a second reactant that includes a second polymer that forms the second subunit. In other embodiments, a first reactant that includes a first polymer that forms the first subunit can be intermixed with a second reactant that includes a monomer. In some embodiments, a monomeric reactant that forms a recurring unit of Formula (E) or a recurring unit of Formula (F) can be polymerized via RAFT polymerization to create a first polymer that can act as a macro chain transfer agent (CTA). The first polymer can then be intermixed with one or more monomeric reactants that form one or more recurring units of the second subunit. The monomeric reactants can be polymerized by RAFT polymerization, wherein the first polymer functions as the CTA, thereby yielding a block copolymer.

In other embodiments, a monomeric reactant that forms a recurring unit of the second subunit can be polymerized via RAFT polymerization to create a first polymer that can act as a macro chain transfer agent (CTA). The first polymer can then be intermixed with one or more additional monomeric reactants to form the copolymer of the second subunit. The copolymer can then be intermixed with a first polymer that includes a recurring unit of Formula (E) or a recurring unit of Formula (F) to yield a block copolymer.

In other embodiments, a monomeric reactant that forms the recurring unit of Formula (E) or the recurring unit of Formula (F) can be polymerized to form the first polymer. For example, the monomeric reactant can be HPMA or polyethylene glycol methacrylate (PEGMA). The monomeric reactant can be polymerized by RAFT polymerization to form the first polymer. The first polymer can then be coupled with a second polymer that can include one or more recurring units described herein (for example, a polymer that can include a recurring unit of Formula (I) and a recurring unit of Formula (II) as described herein; a polymer that can include a recurring unit of Formula (I), a recurring unit of Formula (II), and a recurring unit of Formula (III) as described herein; a polymer that can include a recurring unit of Formula (I), a recurring unit of Formula (II), and a recurring unit of Formula (A) as described herein; a polymer that can include a recurring unit of Formula (I), a recurring unit of Formula (II), and a recurring unit of Formula (XIII) as described herein; a polymer that can include a recurring unit of Formula (I), a recurring unit of Formula (II), a recurring unit of Formula (III), and a recurring unit of Formula (A) as described herein; a polymer that can include a recurring unit of Formula (I), a recurring unit of Formula (II), a recurring unit of Formula (III), and a recurring unit of Formula (XIII) as described herein; a polymer that can include a recurring unit of Formula (I), a recurring unit of Formula (II), a recurring unit of Formula (A), and a recurring unit of Formula (XIII) as described herein; and/or a polymer that can include a recurring unit of Formula (I), a recurring unit of Formula (II), a recurring unit of Formula (III), a recurring unit of Formula (A), and a recurring unit of Formula (XIII))). The first polymer and the second polymer can be coupled together using methods known to those skilled in the art, such as via carbodiimide chemistry.

In some embodiments, a nucleic acid can be incorporated into the block copolymer. The nucleic acid can be associated with the block copolymer in a variety of ways. In some embodiments, the nucleic acid can be selected from DNA, RNA, siRNA, and antisense. In some embodiments, the nucleic acid can be siRNA. In some embodiments, the nucleic acid can be associated with at least one recurring unit of Formula (III). In other embodiments, the nucleic acid can be associated with at least one recurring unit of Formula (E). For example, the nucleic acid can be associated with the terminal hydroxyl group of a recurring unit of Formula (III) or a recurring unit of Formula (E). In yet other embodiments, the nucleic acid can be associated with at least one recurring unit of Formula (I). For example, the nucleic acid can be associated with the terminal NR¹R²R^{5a} group of a recurring unit of Formula (I). In some embodiments, the nucleic acid can be associated with the first subunit and/or the second subunit via an electrostatic bond. In other embodiments, the nucleic acid can be associated with the first subunit and/or the second subunit via a covalent bond. In some embodiments where the nucleic acid is associated via a covalent bond, the nucleic acid can be directly covalently bonded to the first subunit and/or the second subunit of the block copolymer. In other embodiments, the nucleic acid can be indirectly bonded to the first subunit and/or the second subunit through a linking group. Examples of linking groups are described herein.

The polymers described herein that are associated with a nucleic acid (e.g., siRNA) can be administered to a human patient per se, or in pharmaceutical compositions where they are mixed with one or more suitable pharmaceutical carriers and/or excipient(s). Some embodiments herein provide a pharmaceutical composition that can include one or more polymers described herein associated with a nucleic acid, and further include at least one selected from a pharmaceutically acceptable excipient, a pharmaceutical carrier, and a diluent.

The term “pharmaceutical composition” refers to a mixture of a polymer associated with a nucleic acid disclosed herein with one or more other chemical components, such as diluents or additional pharmaceutical carriers. The pharmaceutical composition facilitates administration of the polymer and/or the nucleic acid to an organism.

Multiple techniques of administering a pharmaceutical composition exist in the art. Suitable routes of administration may include, for example, parenteral delivery, including intramuscular, subcutaneous, intravenous, intramedullary injections, as well as intrathecal, direct intraventricular, intraperitoneal, intranasal, or intraocular injections. The polymer associated with a nucleic acid can also be administered in sustained or controlled release dosage forms, including depot injections, osmotic pumps, and the like, for prolonged and/or timed, pulsed administration at a predetermined rate. Additionally, the route of administration may be local or systemic.

The term “pharmaceutical carrier” refers to a chemical compound that facilitates the incorporation of a polymer associated with a nucleic acid into cells or tissues.

The term “diluent” refers to chemical compounds diluted in water that will dissolve the polymer with an associated nucleic acid as well as stabilize the biologically active form of the polymer with the associated nucleic acid. Salts dissolved in buffered solutions are utilized as diluents in the art. As used herein, an “excipient” refers to an inert substance that is added to a polymer with an associated nucleic acid to provide, without limitation, bulk, consistency, stability, binding ability, lubrication, disintegrating ability, etc., to the composition. A “diluent” is a type of excipient.

The term “physiologically acceptable” refers to a pharmaceutical carrier or diluent that does not abrogate the biological activity and properties of the polymer or the nucleic acid.

Techniques for formulation and administration of the pharmaceutical compositions of the instant application may be found in “Remington's Pharmaceutical Sciences,” Mack Publishing Co., Easton, Pa., 18th edition, 1990. The pharmaceutical compositions may be manufactured in a manner that is itself known. Pharmaceutical compositions may be formulated in any conventional manner using one or more physiologically acceptable pharmaceutical carriers comprising excipients and auxiliaries which facilitate processing of the active compounds into preparations which can be used pharmaceutically. Proper formulation is dependent upon the route of administration chosen. Any of the well-known techniques, pharmaceutical carriers, and excipients may be used as suitable and as understood in the art; e.g., in Remington's Pharmaceutical Sciences, above.

Some embodiments herein are directed to a method of delivering a nucleic acid such as siRNA to a cell. In other embodiments, the polymers described herein can be used to transfect a cell. These embodiments can include delivering the nucleic acid via the polymer to the cell, for example by contacting the cell with the polymer. Suitable cells for use according to the methods described herein include prokaryotes, yeast, or higher eukaryotic cells, including plant and animal cells (e.g., mammalian cells). In some embodiments, the cells can be tumor cells, such as animal (e.g., mammalian and/or human) tumor cells. Cells lines which are model systems for tumors may be used. In some embodiments these methods can be performed in vitro, while in other embodiments they can be performed in vivo.

Other embodiments are directed to a method of treating a mammal. These embodiments may include identifying a mammal in need of gene therapy and administering to the mammal a polymer associated with a nucleic acid as described herein.

Some embodiments disclosed herein are directed to a method for treating a tumor that can include administering an effective amount of a polymer associated with a nucleic acid as described herein. Other embodiments disclosed herein are directed to a method for treating a tumor that can include contacting a tumor cell with an effective amount of a polymer associated with a nucleic acid as described herein. Treatment of a tumor can include shrinking the tumor and/or killing or damaging some or all of the tumor cells. The tumor can be benign, pre-malignant, or malignant (e.g., cancerous). The tumor can be solid or non-solid (e.g., dispersed). Examples of solid tumors include, but are not limited to, those associated with lung cancer, breast cancer, ovarian cancer, prostate cancer, colorectal cancer, brain cancer, testicular cancer, pancreatic cancer, liver cancer, and stomach cancer. Other examples of solid tumors include, but are not limited to, sarcomas, carcinomas, melanomas, and lymphomas. In some embodiments, the nucleic acid can treat the tumor.

The pharmaceutical compositions described herein may be administered to the subject by any suitable means. Non-limiting examples of methods of administration include, among others, (a) administration via injection, subcutaneously, intraperitoneally, intravenously, intramuscularly, intradermally, intraorbitally, intracapsularly, intraspinally, intrasternally, or the like, including infusion pump delivery; (b) administration locally such as by injection directly in the renal or cardiac area, e.g., by depot implantation; as well as deemed appropriate by those of skill in the art for bringing the polymer associated with a nucleic acid into contact with living tissue.

Pharmaceutical compositions suitable for administration include polymers where the active ingredients (e.g., associated nucleic acid) are contained in an amount effective to achieve its intended purpose. The effective amount of the nucleic acids and polymers disclosed herein required as a dose will depend on the route of administration, the type of animal, including human, being treated, and the physical characteristics of the specific animal under consideration. The dose can be tailored to achieve a desired effect, but will depend on such factors as weight, diet, concurrent medication and other factors which those skilled in the medical arts will recognize. More specifically, a therapeutically effective amount means an amount of nucleic acid effective to prevent, alleviate or ameliorate symptoms of disease or prolong the survival of the subject being treated. Determination of a therapeutically effective amount is well within the capability of those skilled in the art, especially in light of the detailed disclosure provided herein.

It should be noted that the attending physician would know how to and when to terminate, interrupt, or adjust administration due to toxicity or organ dysfunctions. Conversely, the attending physician would also know to adjust treatment to higher levels if the clinical response were not adequate (precluding toxicity). The magnitude of an administrated dose in the management of the disorder of interest will vary with the severity of the condition to be treated and to the route of administration.

Polymers disclosed herein can be evaluated for efficacy and toxicity using known methods. For example, the toxicology of a particular compound, or of a subset of the compounds, sharing certain chemical moieties, may be established by determining in vitro toxicity towards a cell line, such as a mammalian, and preferably human, cell line. The efficacy of a particular compound may be established using several recognized methods, such as in vitro methods, animal models, or human clinical trials. Recognized in vitro models exist for nearly every class of condition, including but not limited to cancer, cardiovascular disease, and various immune dysfunction. When selecting a model to determine efficacy, the skilled artisan can be guided by the state of the art to choose an appropriate model, dose, and route of administration, and regime.

### EXAMPLES

Additional embodiments are disclosed in further detail in the following examples, which are not in any way intended to limit the scope of the claims. All chemicals and other reagents were purchased from Sigma-Aldrich.

### Example 1

### Polymer Synthesis

### Synthesis of N-(3-Aminopropyl)methacrylamide homopolymer

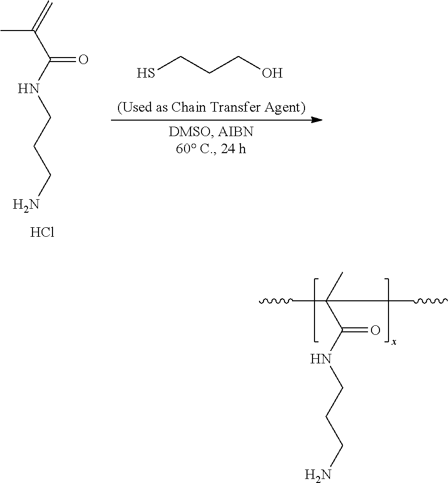

N-(3-Aminopropyl)methacrylamide hydrochloride (6.7 g, 37.5 mmol), an initiator, 2,2′-azobisisobutyronitrile (AIBN, 0.244 g, 3 mol %), and a chain transfer agent, mercaptopropanol (194 μL, 6 mol %), were dissolved in DMSO (40 mL, 15 wt %), placed in an ampoule, and purged with N₂ for 5 min. The free radical polymerization reaction was allowed to proceed at 60° C. for 24 hours. The resulting polymer was dissolved in water and dialyzed for 48 h using a 1000 molecular weight cut off MWCO) membrane to remove any unreacted monomers. The polymer was isolated by freeze drying. The yield of polymer was 70%.

### Synthesis of N-(2-Hydroxypropyl)methacrylamide-co-N-(3-Aminopropyl)methacrylamide Copolymer

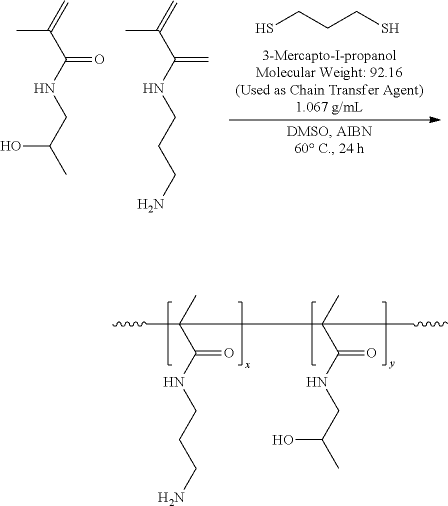

N-(3-Aminopropyl)methacrylamide hydrochloride (5 g, 28.0 mmol), N-(2-Hydroxypropyl)methacrylamide (1 g, 6.98 mmol), AIBN (0.227 g, 3 mol %) and mercaptopropanol (180 μL, 6 mol %) were dissolved in DMSO (35 mL, 15 wt %), placed in an ampoule, purged with N₂ for 5 min, and sealed. The free radical copolymerization reaction was allowed to proceed at 60° C. for 24 hours. The copolymer was dissolved in water and dialyzed for 48 h using a 1000 MWCO membrane to remove any unreacted monomers. The copolymer was isolated by freeze drying. The yield of the copolymer was 4.1 g (68.5%).

### Attachment of Lipid to N-(3-Aminopropyl)methacrylamide homopolymer

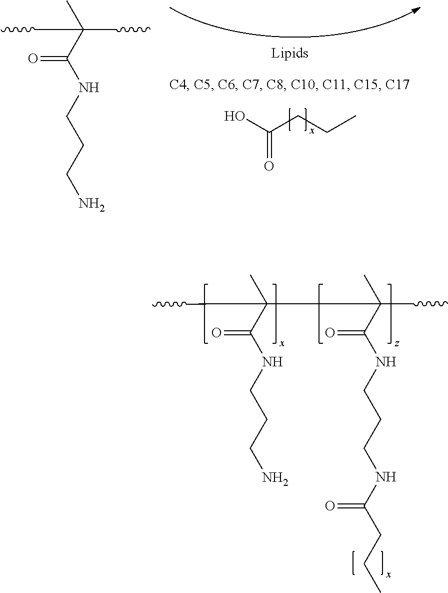

Lipids of different lengths (e.g., optionally substituted C₄-C₁₈ fatty acids) were attached to a poly(N-(3-aminopropyl)methacrylamide polymer (synthesized as described above) in DMSO at room temperature for 24 h with stirring. In an example, nananoic acid (27.6 mg, 0.17 mmol), N-(3-Aminopropyl)methacrylamide homopolymer (100 mg, 0.559 mmol [NH₂]), N,N-Diisopropylethylamine (60 μL, 0.34 mmol) and N,N′-diisopropylcarbodiimide (54.67 μL, 0.34 mmol) were intermixed at room temperature for 24 h with stirring. The polymer was dissolved in water and dialyzed for 48 h using a 1000 MWCO membrane (to remove unreacted monomer). The polymer was isolated by freeze drying.

### Attachment of Lipid to N-(2-Hydroxypropyl)methacrylamide-co-N-(3-Aminopropyl)methacrylamide Copolymer

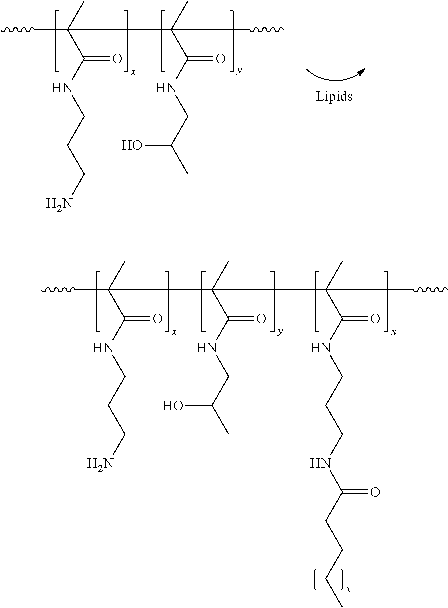

Lipids of different lengths (e.g., optionally substituted C₄-C₁₈ fatty acids) were attached to a copolymer in DMSO at room temperature for 24 h with stirring in a manner similar to the attachment of a lipid to a polymer as described above, with N-(2-Hydroxypropyl)methacrylamide-co-N-(3-Aminopropyl)methacrylamide copolymer instead of poly(N-(3-Aminopropyl)methacrylamide) homopolymer. In an example, 100 mg (0.45 mmol [NH₂]) of N-(2-Hydroxypropyl)methacrylamide-co-N-(3-Aminopropyl)methacrylamide copolymer was used.

### Attachment of Lipid & pKa modulator to N-(3-Aminopropyl)methacrylamide Homopolymer

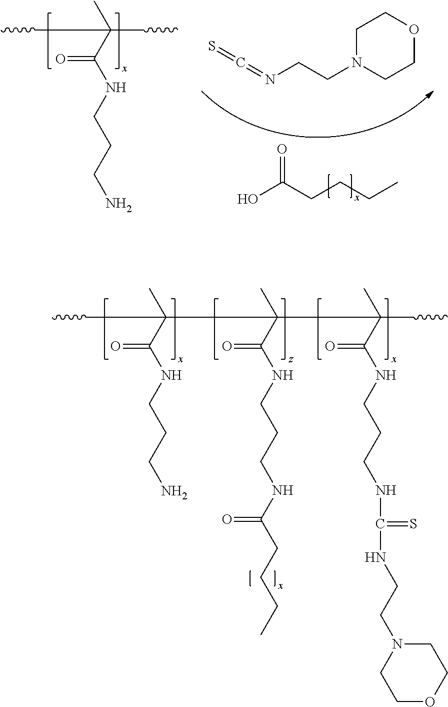

Lipids of different lengths (e.g., optionally substituted C₄-C₁₈ fatty acids) and moieties having a pH transition point were attached to a polymer in DMSO at room temperature for 24 h with stirring. In an example, nananoic acid (27.6 mg, 0.17 mmol), N-(3-Aminopropyl)methacrylamide homopolymer (100 mg, 0.559 mmol [NH₂]), 4-(2-isothiocyanatoethyl)morpholine (21 mg, 0.14 mmol), N,N-Diisopropylethylamine (60 μL, 0.34 mmol) and N,N′-diisopropylcarbodiimide (54.67 μL, 0.34 mmol) were intermixed at room temperature for 24 h with stirring. The polymer was dissolved in water and dialyzed for 48 h using a 1000 MWCO membrane to remove unreacted monomer. The polymer was isolated by freeze drying.

### Attachment of Lipid & pKa modulator to N-(2-Hydroxypropyl)methacrylamide-co-N-(3-Aminopropyl)methacrylamide copolymer

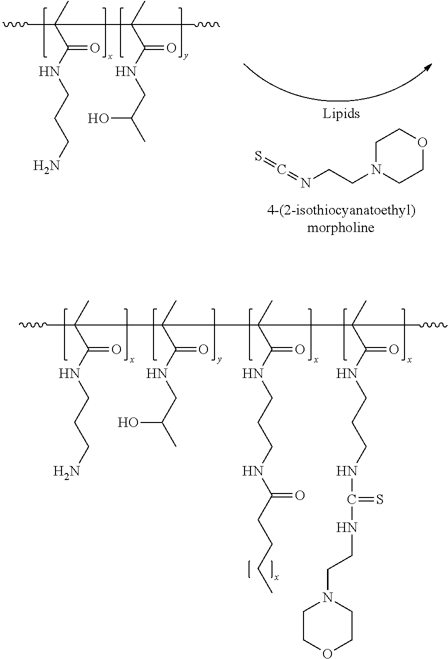

Lipids of different lengths (e.g., optionally substituted C₄-C₁₈ fatty acids) and moieties having a pH transition point were attached to a polymer in DMSO at room temperature for 24 h with stirring as described above, with N-(2-Hydroxypropyl)methacrylamide-co-N-(3-Aminopropyl)methacrylamide copolymer instead of N-(3-Aminopropyl)methacrylamide homopolymer. In an example, 100 mg (0.45 mmol [NH₂]) of N-(2-Hydroxypropyl)methacrylamide-co-N-(3-Aminopropyl)methacrylamide copolymer were used.

### Example 2

### Polymer Synthesis

### Polymer 1a

Monomers HPMA, morpholinoethyl methacrylate, n-octyl methacrylate, N-(3-aminopropyl)methacrylamide, and AIBN were weighed out in respective amounts of 85 mol %, 10 mol %, 1 mol %, and 4 mol %, based on the total moles of monomers, and transferred to a 20 mL vial. DMSO solvent was added to the vial in an amount of 85 wt % based on the total weight of the reaction mixture. A clear solution was formed after vortexing for 2 min. This solution was then transferred to an ampoule and purged with argon for 4 min. The ampoule was then sealed with a torch. The reaction mixture in the ampoule was heated at 60° C. for 15 hours until a thick viscous solution was formed. The ampoule was then broken. The thick viscous solution was then added to excess acetone to precipitate polymer 1a. Polymer 1a was filtered to obtain a dry powder. Polymer 1a was characterized by ¹H-NMR and gel permeation chromatography with a light scattering detector.

### Polymers 1b-28j

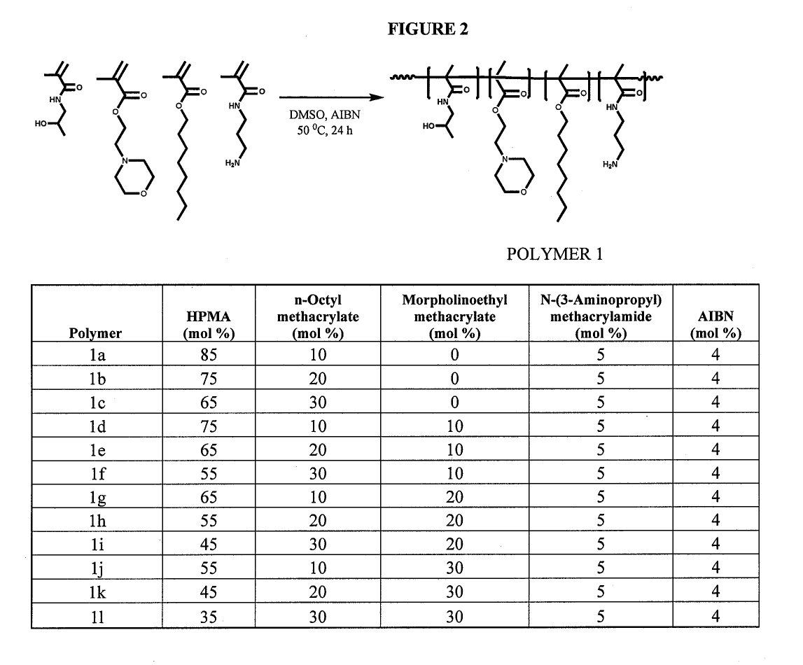

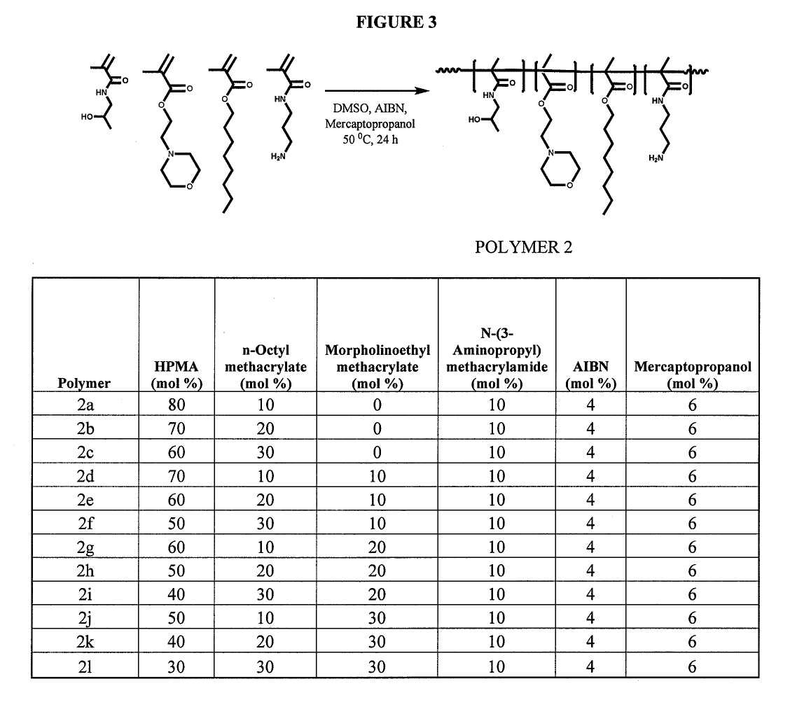

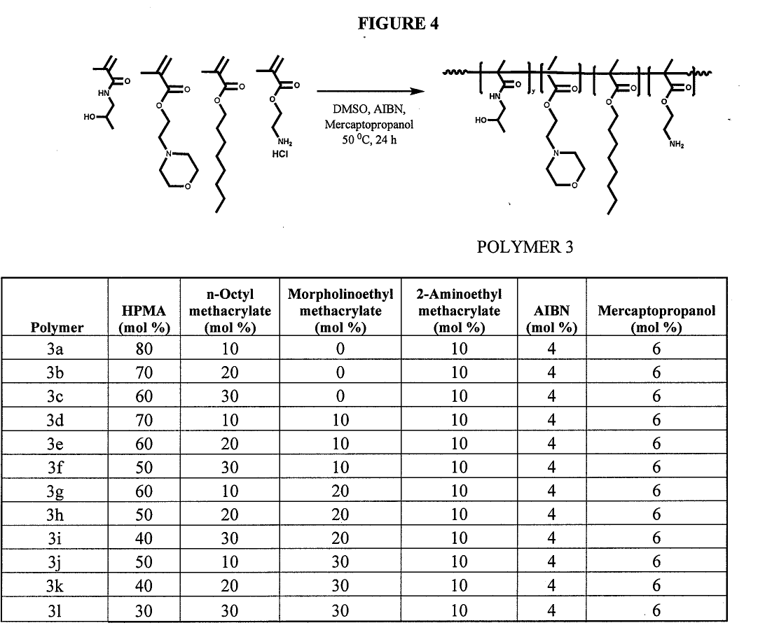

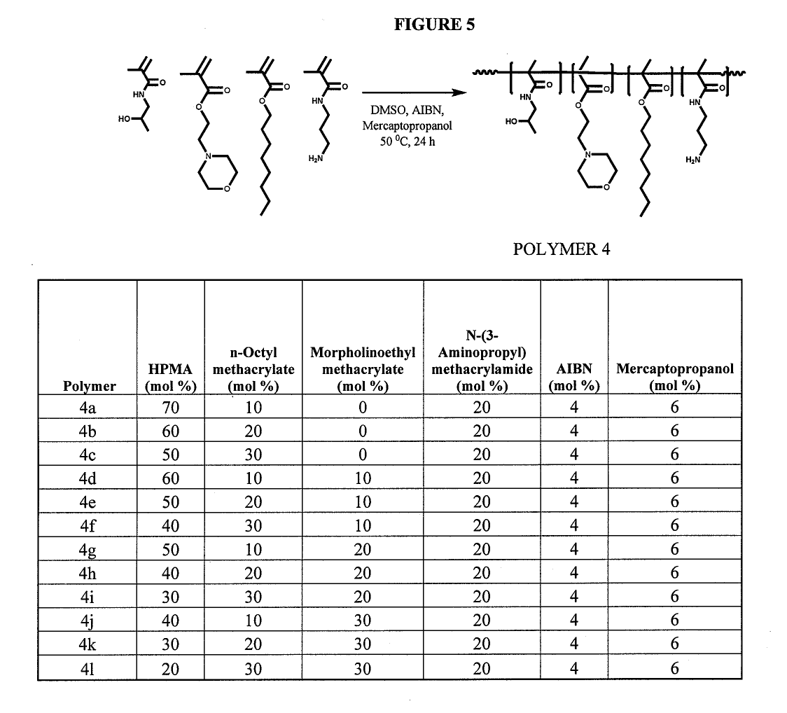

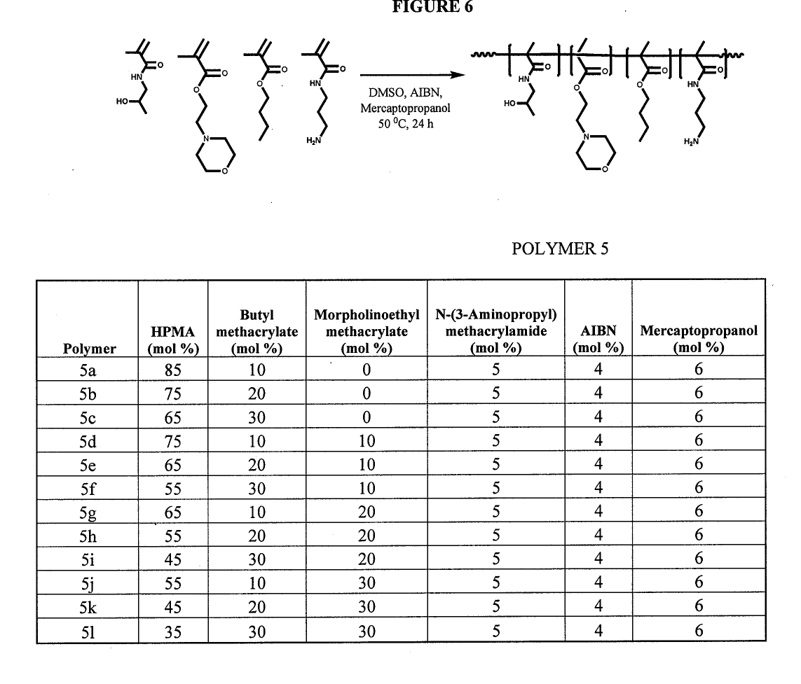

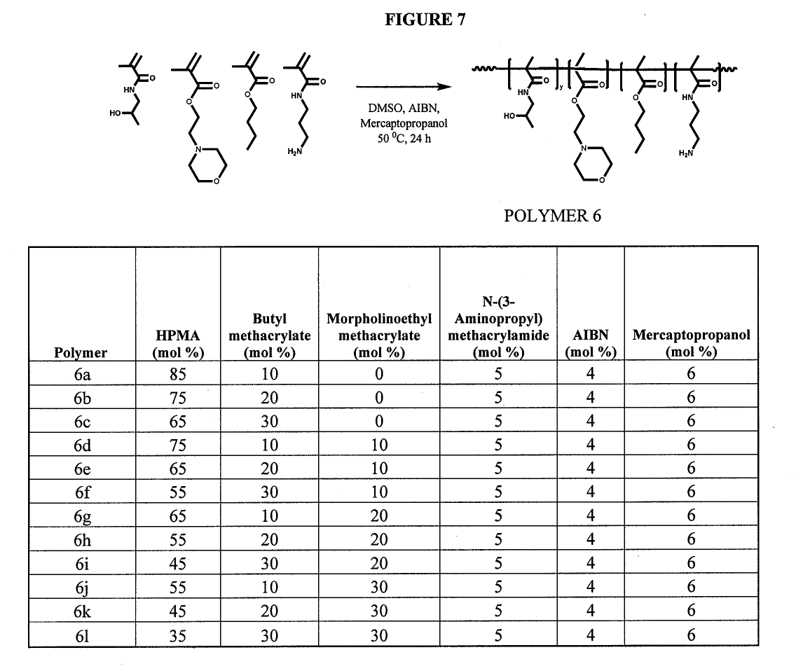

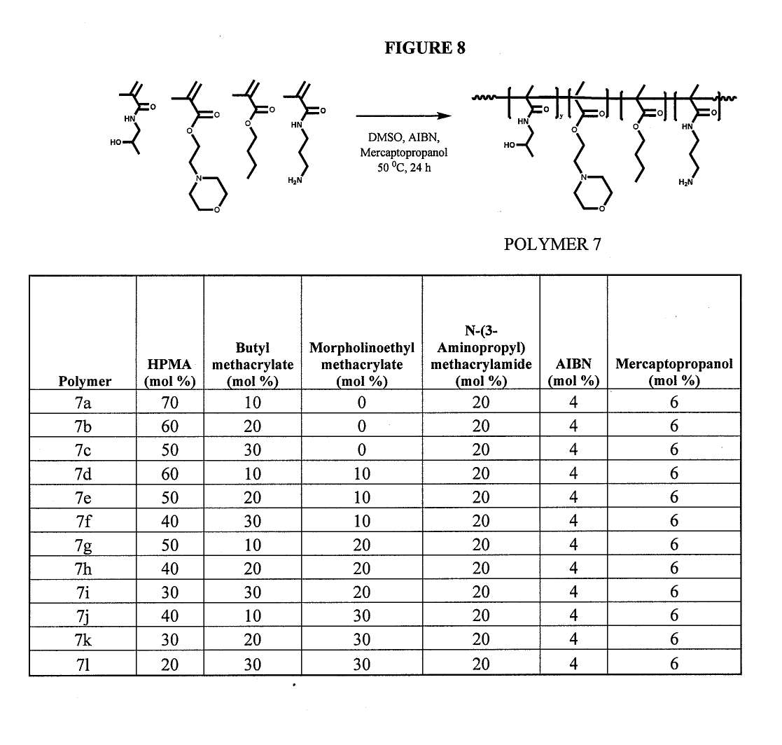

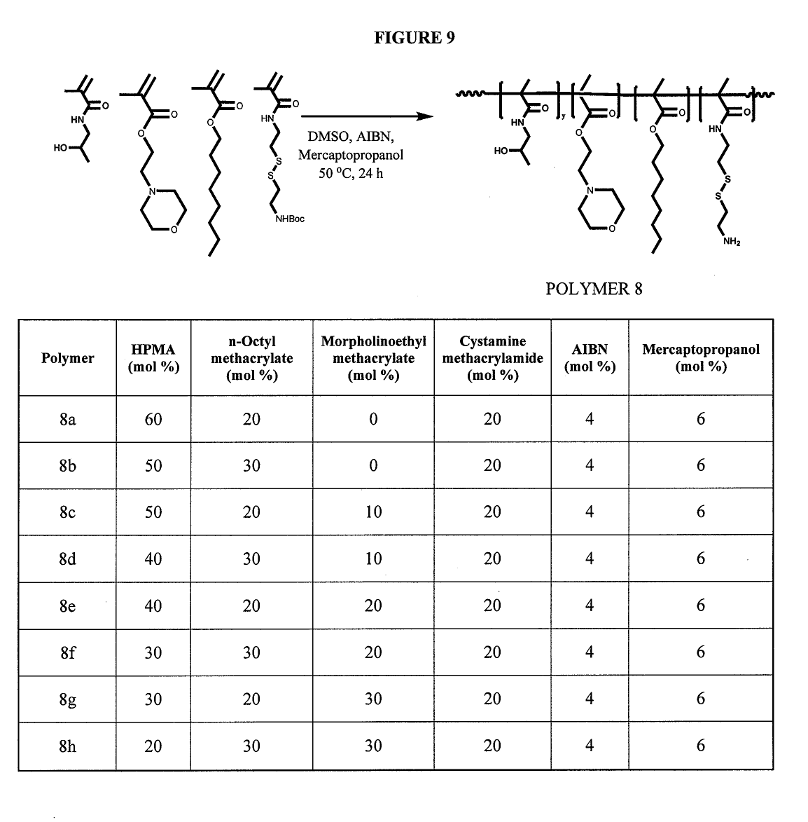

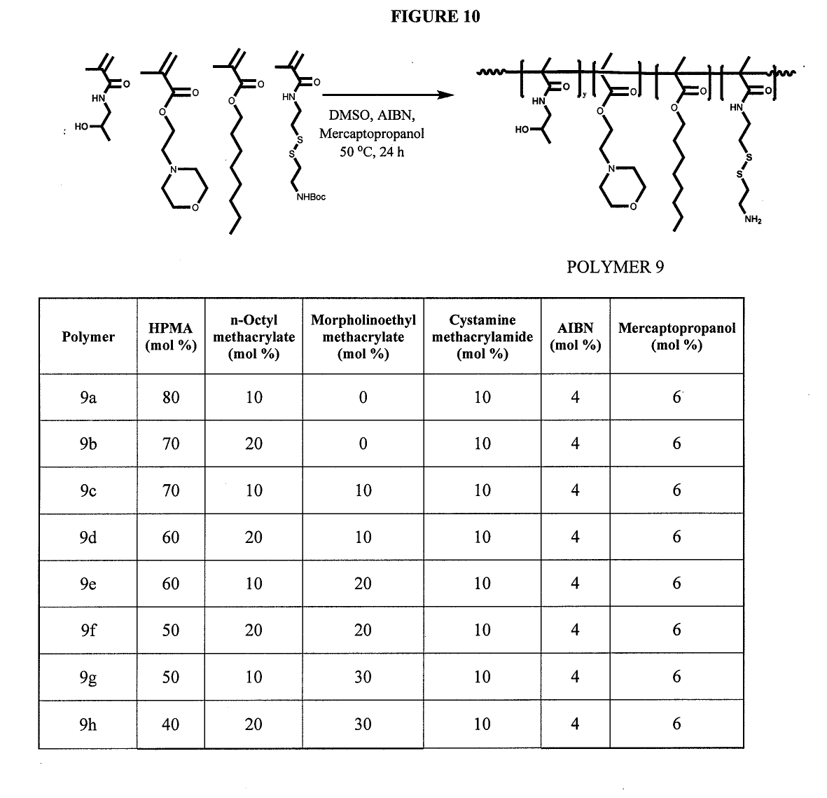

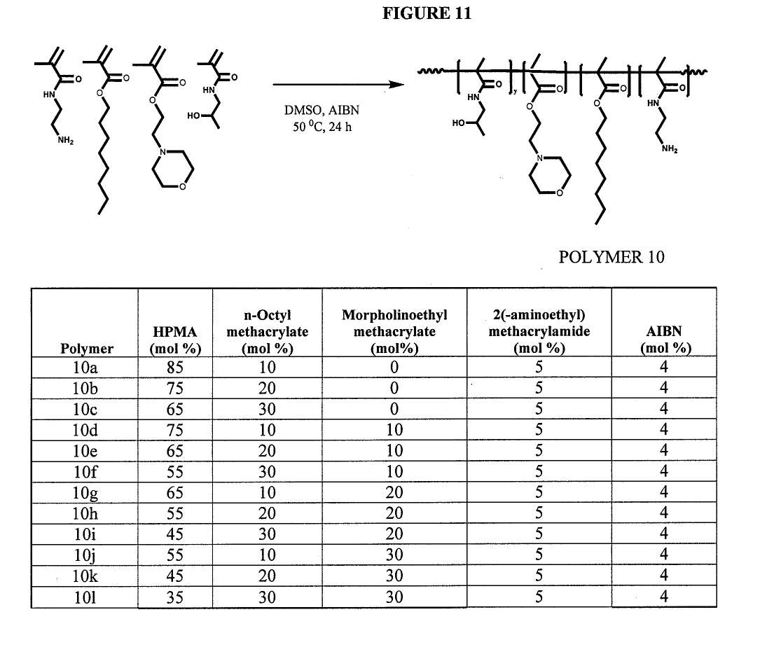

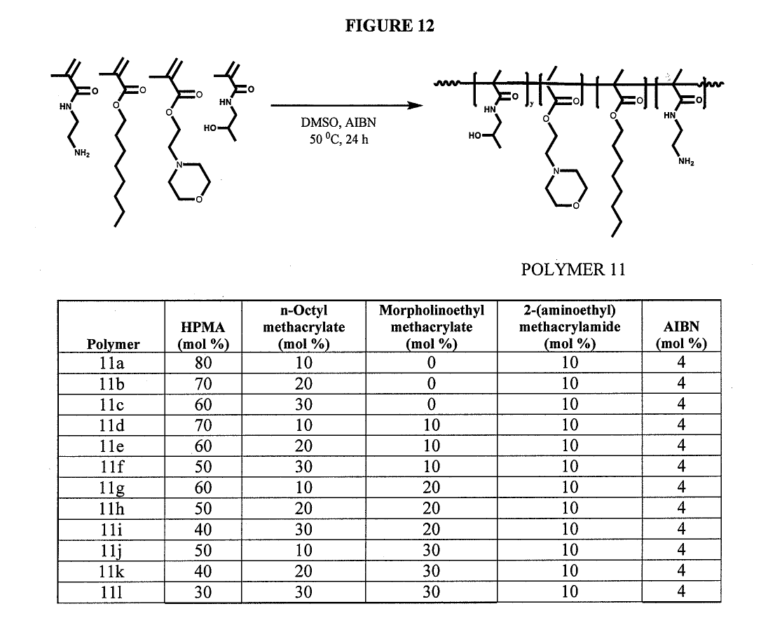

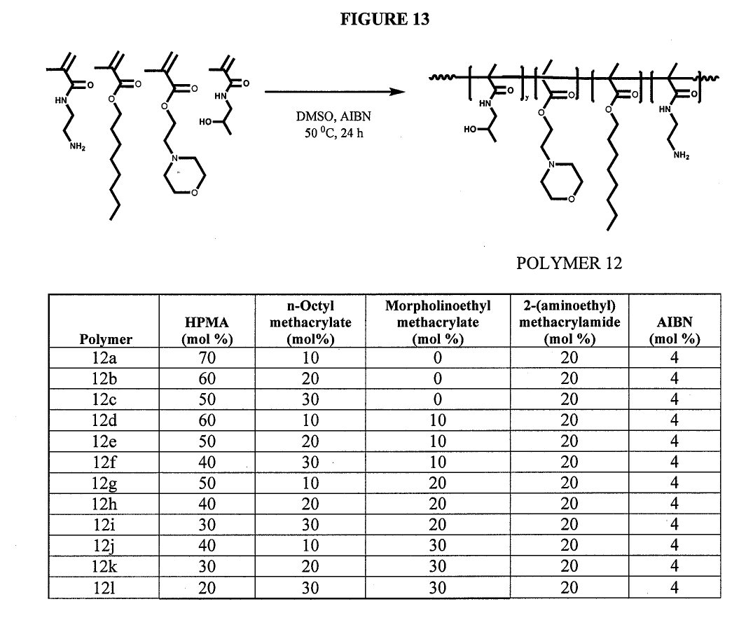

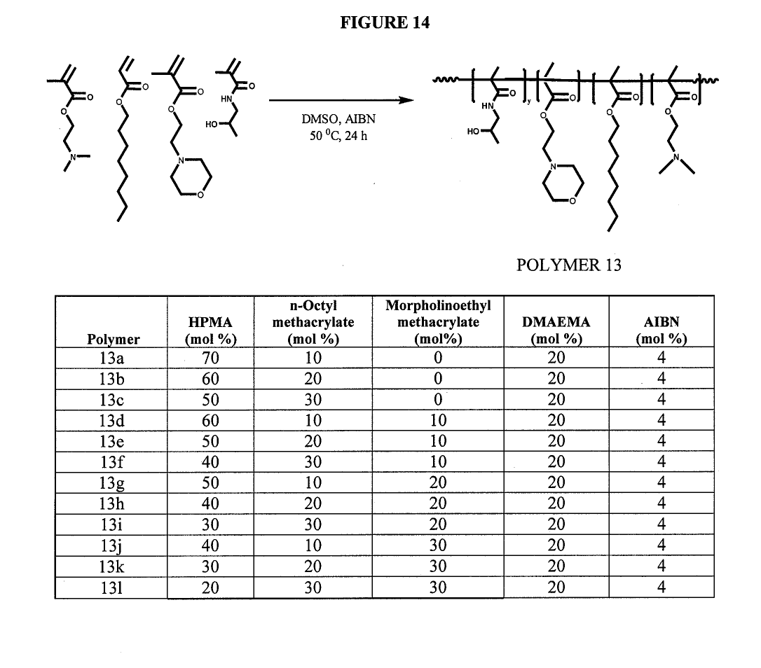

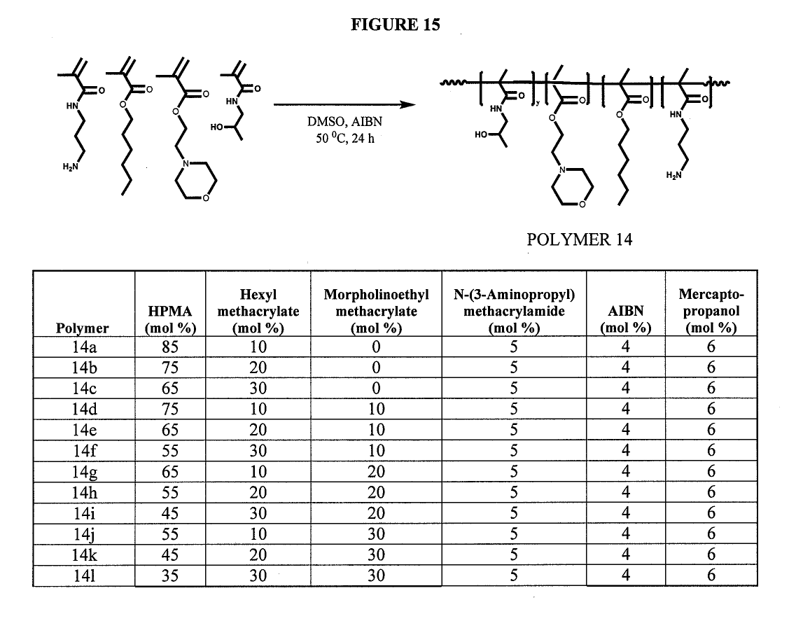

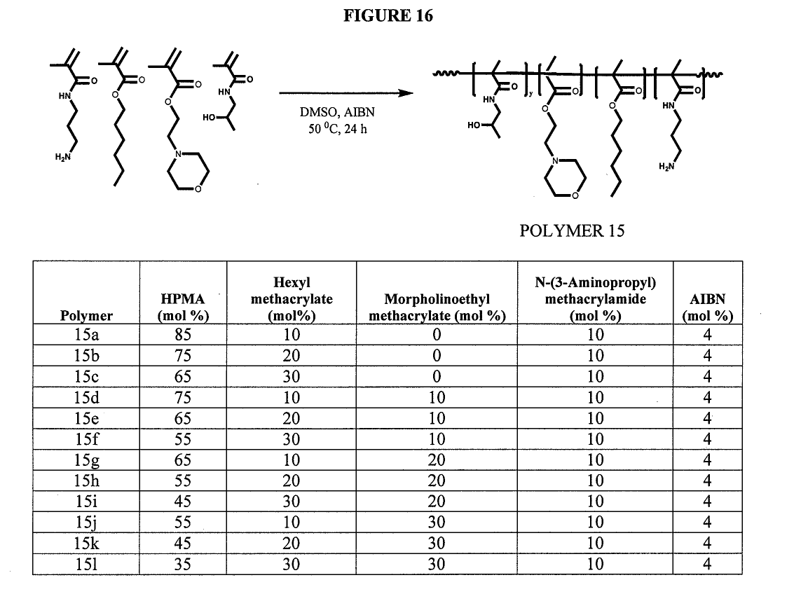

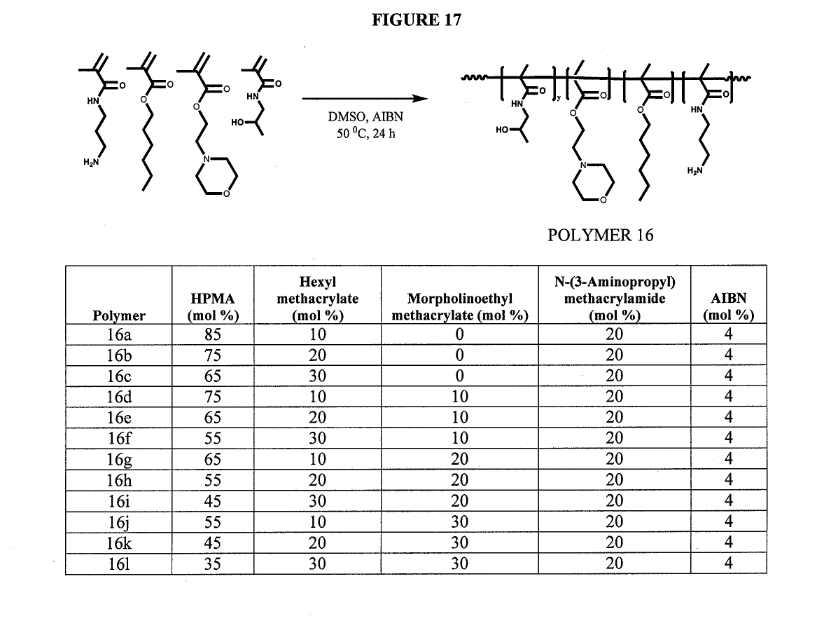

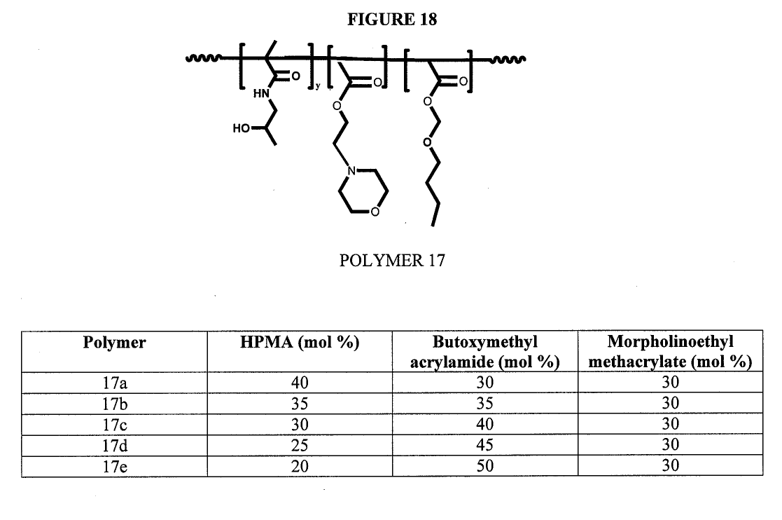

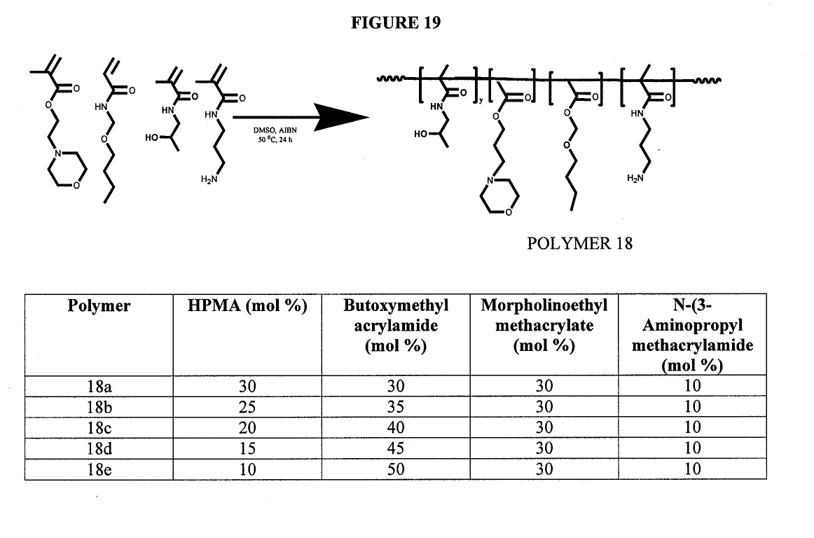

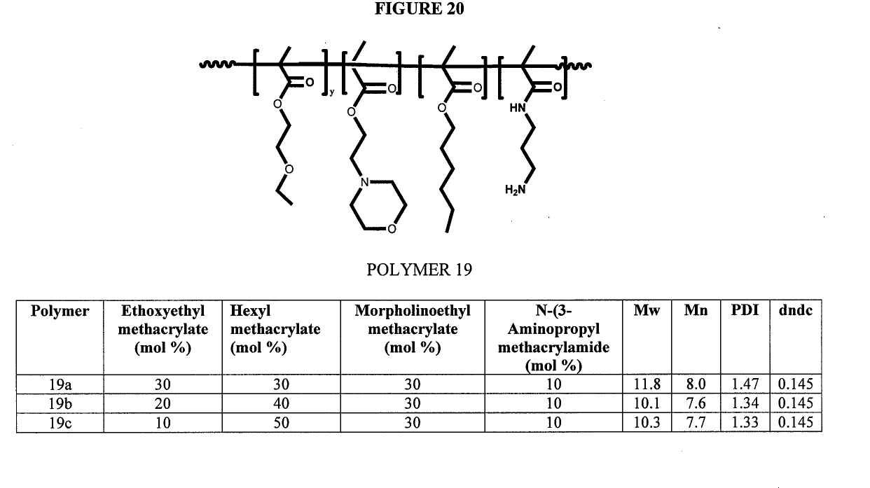

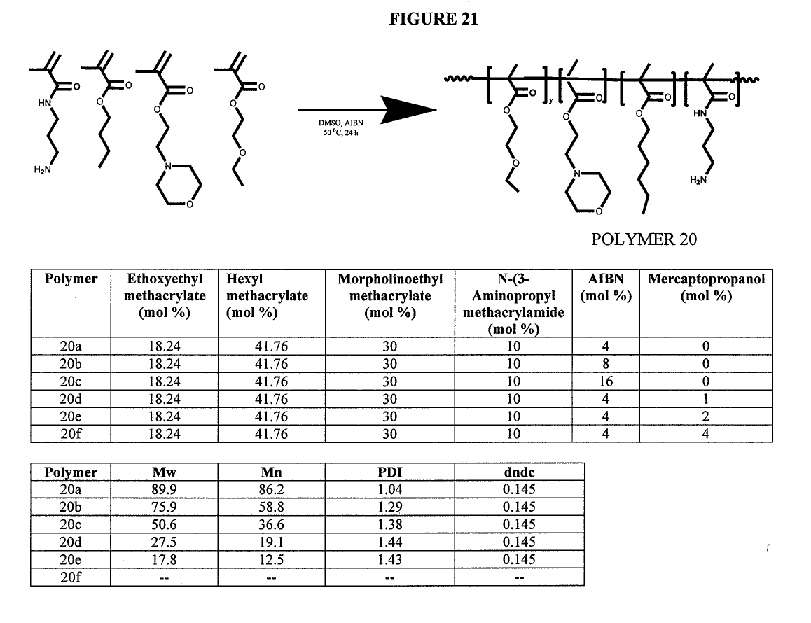

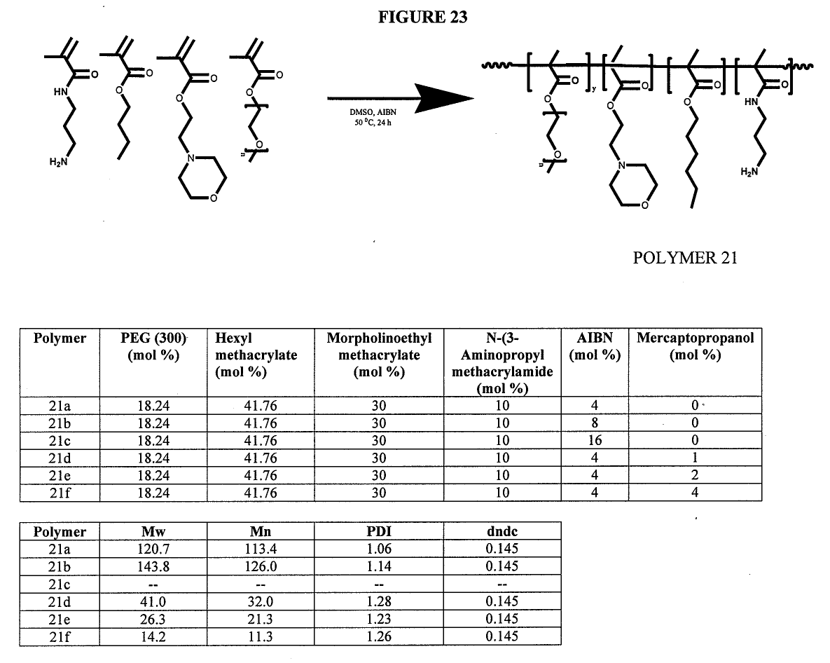

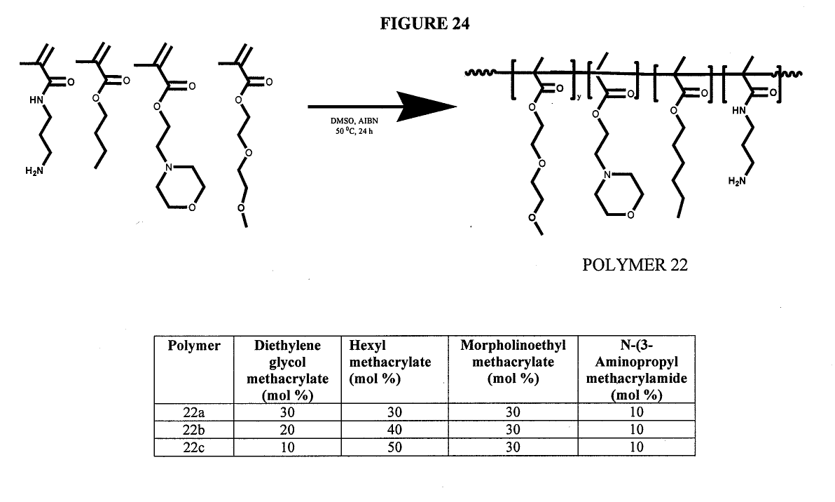

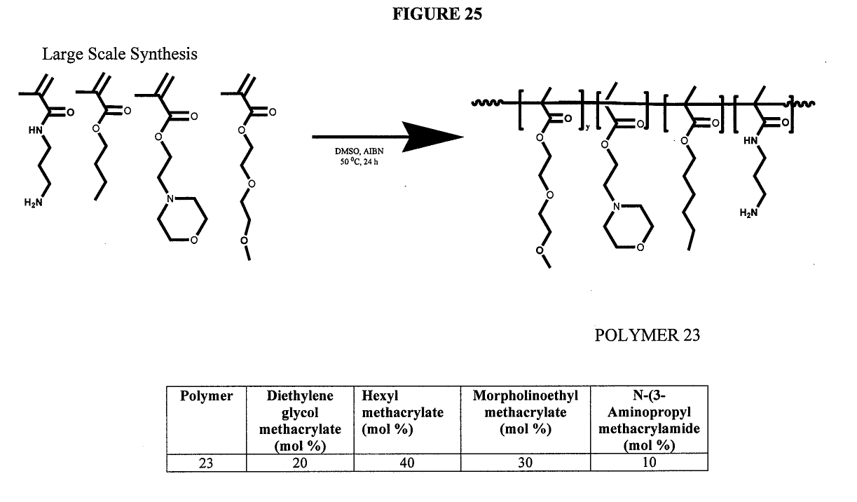

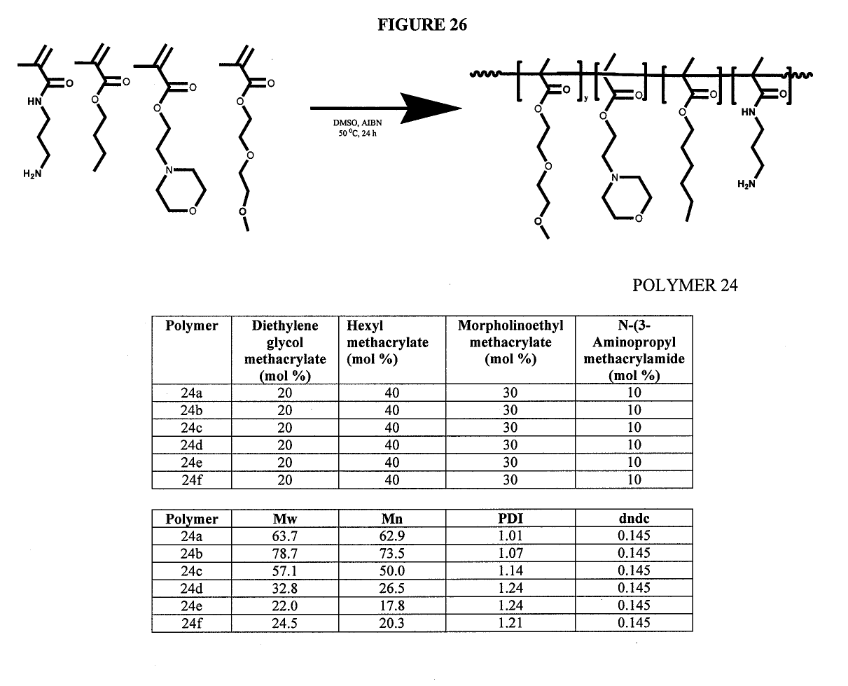

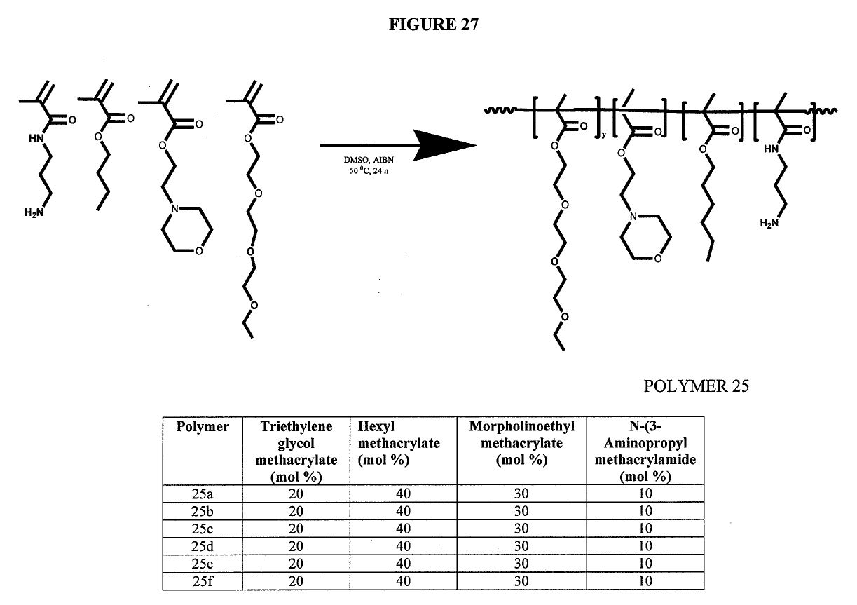

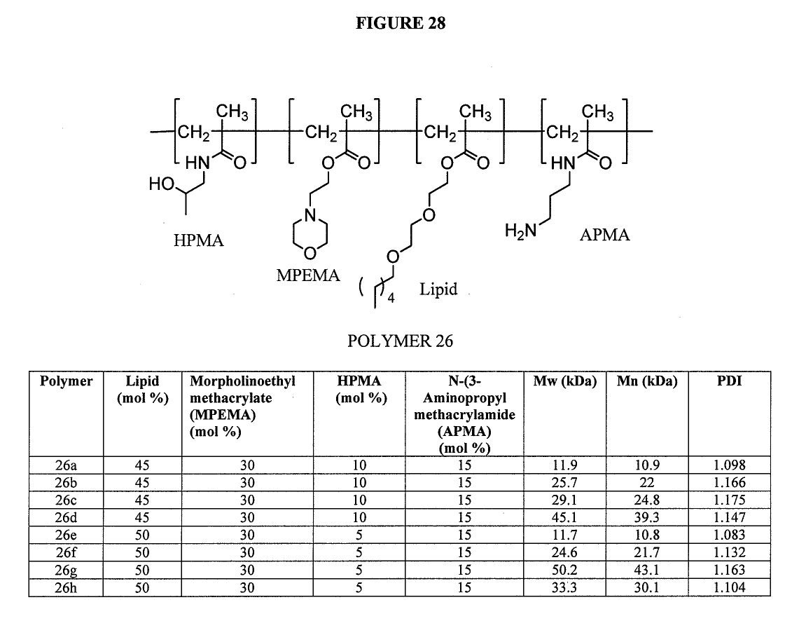

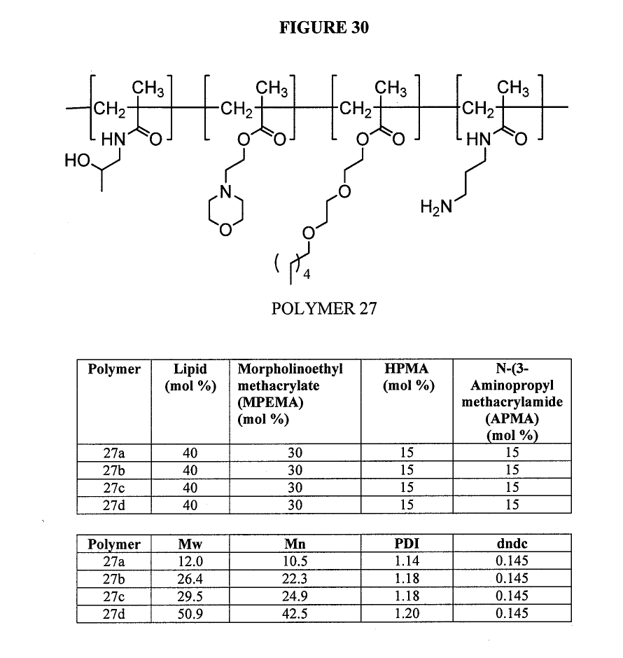

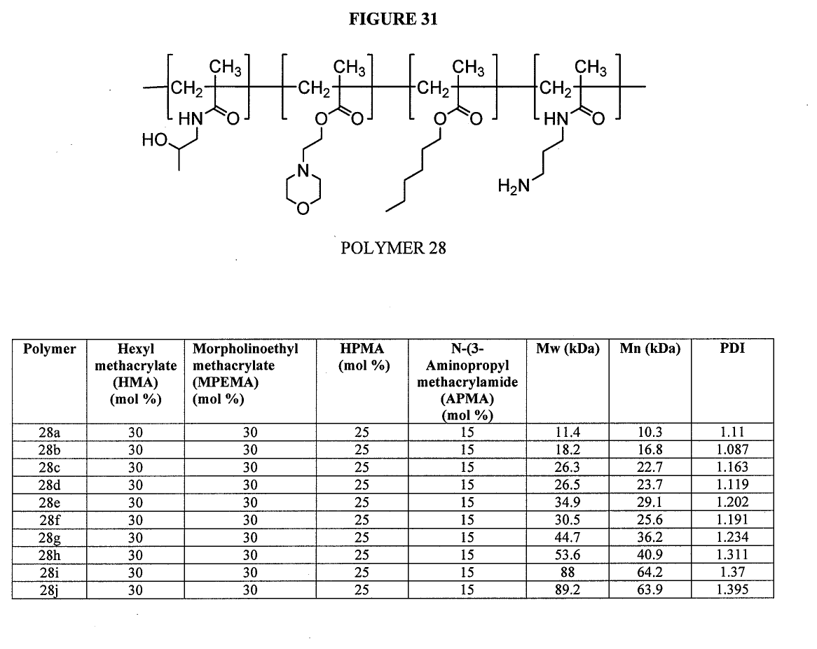

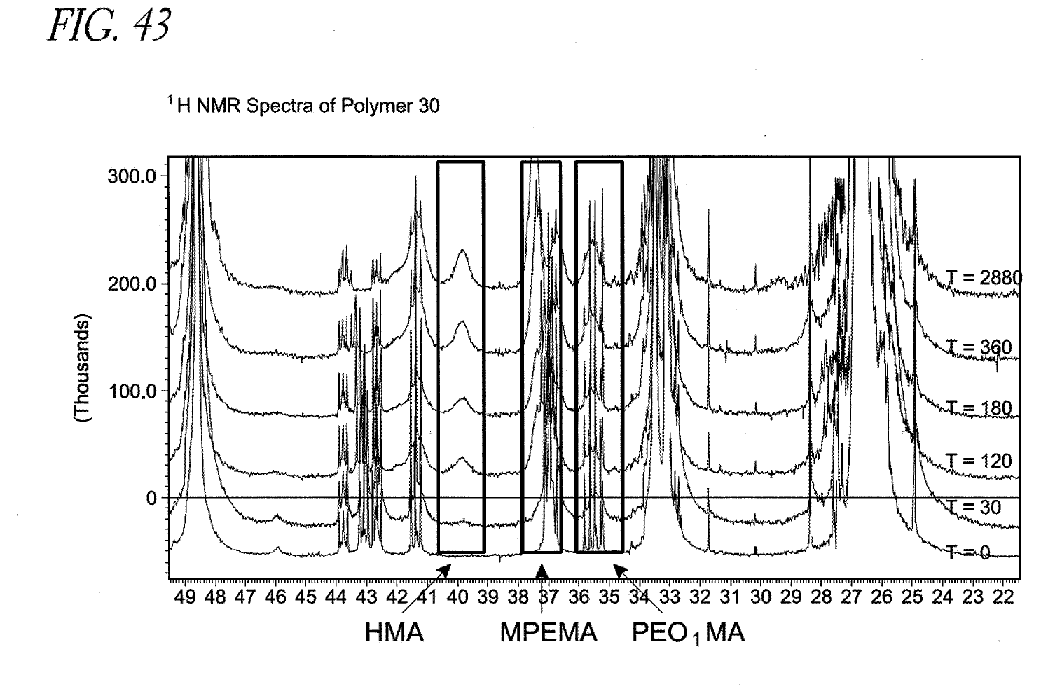

Polymers 1b-28j were synthesized in a similar manner as polymer 1a, but with the respective monomers and amounts specified in FIGS. 2-21, 23-28, and 30-31. Polymer 14 was characterized by ¹H-NMR during the polymerization process. Representative peaks indicating the formation of Polymer 14 include the following: δ 4.2-4.1 ppm (MPEMA); δ 4.1-3.9 ppm (HMA); δ 3.8-3.7 ppm (MPEMA); δ 3.3-3.1 (HPMA and APMA); and δ 3.0-2.9 (HPMA and APMA). See FIG. 42. As time increased (as measured along the y-axis), the peaks coalesced, indicating that the monomeric reactants had polymerized.

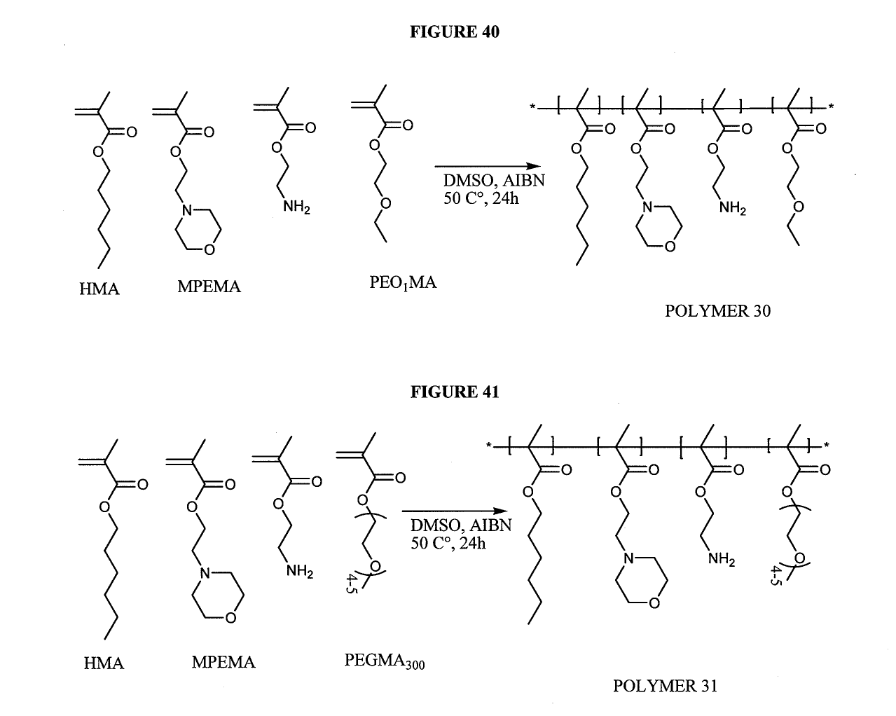

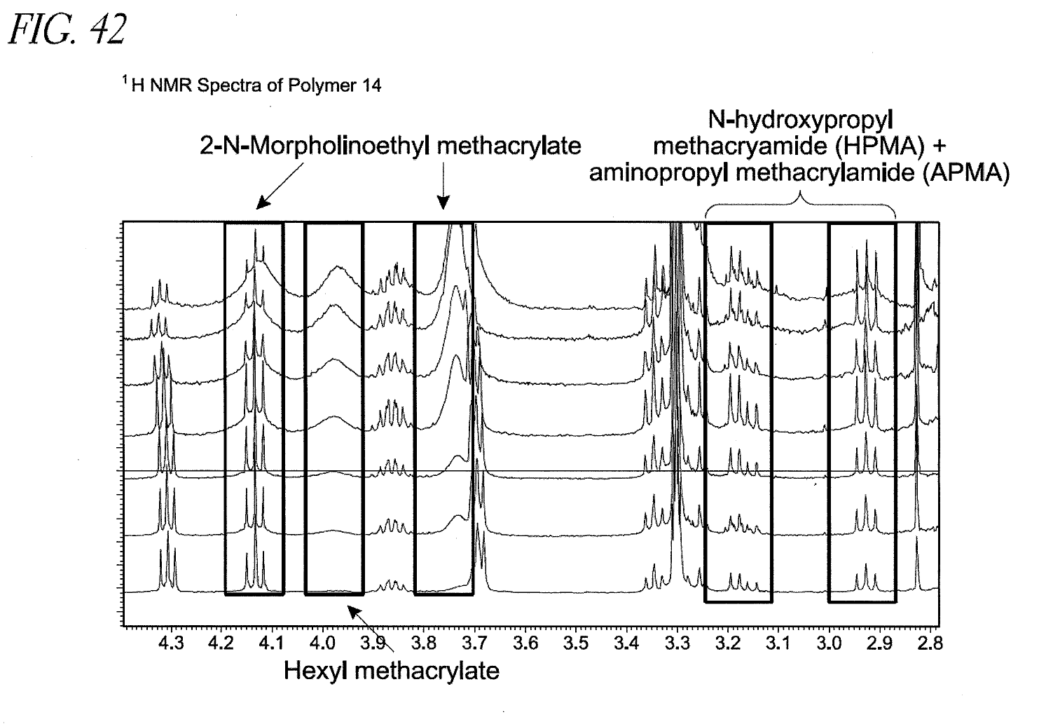

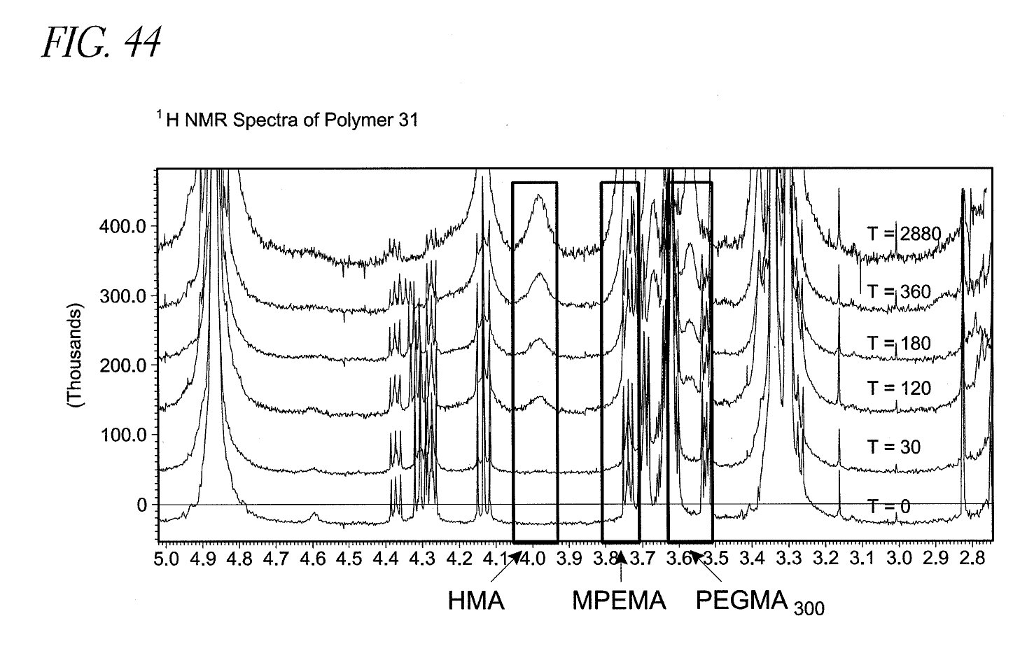

Polymers 30-31 were synthesized in a similar manner as polymer 1a, but with the respective monomers specified in FIGS. 40-41. Polymers 30-31 were characterized by ¹H-NMR during the polymerization process. Representative peaks indicating the formation of Polymer 30 include the following: δ 4.1-3.9 ppm (HMA); δ 3.8-3.7 ppm (MPEMA); and δ 3.6-3.4 ppm (PEO₁MA). See FIG. 43. Representative peaks indicating the formation of Polymer 31 include the following: δ 4.1-3.9 ppm (HMA); δ 3.8-3.7 ppm (MPEMA); and δ 3.6-3.5 ppm (PEGMA₃₀₀). See FIG. 44. As time (T) increased (as measured along the y-axis), the peaks coalesced, indicating that the monomeric reactants had polymerized.

### Example 3

### Block Copolymer Synthesis

### Synthesis of PEG-CTA

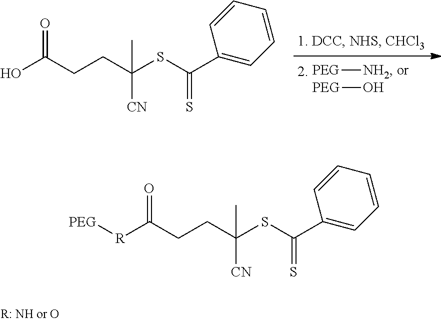

PEG-CTA (PEG-chain transfer agent) was synthesized using a carbodiimide coupling reaction. 4-cyanopentanoic acid dithiobenzoate (CTP) (1 eq, purchased from Aldrich) and NHS (2 eq) were dissolved in CHCl₃. DCC (6 eq) was added to the solution at room temperature. The reaction solution was continuously stirred overnight under an inert environment at room temperature. Dicyclohexylurea (DCU) was then filtered out, and the reaction mixture was washed with CHCl₃. The organic solvent was removed by evaporator to get the desired NHS-activated CTP, which can be used without further purification. The NHS-CTP (1.3 eq) was dissolved in MeOH. The NHS-CTP solution was added to a solution of PEG-NH₂ having a molecular weight (M_{w}) of 2 kDa, 5 kDa, or 10 kDa in MeOH (1 eq) over 5-10 minutes. The reaction mixture was stirred under an inert environment overnight at room temperature. The product was purified by dialysis against ultrapure water using a 1000 MWCO membrane over 48 h. The PEG-CTA was obtained by lyophilization.

### Synthesis of PEG-Block Copolymer

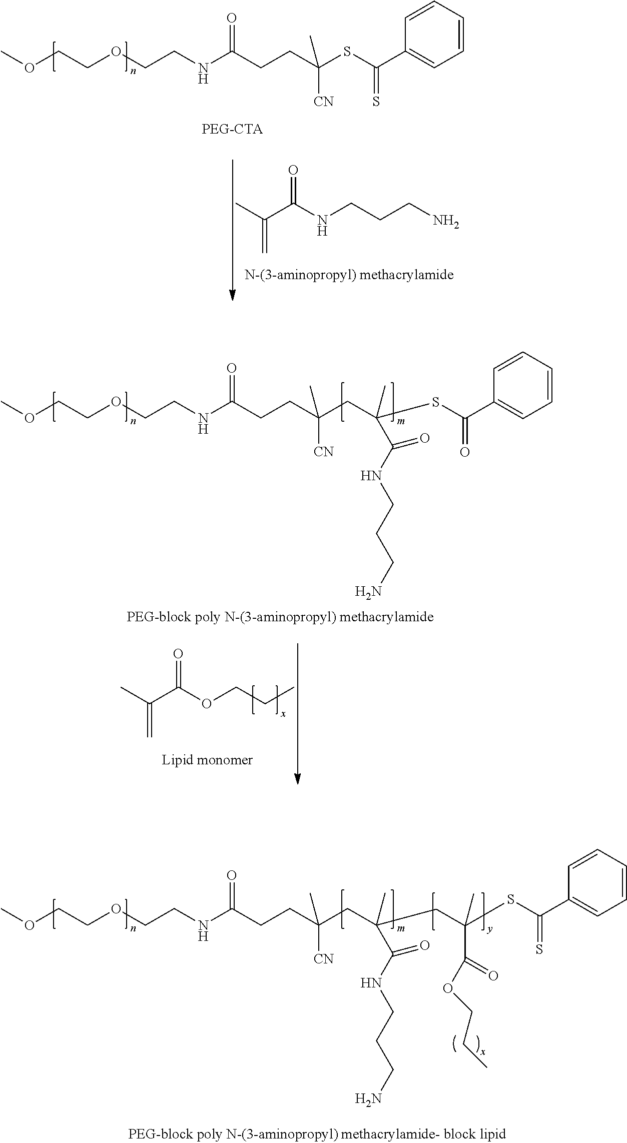

The PEG-CTA synthesized above was intermixed with a N-(3-aminopropyl)methacrylamide monomer to form a diblock copolymer that includes a PEG block (Block A) and a poly(N-(3-aminopropyl)methacrylamide) block (Block B). The diblock copolymer was intermixed with a lipid monomer to add a lipid unit to Block B.

### Example 4

### Block Copolymer Synthesis

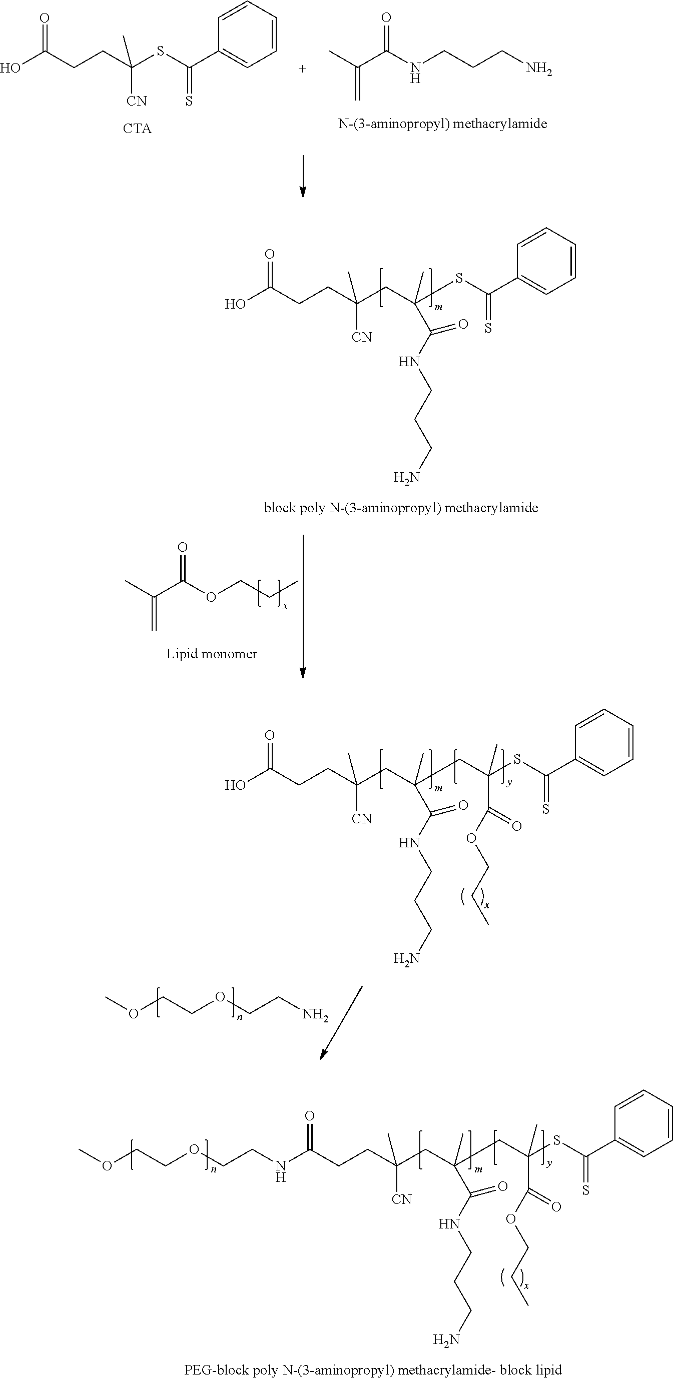

A chain transfer agent (4-cyanopentanoic acid dithiobenzoate (CTP)) was intermixed with an N-(3-aminopropyl)methacrylamide monomer to form a polymer of p(N-(3-aminopropyl)methacrylamide)-CTA. p(N-(3-aminopropyl)methacrylamide)-CTA was intermixed with a lipid monomer to form a copolymer (Block B) that includes a p(N-(3-aminopropyl)methacrylamide) block and a lipid block. The copolymer (Block B) was intermixed with an amine-terminated PEG to form a block copolymer that includes a PEG block (Block A), and a block that includes p(N-(3-aminopropyl)methacrylamide) and a lipid.

### Example 5

### Block Copolymer Synthesis

### MacroCTA (Subunit (A)) (pPEGMA or pHPMA)

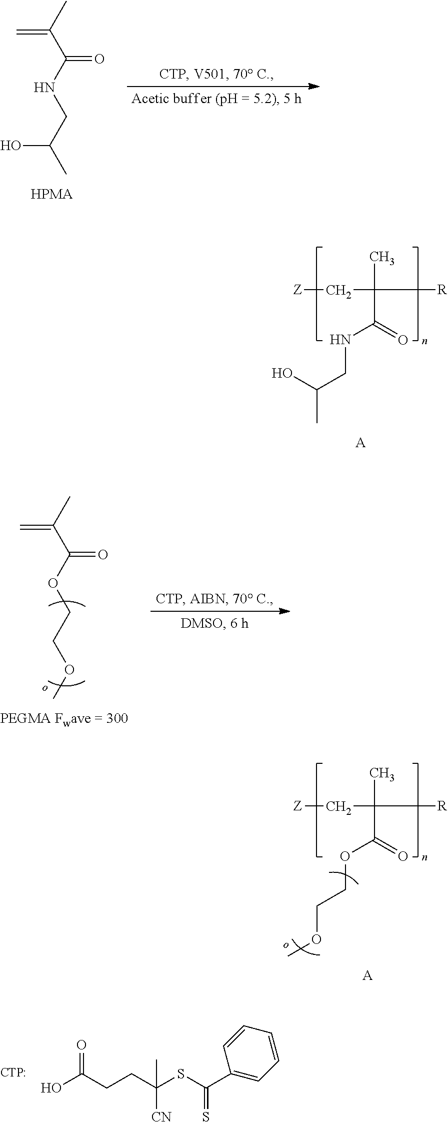

pHPMA and pPEGMA were synthesized by RAFT polymerization. pHPMA synthesis was conducted in an acetic buffer (pH=5.2, 0.27 M acetic acid and 0.73 M sodium acetate). CTP and 4,4′-azobis-4-cyanovaleric acid (V501) were used as the CTA and radical initiator, respectively. The initial CTA to monomer ratio, [CTA]ₒ/[M]ₒ, was 400/1. The initial CTA to initiator ratio, [CTA]ₒ/[I]ₒ, was 5/1. The final [M]ₒ was 1 M. After purging with Ar for 30 min, the reaction mixture was immersed into a preheated oil bath (70° C.) for 5 h. The polymer was isolated by dialysis (pH 3-4) using a 1000 MWCO membrane followed by lyophilization.

pPEGMA synthesis was conducted in DMSO (25 wt % monomer to solvent). CTP and AIBN were used as the CTA and radical initiator, respectively. The initial CTA to monomer ratio, [CTA]ₒ/[M]ₒ, was 100/1. The initial CTA to initiator ratio, [CTA]ₒ/[I]ₒ, was 5/1. After purging with Ar for 30 min, the reaction mixture was immersed into a preheated oil bath (70° C.) for 6 h. The polymer was isolated by dialysis against ultrapure water using 1000 MWCO membrane followed by lyophilization.

The resultant polymer can serve as a macro CTA to synthesize a block copolymer that can include a first subunit (A) and a second subunit (B) as illustrated in the below reaction scheme, wherein the first subunit (A) includes pHPMA or pPEGMA, respectively.

### Synthesis of Block Copolymer Including First Subunit (A) and Second Subunit (B)

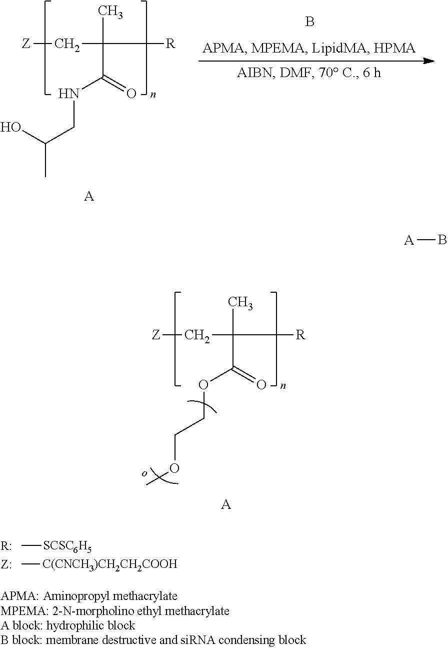

The B block chain extension was synthesized by RAFT. The polymer chain extension was conducted in DMSO (25 wt % total monomers to solvent) and used either pPEGMA₃₀₀ or pHPMA (“A”, as shown in the illustration above) as the macro CTA and AIBN as the radical initiator. The feeding ratio of each monomer can be varied, depending on the polymer design. The initial CTA to monomer ratio, [CTA]ₒ/[M]ₒ, was 300/1, while the initial CTA to initiator ratio, [CTA]ₒ/[I]ₒ, was 10/1. After purging with Ar for 30 min, the reaction was immersed into a preheated oil bath (70° C.) for 6 h. The polymer was isolated by dialysis, first against MeOH for 12 h and then against ultrapure water for an additional 24 h using a 1000 MWCO membrane, followed by lyophilization.

### Example 6

### Block Copolymer Synthesis

### Synthesis of Second Subunit (B)

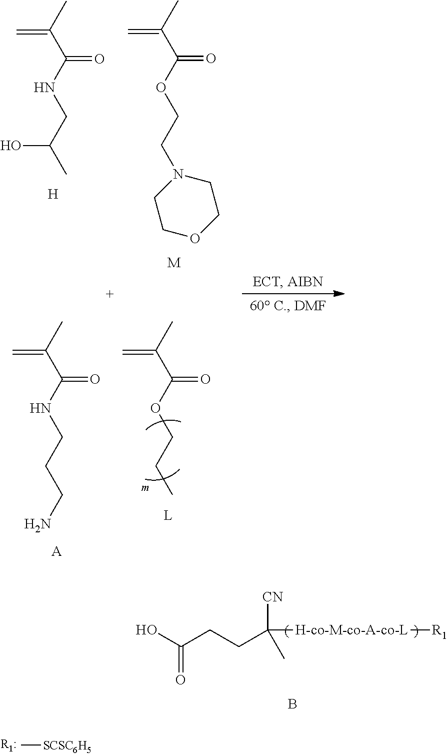

“co” in the above reaction scheme indicates a copolymer

The B subunit polymer was synthesized by RAFT polymerization. The B subunit polymer synthesis was conducted in DMF (25 wt % total monomers to solvent). Ethyl cyanovaleric trithiocarbonate (ECT) and AIBN were used as the CTA and the radical initiator, respectively. The feeding ratio of each monomer can be varied, depending on the block copolymer design. The initial CTA to monomer ratio, [CTA]ₒ/[M]ₒ, was 300/1. The initial CTA to initiator ratio, [CTA]ₒ/[I]ₒ, was 10/1. After purging with Ar for 30 min, the reaction was immersed into a preheated oil bath (70° C.) for 6 h. The B subunit polymer was isolated by dialysis against ultrapure water using a 1000 MWCO membrane followed by lyophilization. If the hydrophobic monomer cannot be removed by water dialysis, the polymer can be firstly precipitated into ether, redissolved in MeOH, and precipitated into ether (×2). The resultant polymer can subsequently be purified by dialysis following the procedure described above.

### Introduction of First Subunit (A) (PEG) by Postmodification Using Coupling Reaction

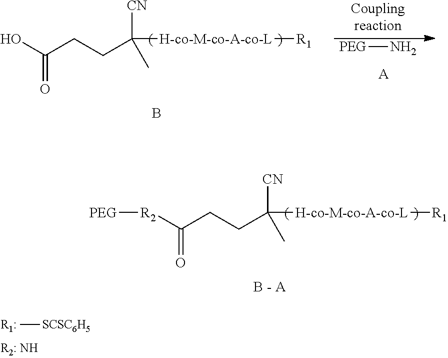

“co” in the above reaction scheme indicates a copolymer

PEG-NH₂ (M_{w}=2 kDa, 5 kDa, or 10 kDa) was added to the B subunit polymer by a DCC coupling reaction. The B subunit polymer (1 eq) and DCC (5 eq) were dissolved in MeOH. The solution was stirred for 30 min before addition of a PEG-NH₂ compound (1.1 eq) in MeOH. The reaction was continuously stirred under an inert environment overnight at room temperature. The polymer was purified by washing with acetone (×3) and was subsequently dialyzed against ultrapure water using a 1000 MWCO membrane for 48 h.

### Example 7

### Block Copolymer Synthesis

### Coupling Hydrophilic A Block with Endosomolytic Condensing B Block

A coupling reaction can be used to couple two individual polymers using carbodiimide chemistry. There are two ways to obtain the desired product. In some embodiments, a Boc-protected amine functional monomer can be used to avoid the unwanted products.

### Method A

### Using NH₂-CTP

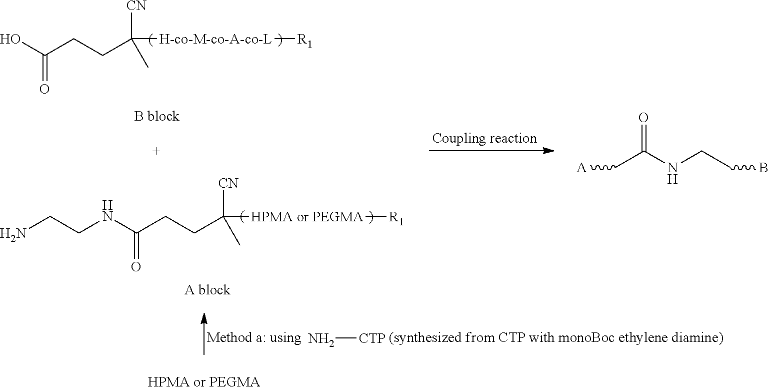

“co” in the above reaction scheme indicates a copolymer

Subunit A polymer (HPMA or PEGMA) was polymerized using an amine-functional CTP as the CTA, and subunit B polymer was polymerized by RAFT using CTP as the CTA, as described above. The amine-functional CTP was obtained by coupling mono-Boc ethylene diamine (e.g., N-Boc-ethylenediamine) to CTP, which was purified by flash column chromatography.

Subunit A polymer with a free amine was polymerized with subunit B polymer with a carboxylate via carbodiimide chemistry to form a block copolymer described herein. The resultant block copolymer was purified by dialysis against ultrapure water. The choice of MWCO membrane depends on the of the parent subunit polymers. For example, if the parent polymers are 15 kDa and 12 kDa each and the 15 kDa polymer is used as the excess reagent, the 20 kDa MWCO membrane is used in the dialysis process. The Boc was removed by treating with TFA. The block copolymer was further purified by dialysis or PD-10 to obtain the final, purified block copolymer.

### Method B

### Postmodification with Mono-Boc-Ethylene Diamine

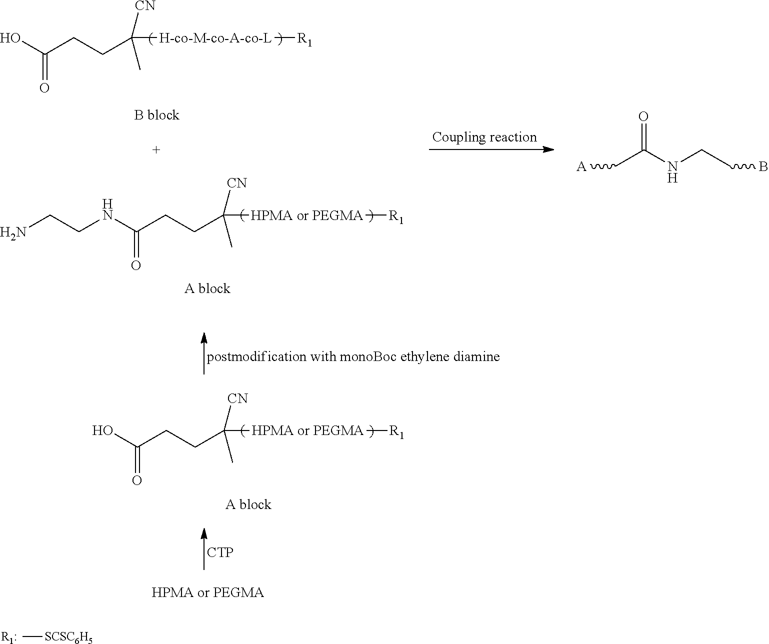

Both A and B subunit polymers were synthesized via RAFT polymerization using CTP as the CTA. The hydrophilic A subunit polymer was postmodified by a large excess of mono-Boc ethylene diamine. The Boc group was subsequently removed by TFA. TFA was removed by evaporator, and the subunit A polymer was purified using a PD-10 desalting column. The free amine of the subunit A polymer was coupled with the carboxylate of the subunit B polymer using carbodiimide chemistry, thus yielding the block copolymer. The block copolymer was purified by dialysis using the same procedure as described above.

### Example 8

### siRNA Transfection

The siRNA was a double stranded siRNA sequence with 21-mer targeting dscGFP. The sequences are as follows:

| (SEQ ID NO: 1) | |
|---|---|
| Sense: | 5′-GUGAUCUUCACCGACAAGATT |
| (SEQ ID NO: 2) | |
| Anti-sense: | 5′-UCUUGUCGGUGAAGAUCACTT |

Cells were seeded to 96-well plates at a density of 1×10⁴ cells per well one day before the transfection. siRNA (1.0 μg) was dissolved in distilled water and further diluted to 30 μL with OptiMEM (Invitrogen) to form a solution. Solutions of selected polymers were prepared by dissolving the selected polymers in dH₂O to a concentration of 5 mg/mL. The diluted siRNA solution and the polymer solutions were mixed and incubated at room temperature for 15 min. The mixture of the siRNA and the polymer (15 μL) were added to each well of the pre-seeded cells, mixed, and incubated at 37° C. incubator with 5% CO₂.

After about 48 hours, transfection was evaluated by measuring the expression of GFP under a fluorescence microscope. The absorbance of GFP was detected at 485-528 nm using a UV-vis microplate reader.

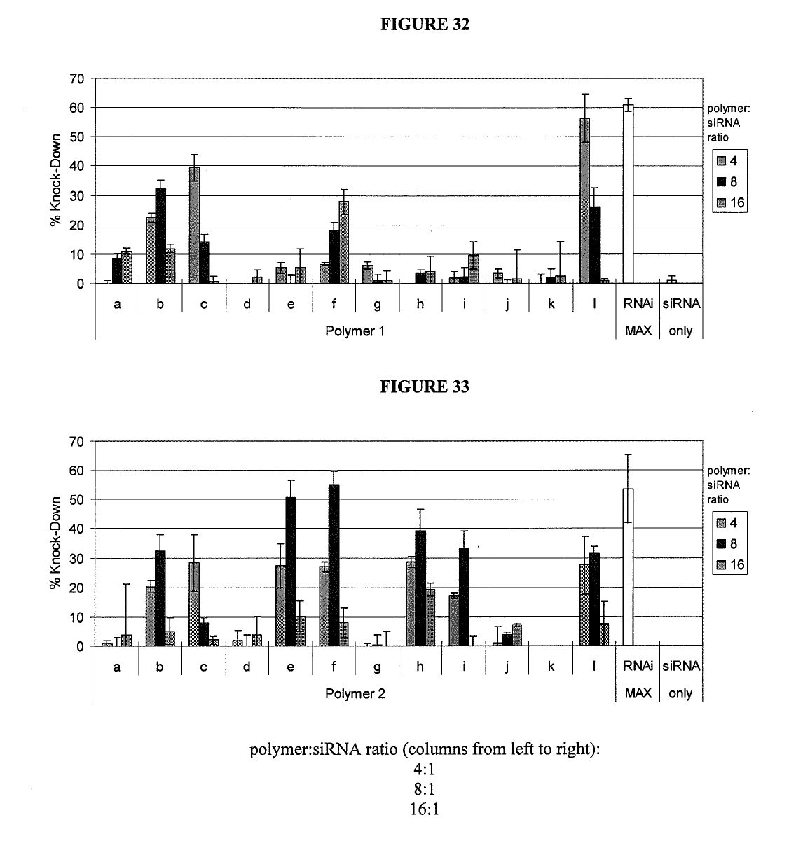

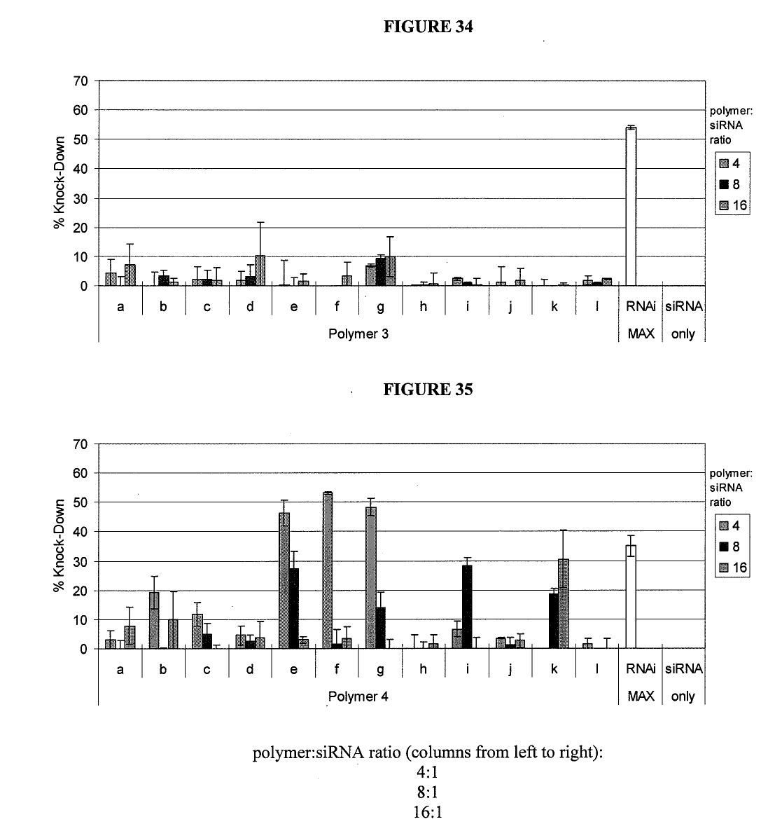

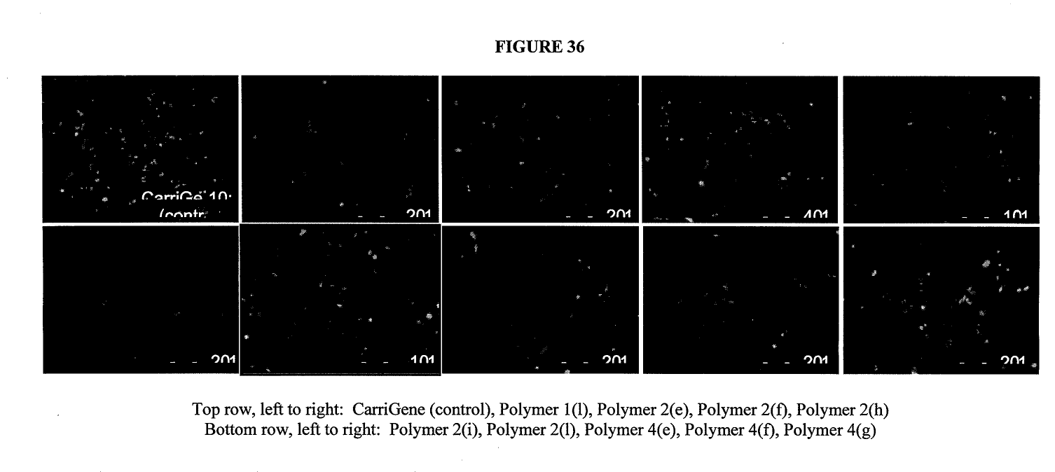

The results for transfection with polymers 1(a-1), 2(a-1), 3(a-1), and 4(a-1) are shown in FIGS. 32-35. For each polymer, transfection was evaluated at a ratio of polymer to siRNA (nitrogen to phosphate) of 4:1, 8:1, and 16:1. The results show that these polymers, in combination with siRNA, inhibit (or silence) green fluorescence protein expression as compared to the RNAi Max control.

### Example 9

### siRNA Transfection

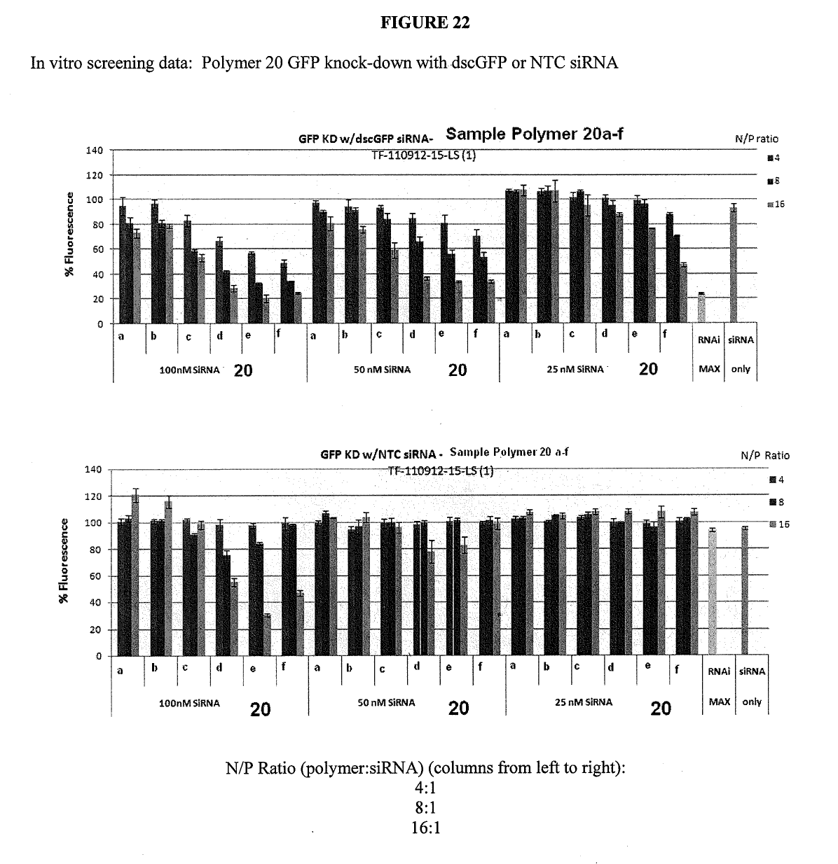

Solutions of Polymers 20(a-f) were intermixed with siRNA and transfected according to the procedure described in Example 8, except with respect to the concentrations of siRNA shown in FIG. 22. The results for transfection with polymers 20(a-f) are shown in FIG. 22. For each polymer, transfection was evaluated at a ratio of polymer to siRNA (nitrogen to phosphate) of 4:1, 8:1, and 16:1. The results show that these polymers, in combination with siRNA, inhibit (or silence) green fluorescence protein expression as compared to the RNAi Max control.

### Example 10

### Plasmid DNA (Gene) Transfection

### Cell Culture

CHO-K1 cells were maintained in F-12 medium containing 10% FBS, 100 units/mL penicillin and 100 μg/mL streptomycin at 37° C., 5% CO₂ and 100% humidity conditions. In this media, the cells were split every 3-4 days to avoid confluency.

### Plasmid DNA Preparation

peGFP-N1 plasmid DNA (GenBank Accession #U55762; SEQ. ID. NO.: 3) was purchased from BD Sciences Clontech company, which encodes a red-shifted variant of wild-type GFP that has been optimized for brighter fluorescence and higher expression in mammalian cells. The GFP protein is controlled by an immediate early promoter of CMV (P_{CMV IE}). The plasmids were amplified in DH5 E. coli and purified with Qiagen Plasmid Max Preparation Kit, and have an A260/A280 greater than 1.7.

### In Vitro Transfection

CHO-K1 cells were plated in 96-well tissue culture plates (1×10⁴ cells/well) and incubated overnight in F-12K medium with 10% FBS. For each well, an aliquot of 7.5 μL polymer solution in Opti-MEM at different concentrations were added to 7.5 μL of DNA solution containing 0.15 μg of pEGFP-N1 plasmid in Opti-MEM and mixed. The DNA and polymer mixture were incubated for 15 minutes at room temperature to allow for the formation of DNA-polymer complexes. The complexes were added to each well and incubated at 37° C., 5% CO₂ for 48 hours. The eGFP gene transfection efficiency was determined by GFP signal analysis. CarriGene (Kinovate Life Science) at a polymer to DNA ratio of 10:1 (weight ratio) was used as a positive control according to the protocol provided by manufacturer.

### Observation of GFP Signal

Green fluorescent signal in transfected cells was observed under a fluorescent microscope (Olympus, filter 520 nm). The cells were photographed using a 10× objective.

In order to quantify the transfection efficiency, the relative fluorescent unit of transfected cells was determined by fluorescent microplate reader (FLX 800, Bio-TEK Instruments Co Ltd).

Polymers 1(l), 2 (e, f, h, i), and 4 (e, f, g) at various polymer to DNA weight ratios were tested. The results are shown in FIG. 36. FIG. 36 indicates that the polymers exhibited a level of GFP expression, and accordingly, a level of transfection efficiency, that was comparable to the control polymer.

### Example 11

### Hemolysis Assay

### At pH 7.4

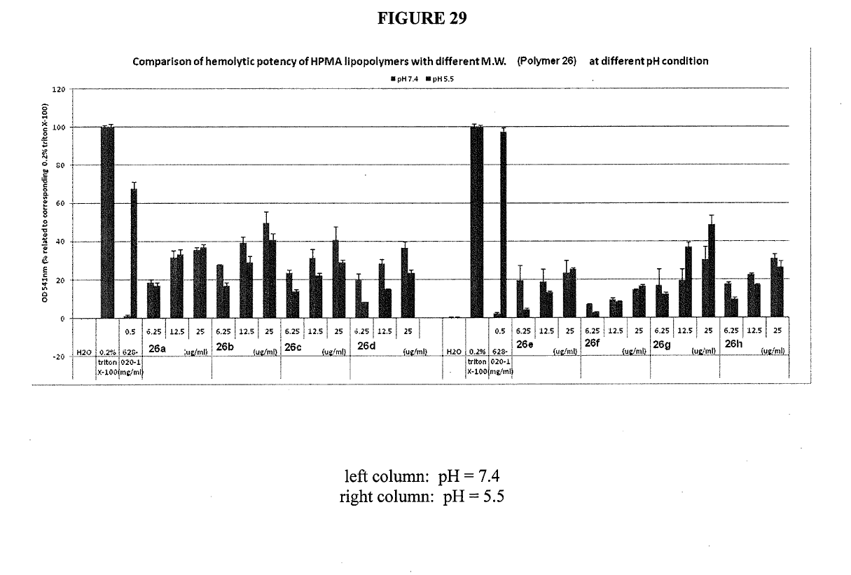

Hemolysis studies were carried out on polymers at various concentrations. Fresh blood samples of a rat were collected in an EDTA-Na rinsed tube. Red blood cells (RBC) were harvested by centrifugation at 3000 rpm for 10 min and rinsed with D-PBS (pH 7.4) 3 times. The red blood cell density was adjusted to 1×10⁸ per mL in D-PBS, and aliquoted cells (180 μL/well) were placed as a suspension in wells of a flat-bottom 96-well plate. The polymers (20 μL) were applied in various concentrations to corresponding wells in a triplicate format. D-PBS and 0.2% triton X-100 were used as the negative and positive controls, respectively. The plate was incubated at 37° C. for 1 hr on a 3-D rotating station. The samples were centrifuged at 850 g for 15 min. The supernatant (100 μL) was harvested from each well without disturbing the cell pellets, and transferred to a new flat-bottom 96-well plate. Optical density at 541 nm was measured. Data was presented as an average of % relative to the positive control, 0.2% triton X-100, for RBC hemolysis experiments. The results of the hemolysis assay at pH 7.4 are shown in FIGS. 29 (corresponding to selected variations of Polymer 26), 37 (corresponding to selected variations of Polymers 5, 6, and 7), and 38 (corresponding to selected variations of Polymers 1, 2, 3, and 4).

### At pH 5.5

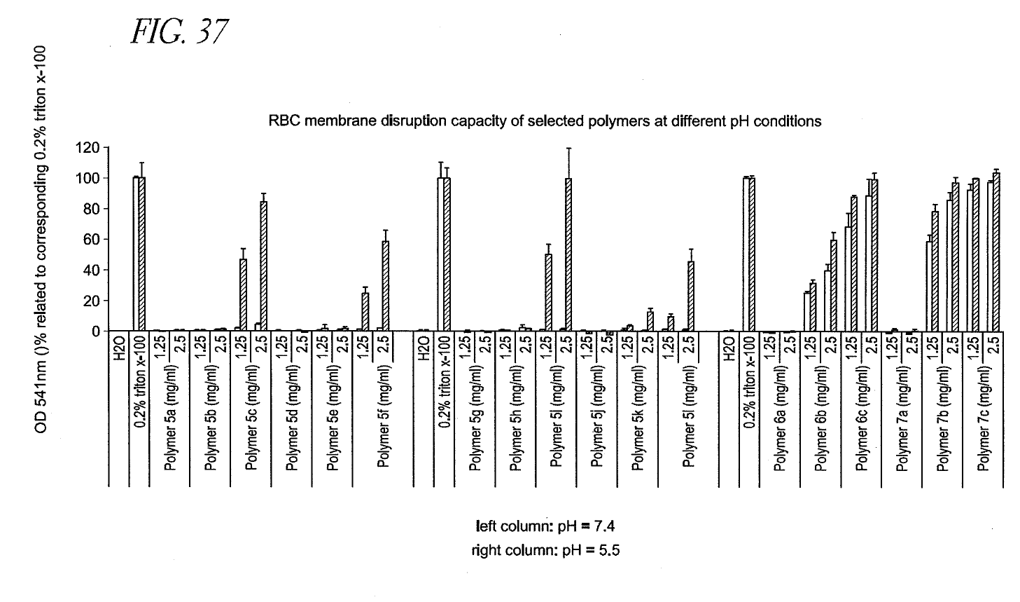

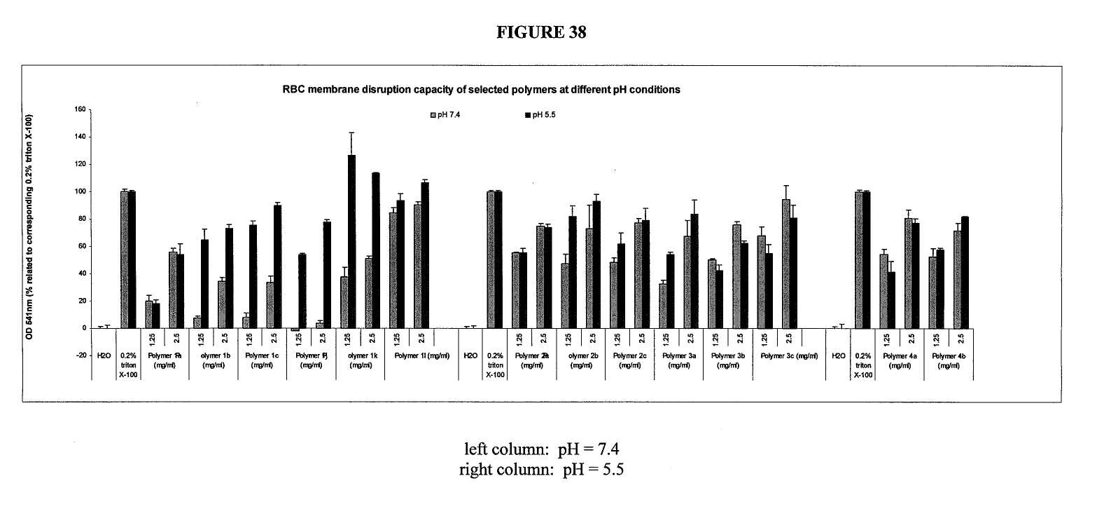

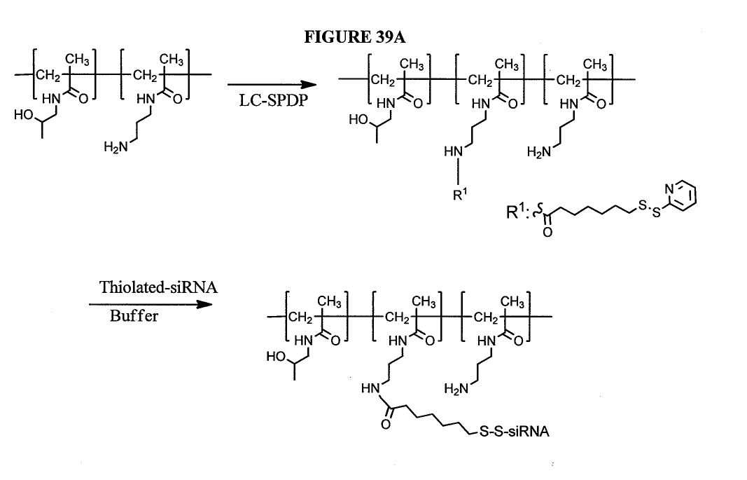

A protocol similar to the above was carried out, except that the buffer used was MES (2-(N-morpholinoethanesulfonic acid) (pH 5.5), not D-PBS. The results of the hemolysis assay at pH 5.5 are shown in FIGS. 29, 37, and 38.

As illustrated in FIGS. 29, 37, and 38, the red blood cell membrane disruption capacity of several polymers described herein was relatively low at pH 7.4 and relatively high at pH 5.5. This indicates that polymers described herein can become active in acidic environments and can operate as phase transition polymers.

### Example 12

### Cell Viability Assay

A solution of 3-(4,5-dimethylthiazol-2-yl)-2,5-diphenyltetrazolium bromide (MTT) is prepared by dissolving 250 mg of solid MTT in 50 mL of Dubecco PBS. The solution is stored at 4° C. After 48 hours of plasmid DNA transfection as described in Example 10, MTT solution (10 μL of the 5 mg/mL) is added to each well of the cells and incubated at 37° C. for 2-4 hours until purple crystal growth is observed. The solubilized solution (100 μL) is added and incubated at 37° C. overnight. The absorbance is detected at wavelength of 570 nm with the absorbance at 690 nm used as reference.

### Example 13

### siRNA Conjugation

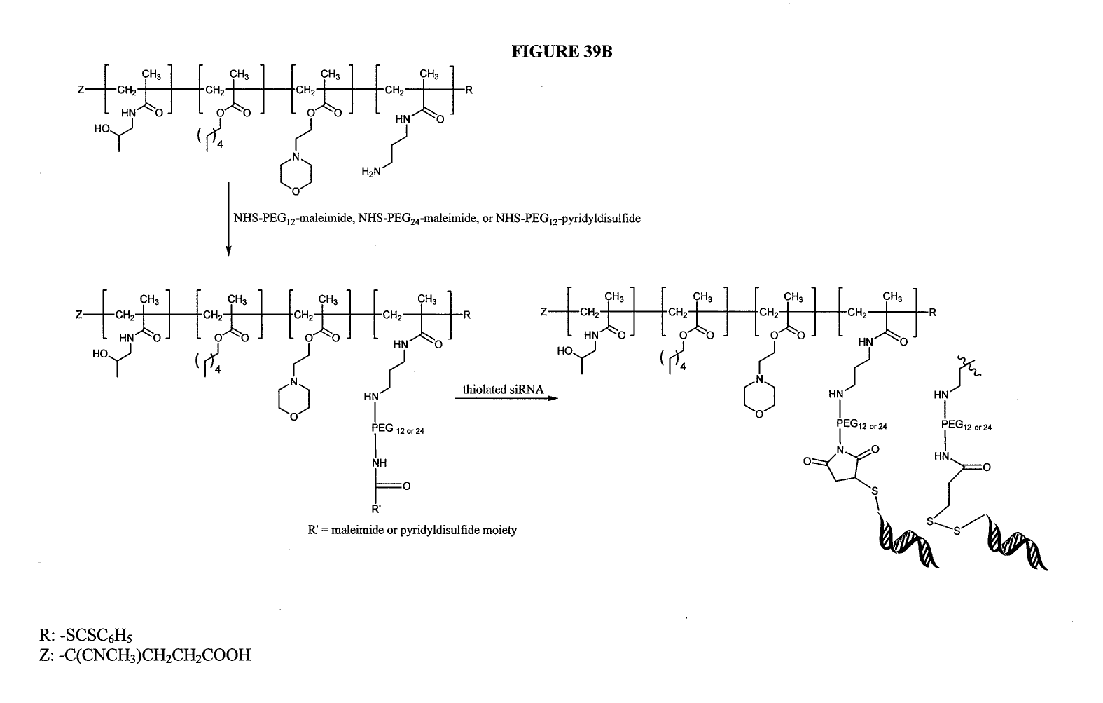

A polymer that includes a recurring unit of HPMA and a recurring unit of poly(N-(3-Aminopropyl methacrylamide) was intermixed with succinimidyl 6-(3-[2-pyridyldithio]-propionamido)hexanoate (LC-SPDP), a thiol-reactive agent, to yield a polymer covalently bonded to a thiol-reactive agent. The double-stranded siRNA described in SEQ ID NO:1 and SEQ ID NO:2 was modified to include a thiol group. The thiol-siRNA was incubated with the polymer to yield a polymer covalently bonded to siRNA, as illustrated in FIG. 39A. Additional recurring units can be added to the polymer according to the methods described herein.

Polymer 29, which included the recurring units referenced in Table 1 and had the structure illustrated in FIG. 39B, was synthesized according to methods described herein.

TABLE 1

| | | Hexyl | Morpholinoethyl | N-(3-Aminopropyl) |
|---|---|---|---|---|
| | HPMA | methacrylate | methacrylate | methacrylamide |
| Polymer | (mol %) | (mol %) | (mol %) | (mol %) |
| 29 | 25 | 40 | 30 | 5 |

Polymer 29 was intermixed with a thiol-reactive agent (NHS-PEG₁₂-maleimide, NHS-PEG₂₄-maleimide, or NHS-PEG₁₂-pyridyldisulfide) to covalently bond the thiol-reactive agent to the polymer. A stock solution of the polymer covalently bonded to the thiol-reactive agent was prepared in a buffer at a concentration of 10 mg/mL. A thiolated siRNA stock solution was prepared in RNAse-free water at a concentration of 3.75 mg/mL. An aliquot of polymer solution and an aliquot of buffer were transferred into a 1.5 mL eppendorf tube. An aliquot of the reduced thiolated siRNA buffer solution was added to the polymer solution. The details of each reaction condition are shown in Table 2, below.

TABLE 2

| | | Polymer/ | | | Total | |
|---|---|---|---|---|---|---|
| | Polymer | siRNA | siRNA | Buffer | volume | Linking |
| Polymer | (μL) | ratio | (μL) | (μL) | (μL) | group |
| 29a | 8.2 | 5 | 5.6 | 48.8 | 62.6 | PEG₁₂-MA |
| | | | | | | (maleimide) |
| 29b | 8.2 | 5 | 5.6 | 48.8 | 62.6 | PEG₂₄-MA |
| 29c | 8.2 | 5 | 5.6 | 48.8 | 62.6 | PEG₁₂-SPDP |
| | | | | | | (pyridine |
| | | | | | | disulfide) |

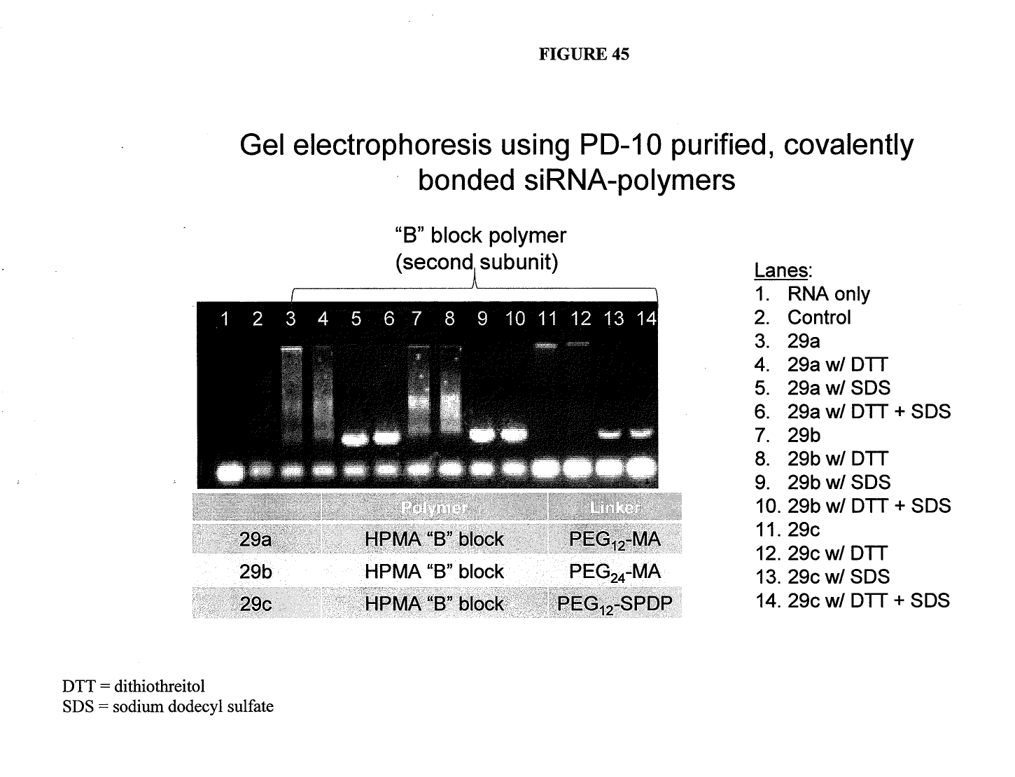

The reaction mixture was shaken, centrifuged and incubated at 37° C. overnight. The siRNA-polymer conjugate was confirmed by gel filtration and HPLC and further isolated by fractionation HPLC. The siRNA-polymer conjugate was purified using a PD-10 desalting column and was subject to a gel electrophoresis assay according to methods known to those skilled in the art. The results of the gel electrophoresis assay are illustrated in FIG. 45. As illustrated in FIG. 45, the bright band near the bottom of each lane is indicative of free siRNA (see, e.g., control lane 1). However, the longer bands (see, e.g., lanes 3-4 and 7-8) indicate slower-moving siRNA, and thus indicate an association between the siRNA and the polymer.

It will be understood by those of skill in the art that numerous and various modifications can be made without departing from the spirit of the present application. Therefore, it should be clearly understood that the forms of the present invention are illustrative only and not intended to limit the scope of the present invention.

What is claimed is:

1. A polymer comprising
a first recurring unit of Formula (I) having the structure: 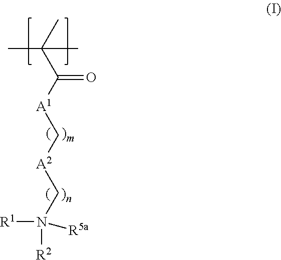
a second recurring unit of Formula (II) having the structure: 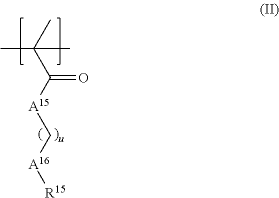
wherein
A¹ and A¹⁵ are independently selected from the group consisting of an oxygen atom and —N(R^{N}), wherein R^{N} is H or C₁-C₆ alkyl;
A² is a bond or —S—S—;
A¹⁶ is a bond, NH, or NHC(═O);
m is 1, 2, 3, 4, 5, or 6;
n and u are each independently 0, 1, 2, 3, 4, 5, or 6;
R¹ and R² are each independently selected from H and C₁-C₆ alkyl;
R^{5a} is absent or H, and if R^{5a} is H, the nitrogen atom to which R^{5a} is attached has an associated positive charge;
R¹⁵ is selected from the group consisting of C₄-C₂₄ alkyl, C₄-C₂₄ alkenyl, and an optionally substituted steryl.

2. The polymer of claim 1, wherein the second recurring unit has a structure selected from the group consisting of Formula (IIa) and (IIb):

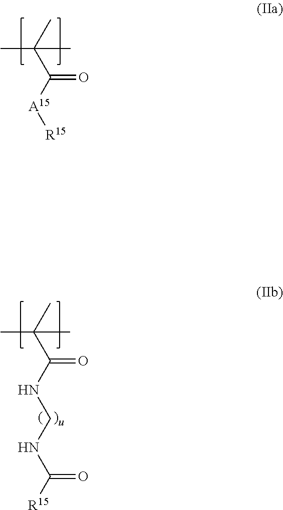

3. The polymer of claim 1, further comprising a third recurring unit of Formula (III) having the structure:

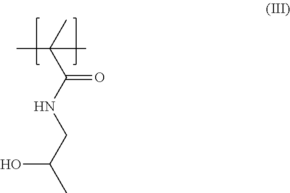

4. The polymer of claim 1, further comprising a recurring unit of Formula (A) having the following structure: 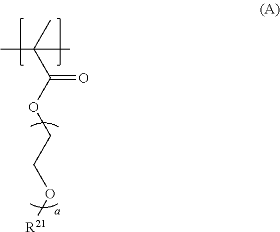
wherein a is an integer in the range of 1 to 100 and R²¹ is methyl or ethyl.

5. The polymer of claim 1, wherein A¹ is NH or an oxygen atom, and wherein A² is a bond or —S—S—.

6. The polymer of claim 1, wherein R¹ and R² are each H or CH₃.

7. The polymer of claim 1, wherein m is 2 or 3 and n is 0 or 2.

8. The polymer of claim 1, wherein R¹⁵ is selected from the group consisting of oleyl, lauryl, myristyl, palmityl, margaryl, stearyl, arachidyl, behenyl, lignoceryl and an optionally substituted steryl.

9. The polymer of claim 1, wherein R¹⁵ is C₄-C₉ alkyl.

10. The polymer of claim 1, further comprising a recurring unit of Formula (XIII) having the structure: 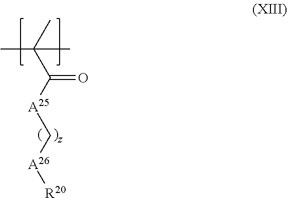
wherein
A²⁵ is selected from the group consisting of an oxygen atom and —N(R^{Q}), wherein R^{Q} is H or C₁-C₆ alkyl;
A²⁶ is a bond, NH, or NHC(═O);
z is 0, 1, 2, 3, 4, 5, or 6; and
R²⁰ comprises a group that has a pH transition point.

11. The polymer of claim 10, wherein the third recurring unit has a structure selected from the group consisting of Formula (XIIIa) and (XIIIb):

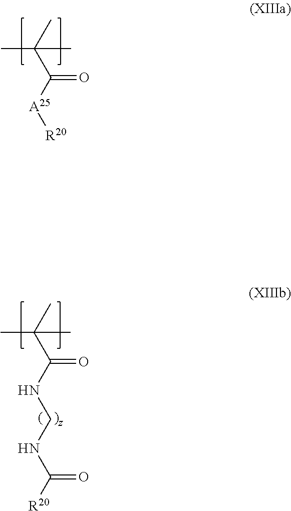

12. The polymer of claim 10, wherein A²⁵ is NH or an oxygen atom.

13. The polymer of claim 10, wherein A²⁶ is a bond or NH, and z is 0 or 3.

14. The polymer of claim 10, wherein R²⁰ is hydrophilic at a pH≧the transition point.

15. The polymer of claim 10, wherein R²⁰ is hydrophobic at a pH<the transition point.

16. The polymer of claim 10, wherein R²⁰ is a group that comprises. an imidazolyl group, a 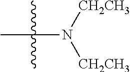
group, a 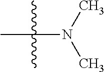
group or a morpholinyl group.

17. The polymer of claim 10, wherein R²⁰ has the following structure: —C(═O)—(CH₂)₁₋₄—C(═O)OR^{B} at a pH≧the transition point, wherein R^{B} is an alkali metal.

18. The polymer of claim 10, wherein R²⁰ has the following structure: 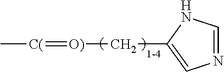
at a pH≧the transition point.

19. The polymer of claim 10, wherein R²⁰ has the following structure: 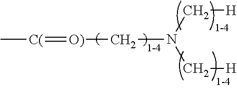
at a pH≧the transition point.

20. The polymer of claim 10, wherein R²⁰ has the following structure: 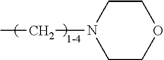
at a pH≧the transition point.

21. The polymer of claim 10, wherein R²⁰ has the following structure: 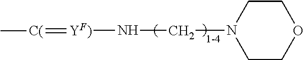
at a pH≧the transition point, wherein Y^{F} is S or O.

22. The polymer of claim 10, wherein the transition point is at a pH<7.4.

23. The polymer of claim 10, wherein the transition point is at a pH<5.

24. The polymer of claim 1, further comprising a nucleic acid associated with the polymer, wherein the nucleic acid is selected from the group consisting of DNA, RNA siRNA and antisense.

25. The polymer of claim 10, wherein an amount of the polymer is more insoluble in a solvent at a pH less than the transition point compared to the same amount of the same polymer in the same solvent at a pH greater than or equal to the transition point.

26. A block copolymer, comprising:
a first subunit comprising a recurring unit selected from the group consisting of Formula (E) and Formula (F): 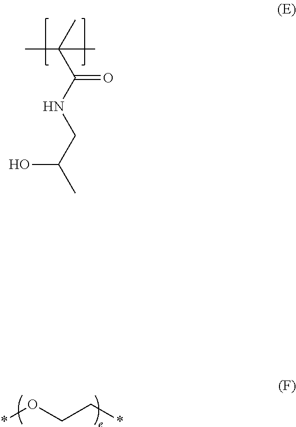
wherein e in an integer in the range of from about 1 to about 15; and
a second subunit comprising the polymer of claim 1.

27. A pharmaceutical composition comprising a pharmaceutically acceptable carrier and the polymer of claim 1.

28. A method for transfecting a cell comprising delivering to the cell at least one of the polymer of claim 24.

29. A method for treating a tumor comprising administering an effective amount of the polymer of claim 24 to a subject with the tumor.

30. A method for treating a tumor comprising contacting a tumor cell with an effective amount of the polymer of claim 24 to a subject with the tumor.


SEQUENCE LISTING (3 sequences)

SEQ ID NO: 1 - DNA, 21 bp, Artificial Sequence
  Synthetic sense siRNA
gugaucuucaccgacaagatt

SEQ ID NO: 2 - DNA, 21 bp, Artificial Sequence
  Synthetic antisense siRNA
ucuugucggugaagaucactt

SEQ ID NO: 3 - DNA, 4733 bp, Artificial Sequence
  pEGFP-N1
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgctaccggactcagatctcgagctcaagcttcgaattctgcagtcgacggtaccgcgggcccgggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaaggcgtaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtcctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaagatcgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccctagggggaggctaactgaaacacggaaggagacaataccggaaggaacccgcgctatgacggcaataaaaagacagaataaaacgcacggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcctcaggttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgccatgcat